(19) United States
(12) Patent Application Publication
(10) Pub. No.: US 2026/0315805 A1
(43) Pub. Date: Oct. 15, 2026

(54) NERVE STIMULATION FOR PAIN CONTROL

(71) Applicant: Amber Therapeutics Holdings Limited, London (GB)

(72) Inventors: Timothy Denison, London (GB); Stefan De Wachter, London (GB); Charles Knowles, London (GB); Aidan Crawley, London (GB)

(21) Appl. No.: 19/111,108

(22) PCT Filed: Sep. 14, 2023
(86) PCT No.: PCT/GB2023/052379

§ 371 Date: Mar. 12, 2025

Related U.S. Application Data
(60) Provisional application No. 63/375,834, filed on Sep. 15, 2022.

(51) Int. Cl. A61N 1/36

(52) U.S. Cl. CPC A61N 1/36139; A61N 1/36071

(57) ABSTRACT

Provided are devices and methods for reducing pain of an individual in need thereof. The devices comprise an implantable sensor and a stimulator electrode that is implanted into the pelvic area of the individual. The implanted sensor can detect a signal associated with an episode of pain from the individual, and the stimulator electrode can provide an electrical stimulation adapted to the signal detected in order to reduce pain associated with the episode.

### CROSS-REFERENCE

This application claims the benefit of U.S. Provisional Application No. 63/375,834 filed Sep. 15, 2022, which is incorporated by reference in its entirety.

### BACKGROUND

Pelvic pain, or pain in the pelvic area, has many causes but commonly involves contraction of pelvic floor muscles and activity of nerves that innervate these muscles. Chronic pelvic pain (CPP) is non-malignant pain perceived in the pelvic area in either men or women that lasts for 6 months or longer. CPP is a complex, multifactorial condition that is very disabling for patients. CPP may come and go or be constant with episodes of exacerbating pain.

### SUMMARY

Chronic pelvic pain (CPP) can impact quality of life, often causing episodes of severe discomfort and exacerbating pain. Common symptoms of CPP include but are not limited to neuropathic symptoms like paresthesia, numbness, burning, lancinating pain, in the pelvic, anus and/or genitals. Episodes of pain associated with CPP may frequently occur with sitting, urinating, defecating, attempts at urinating, attempts at defecating, or sexual intercourse and may be exacerbated with these activities. Approaches to treat CPP by electrically stimulating a large section of the affected area (e.g, pelvic area) or transcutaneously may have limited success at alleviating the symptoms of pain. As such, targeting specific nerves for electrical stimulation that is adapted to the individual's pain response may provide a highly efficacious treatment for CPP and other pain symptoms.

Aspects of the disclosure herein provide methods for reducing pain of an individual, the method comprising: (a) implanting a sensor and a stimulator electrode within a pelvic area of the individual; (b) sensing with the implanted sensor a parameter associated with an episode of pain of the individual; and (b) providing an adapted electrical stimulation with the implanted stimulator electrode that reduces pain of the individual, wherein at least one of an intensity, a frequency, or a duration of the adapted electrical stimulation varies according to the parameter detected in step (b). In some embodiments, the stimulator electrode comprises a first stimulator and wherein the first stimulator is implanted at or adjacent to a first anatomical site. In some embodiments, the stimulator electrode further comprises a second stimulator and wherein the second stimulator is implanted at or adjacent to a second anatomical site. In some embodiments, the method further comprises providing a base electrical stimulation with the implanted stimulator electrode before the adapted electrical stimulation is provided. In some embodiments, the adapted electrical stimulation is different from the base electrical stimulation. In some embodiments, at least one of the intensity or the frequency of the adapted electrical stimulation is different from that of the base electrical stimulation. In some embodiments, the adapted electrical stimulation boosts the base electrical stimulation. In some embodiments, the adapted electrical stimulation, alone or together with the base electrical stimulation, reduces the pain of the episode. In some embodiments, the adapted electrical stimulation comprises a first stimulation pattern and a second stimulation pattern, wherein the first stimulation pattern is provided by the first stimulator and the second stimulation pattern is provided by the second stimulator. In some embodiments, the first and second stimulation patterns comprise excitation values for intensity, frequency, phase, pulse width, or any combination thereof. In some embodiments, the first and second stimulation patterns are the same. In some embodiments, the first and second stimulation patterns differ in intensity, frequency, phase, pulse width, or any combination thereof. In some embodiments, the method further comprises using software to generate the first and second stimulation patterns based on at least the parameter. In some embodiments, the generated first and second stimulation patterns are configured to reduce pain of the episode based on at least the parameter. In some embodiments, the parameter comprises a first parameter and a second parameter and wherein the adapted electrical stimulation is further sustained, modified, redirected, withheld, or cancelled based on the second parameter and wherein the second parameter is later in time to the first parameter. In some embodiments, the sensor and the stimulator electrode are electrically coupled to a processor. In some embodiments, the parameter is provided by the individual via a controller in wireless communication with the processor. In some embodiments, the sensor is configured to transmit data to the processor, and wherein the data is associated with the parameter that is detected by the sensor. In some embodiments, the parameter associated with the episode comprises an electrical parameter, a pressure parameter, a motion parameter, or any combination thereof. In some embodiments, the parameter associated with the episode comprises a physiological signal, EMG signal, ENG signal, digital signal, patient actuated signal based on perception of pain or increased pain, pressure signal, muscle contraction event signal, motion signal, orientation signal, posture signal, GPS signal, speed signal, location signal, time of day signal, time interval signal, or any combination thereof. In some embodiments, the sensor is configured to detect the parameter. In some embodiments, the sensor comprises a sensor electrode, receiver, pressure sensor, accelerometer, gyroscope, magnetometer, global positioning system, or any combination thereof. In some embodiments, the parameter detected by the receiver, pressure sensor, accelerometer, gyroscope, magnetometer, global positioning system, or any combination thereof is associated with a pressure, muscle contraction event, motion, orientation, posture, GPS reading, speed, location, time of day, time interval, or any combination thereof. In some embodiments, the parameter detected by the sensor electrode, receiver, pressure sensor, or any combination thereof is associated with a physiological event, EMG signal, ENG signal, digital signal, patient actuated input based on perception of pain or increased pain, pressure, muscle contraction event, or any combination thereof. In some embodiments, the receiver comprises a controller receiver, digital receiver, radio receiver, or any combination thereof. In some embodiments, the sensor is configured to detect activity of a muscle of the individual, and wherein the activity is associated with an EMG signal, ENG signal, digital signal, patient actuated signal based on perception of pain or increased pain, pressure signal, muscle contraction event signal, or any combination thereof. In some embodiments, the muscle comprises a pelvic floor muscle. In some embodiments, the parameter is associated with contraction, increased tone, or any combination thereof of the pelvic floor muscle. In some embodiments, the sensor is configured to detect activity of a sensed nerve of the individual, and wherein the activity is associated with an ENG signal of the sensed nerve. In some embodiments, the sensed nerve comprises a motor nerve fiber, a larger diameter afferent nerve, or any combination thereof. In some embodiments, the sensed nerve comprises a pudendal nerve, a sacral nerve, a nerve of the pelvic plexus, or any combination thereof. In some embodiments, the episode of pain comprises a recurring pain, an exacerbating pain, or any combination thereof. In some embodiments, the pain comprises pelvic pain. In some embodiments, the pain further comprises chronic pelvic pain. In some embodiments, the pain is associated with interstitial cystitis, painful bladder syndrome, chronic prostatitis, urethral pain syndrome, vagina pain, vulvodynia, vestibulodynia, radiation vaginitis, penile pain syndrome, proctalgia fugax, anorectal pain (e.g., unspecified functional anorectal pain syndrome), phantom rectum syndrome, levator ani syndrome, pelvic floor muscle pain syndrome, pelvic floor muscle pain dysfunction, pelvic floor myalgia, urethral pain syndrome, coccygodynia, pudendal neuralgia, or any combination thereof. In some embodiments, the episode of pain of the individual occurs when the individual sits, changes posture, urinates, defecates, attempts to urinate, attempts to defecate, has sexual intercourse, or any combination thereof. In some embodiments, the parameter is associated with the individual sitting, changing posture, urinating, defecating, attempting to urinate, attempting to defecate, having sexual intercourse, or any combination thereof. In some embodiments, the pain is associated with clinical or sub-clinical inflammation. In some embodiments, the pain is associated with compression of at least one nerve ending during a striated muscle spasm. In some embodiments, the pain is associated with compression of at least one nerve ending during an anatomical compression event. In some embodiments, the pain comprises neuropathic pain. In some embodiments, the pain is associated with nerve damage or injury. In some embodiments, the pain is associated with dysregulated visceral smooth muscle activity. In some embodiments, the pain of the individual has diurnal variation. In some embodiments, the parameter comprises temporal or circadian functions. In some embodiments, the first anatomical site, the second anatomical site, or any combination thereof is adjacent to or at a nerve. In some embodiments, the nerve comprises a motor nerve fiber, a larger diameter afferent nerve, or any combination thereof. In some embodiments, the nerve gates peripheral nociception, spinal activity, or any combination thereof. In some embodiments, the nerve stimulates muscle contraction, modulates muscle activity, or any combination thereof. In some embodiments, the nerve comprises a pudendal nerve, a sacral nerve, a nerve of the pelvic plexus, or any combination thereof. In some embodiments, a pudendal nerve, a sacral nerve, or a combination thereof is electrically stimulated by the stimulator electrode. In some embodiments, stimulation is provided by the first stimulator to a first nerve before the parameter is detected and, after the parameter is detected, stimulation is provided to the first nerve, a second nerve, or any combination thereof. In some embodiments, the first anatomical site, the second anatomical site, or any combination thereof is adjacent to or at a muscle. In some embodiments, the first anatomical site, the second anatomical site, or any combination thereof is adjacent to or at a pelvic muscle. In some embodiments, the first anatomical site is adjacent to or at a first pudendal nerve, and wherein the second anatomical site is adjacent to or at a sacral nerve, the first pudendal nerve, or a second pudendal nerve.

Provided herein are methods of reducing pain of an individual using a processor, the method comprising: (a) causing, by the processor, an implanted stimulator electrode to deliver a base electrical stimulation in a pelvic area of the individual; (b) receiving, by the processor, a parameter associated with an episode of pain of the individual from an implanted sensor, wherein the implanted sensor is configured to detect the parameter; (c) analyzing, by the processor, the parameter to determine at least one of an intensity, a frequency, or a duration of an adapted electrical stimulation; and (d) causing, by the processor, the implanted stimulator electrode to continue, alter, or terminate the base electrical stimulation and provide the adaptive electrical stimulation, wherein the adapted electrical stimulation, alone or together with the base electrical stimulation, reduces pain of the individual. In some embodiments, the parameter is indicative of exacerbating pain. In some embodiments, the parameter is indicative of chronic pain. In some embodiments, the parameter comprises a first value and a second value, and wherein the second value is used to determine cessation of the adapted electrical stimulation, and wherein causing, by the processor, the implanted electrode to cease providing the adapted electrical stimulation and revert to delivering only the base electrical stimulation, and wherein the second value is later in time to the first value. In some embodiments, the stimulator electrode comprises a first stimulator and wherein the first stimulator is implanted at or adjacent to a first anatomical site. In some embodiments, the stimulator electrode further comprises a second stimulator and wherein the second stimulator is implanted at or adjacent to a second anatomical site. In some embodiments, the method further comprises providing a base electrical stimulation with the implanted stimulator electrode before the adapted electrical stimulation is provided. In some embodiments, the adapted electrical stimulation is different from the base electrical stimulation. In some embodiments, at least one of the intensity or the frequency of the adapted electrical stimulation is different from that of the base electrical stimulation. In some embodiments, the adapted electrical stimulation boosts the base electrical stimulation. In some embodiments, the adapted electrical stimulation, alone or together with the base electrical stimulation, reduces the pain of the episode. In some embodiments, the adapted electrical stimulation comprises a first stimulation pattern and a second stimulation pattern, wherein the first stimulation pattern is provided by the first stimulator and the second stimulation pattern is provided by the second stimulator. In some embodiments, the first and second stimulation patterns comprise excitation values for intensity, frequency, phase, pulse width, or any combination thereof. In some embodiments, the first and second stimulation patterns are the same. In some embodiments, the first and second stimulation patterns differ in intensity, frequency, phase, pulse width, or any combination thereof. In some embodiments, the method further comprises using software to generate the first and second stimulation patterns based on at least the parameter. In some embodiments, the generated first and second stimulation patterns are configured to reduce pain of the episode based on at least the parameter. In some embodiments, the parameter comprises a first parameter and a second parameter and wherein the adapted electrical stimulation is further sustained, modified, redirected, withheld, or cancelled based on the second parameter and wherein the second parameter is later in time to the first parameter. In some embodiments, the sensor and the stimulator electrode are electrically coupled to a processor. In some embodiments, the parameter is provided by the individual via a controller in wireless communication with the processor. In some embodiments, the sensor is configured to transmit data to the processor, and wherein the data is associated with the parameter that is detected by the sensor. In some embodiments, the parameter associated with the episode comprises an electrical parameter, a pressure parameter, a motion parameter, or any combination thereof. In some embodiments, the parameter associated with the episode comprises a physiological signal, EMG signal, ENG signal, digital signal, patient actuated signal based on perception of pain or increased pain, pressure signal, muscle contraction event signal, motion signal, orientation signal, posture signal, GPS signal, speed signal, location signal, time of day signal, time interval signal, or any combination thereof. In some embodiments, the sensor is configured to detect the parameter. In some embodiments, the sensor comprises a sensor electrode, receiver, pressure sensor, accelerometer, gyroscope, magnetometer, global positioning system, or any combination thereof. In some embodiments, the parameter detected by the receiver, pressure sensor, accelerometer, gyroscope, magnetometer, global positioning system, or any combination thereof is associated with a pressure, muscle contraction event, motion, orientation, posture, GPS reading, speed, location, time of day, time interval, or any combination thereof. In some embodiments, the parameter detected by the sensor electrode, receiver, pressure sensor, or any combination thereof is associated with a physiological event, EMG signal, ENG signal, digital signal, patient actuated input based on perception of pain or increased pain, pressure, muscle contraction event, or any combination thereof. In some embodiments, the receiver comprises a controller receiver, digital receiver, radio receiver, or any combination thereof. In some embodiments, the sensor is configured to detect activity of a muscle of the individual, and wherein the activity is associated with an EMG signal, ENG signal, digital signal, patient actuated signal based on perception of pain or increased pain, pressure signal, muscle contraction event signal, or any combination thereof. In some embodiments, the muscle comprises a pelvic floor muscle. In some embodiments, the parameter is associated with contraction, increased tone, or any combination thereof of the pelvic floor muscle. In some embodiments, the sensor is configured to detect activity of a sensed nerve of the individual, and wherein the activity is associated with an ENG signal of the sensed nerve. In some embodiments, the sensed nerve comprises a motor nerve fiber, a larger diameter afferent nerve, or any combination thereof. In some embodiments, the sensed nerve comprises a pudendal nerve, a sacral nerve, a nerve of the pelvic plexus, or any combination thereof. In some embodiments, the episode of pain comprises a recurring pain, an exacerbating pain, or any combination thereof. In some embodiments, the pain comprises pelvic pain. In some embodiments, the pain further comprises chronic pelvic pain. In some embodiments, the pain is associated with interstitial cystitis, painful bladder syndrome, chronic prostatitis, urethral pain syndrome, vagina pain, vulvodynia, vestibulodynia, radiation vaginitis, penile pain syndrome, proctalgia fugax, anorectal pain (e.g., unspecified functional anorectal pain syndrome), phantom rectum syndrome, levator ani syndrome, pelvic floor muscle pain syndrome, pelvic floor muscle pain dysfunction, pelvic floor myalgia, urethral pain syndrome, coccygodynia, pudendal neuralgia, or any combination thereof. In some embodiments, the episode of pain of the individual occurs when the individual sits, changes posture, urinates, defecates, attempts to urinate, attempts to defecate, has sexual intercourse, or any combination thereof. In some embodiments, the parameter is associated with the individual sitting, changing posture, urinating, defecating, attempting to urinate, attempting to defecate, having sexual intercourse, or any combination thereof. In some embodiments, the pain is associated with clinical or sub-clinical inflammation. In some embodiments, the pain is associated with compression of at least one nerve ending during a striated muscle spasm. In some embodiments, the pain is associated with compression of at least one nerve ending during an anatomical compression event. In some embodiments, the pain comprises neuropathic pain. In some embodiments, the pain is associated with nerve damage or injury. In some embodiments, the pain is associated with dysregulated visceral smooth muscle activity. In some embodiments, the pain of the individual has diurnal variation. In some embodiments, the parameter comprises temporal or circadian functions. In some embodiments, the first anatomical site, the second anatomical site, or any combination thereof is adjacent to or at a nerve. In some embodiments, the nerve comprises a motor nerve fiber, a larger diameter afferent nerve, or any combination thereof. In some embodiments, the nerve gates peripheral nociception, spinal activity, or any combination thereof. In some embodiments, the nerve stimulates muscle contraction, modulates muscle activity, or any combination thereof. In some embodiments, the nerve comprises a pudendal nerve, a sacral nerve, a nerve of the pelvic plexus, or any combination thereof. In some embodiments, a pudendal nerve, a sacral nerve, a pelvic plexus nerve, or a combination thereof is electrically stimulated by the stimulator electrode. In some embodiments, stimulation is provided by the first stimulator to a first nerve before the parameter is detected and, after the parameter is detected, stimulation is provided to the first nerve, a second nerve, or any combination thereof. In some embodiments, the first anatomical site, the second anatomical site, or any combination thereof is adjacent to or at a muscle. In some embodiments, the first anatomical site, the second anatomical site, or any combination thereof is adjacent to or at a pelvic muscle. In some embodiments, the first anatomical site is adjacent to or at a first pudendal nerve, and wherein the second anatomical site is adjacent to or at a sacral nerve, the first pudendal nerve, or a second pudendal nerve. In some embodiments, a first anatomical site is adjacent to or at a first pudendal nerve, a first sacral nerve, or a first pelvic plexus nerve. In some embodiments, a second anatomical site is adjacent to or at a second pudendal nerve, a second sacral nerve, or a second pelvic plexus nerve.

Described herein are methods of data processing, the method comprising: (a) receiving a parameter associated with an episode of pain of the individual from an implanted senso in a pelvic region of the individual, the implanted sensor being configured to detect the parameter; (b) analyzing the parameter, comprising classifying the parameter against a comparative parameter of a dataset; and (c) generating a stimulation pattern configured to reduce pain of the episode when the stimulation pattern is performed by an implanted electrode in the pelvic region of the individual, wherein at least one of an intensity, a frequency, or a duration of the stimulation pattern varies according to analysis of the parameter. In some embodiments, the sensor, the implanted electrode, or a combination thereof, are implanted at or adjacent to a pudendal nerve, sacral nerve, pelvic plexus nerve, or any combination thereof. In some embodiments, the parameter comprises a bio-signal, extrinsic signal, learned signal, or any combination thereof. In some embodiments, the dataset comprises data generated by a user, a subject, a population, or any combination thereof. In some embodiments, the comparative parameter comprises an innate value, extrinsic value, learned value, or any combination thereof. In some embodiments, the method further comprises using software configured to generate the first and second stimulation patterns based on the parameter. In some embodiments, the software comprises a machine learning model, and wherein the machine learning model is configured to classify the parameter received by the implanted sensor and generate the stimulation patterns. In some embodiments, the machine learning model comprises training a classifier of user-specific activity based on at least one of a GPS reading, time of day, or motion. In some embodiments, the comparative parameter comprises a bio-signal, extrinsic signal, learned signal, or any combination thereof. In some embodiments, the implanted electrode comprises a stimulator electrode. In some embodiments, the stimulator electrode comprises a first stimulator and wherein the first stimulator is implanted at or adjacent to a first anatomical site. In some embodiments, the stimulator electrode further comprises a second stimulator, and wherein the second stimulator is implanted at or adjacent to a second anatomical site. In some embodiments, the first anatomical site is adjacent to or at a first pudendal nerve, a first sacral nerve, or a first pelvic plexus nerve. In some embodiments, the second anatomical site is adjacent to or at a second pudendal nerve, a second sacral nerve, or a second pelvic plexus nerve. In some embodiments, the stimulation pattern comprises a first stimulation pattern and a second stimulation pattern, wherein the first stimulation pattern is provided by the first stimulator, and the second stimulation pattern is provided by the second stimulator. In some embodiments, the first and second stimulation patterns comprise excitation values for intensity, frequency, phase, pulse width, or any combination thereof. In some embodiments, the first and second stimulation patterns are the same. In some embodiments, the first and second stimulation patterns differ in intensity, frequency, phase, pulse width, or any combination thereof. In some embodiments, the method further comprises providing a base electrical stimulation before providing the stimulation pattern. In some embodiments, the base electrical stimulation is different from the stimulation pattern. In some embodiments, at least one of the intensity or frequency of the stimulation pattern is different from at least one of the intensity or frequency of the base electrical stimulation. In some embodiments, the stimulation pattern boosts the base electrical stimulation. In some embodiments, the stimulation pattern, alone or together with the base electrical stimulation, reduces the pain of the individual. In some embodiments, the stimulation pattern is generated by software. In some embodiments, the parameter comprises a first parameter and a second parameter and wherein the stimulation pattern is further sustained, modified, redirected, withheld, or cancelled based on the second parameter and wherein the second parameter is later in time to the first parameter. In some embodiments, the implanted sensor and implanted electrode are electrically coupled to a processor. In some embodiments, the parameter is provided by the individual via a controller in wireless communication with the processor. In some embodiments, the sensor is configured to transmit data to the processor, and wherein the data is associated with the parameter that is detected by the sensor. In some embodiments, the parameter associated with the episode of pain of the individual comprises an electrical parameter, a pressure parameter, a motion parameter, or any combination thereof. In some embodiments, the parameter associated with the episode of pain comprises a physiological signal, EMG signal, ENG signal, digital signal, patient actuated signal based on perception of pain or increased pain, pressure signal, muscle contraction event signal, motion signal, orientation signal, posture signal, GPS signal, speed signal, location signal, time of day signal, time interval signal, or any combination thereof. In some embodiments, the sensor comprises a sensor electrode, receiver, pressure sensor, accelerometer, gyroscope, magnetometer, global positioning system, or any combination thereof. In some embodiments, the sensor is configured to detect activity of a muscle of the individual, and wherein the activity is associated with an EMG signal, ENG signal, digital signal, patient actuated signal based on perception of pain or increased pain, pressure signal, muscle contraction event signal, or any combination thereof.

Provided herein are systems for treating pain of an individual in need thereof, the system comprising: (a) a sensor implanted in a pelvic region of the individual configured to sense a parameter associated with an episode of pain of the individual; (b) a stimulator electrode implanted in the pelvic region of the individual configured to provide an adapted electrical stimulation; (c) a processor operably coupled to the stimulator electrode and the sensor; and (d) a non-transitory computer readable storage medium including software configured to cause the processor to: (i) receive from the sensor the parameter; (ii) analyze the parameter to determine at least one of an intensity, a frequency, or a duration of an adapted electrical stimulation, wherein the adapted electrical stimulation is configured to reduce pain of the episode; and (iii) cause the device to provide the adapted electrical stimulation with the implanted electrode, wherein the adapted electrical stimulation is provided at a first anatomical site in the pelvic area of the individual, a second anatomical site, or any combination thereof, and wherein the electrical stimulation reduces the pain of the individual. In some embodiments, the sensor, the implanted electrode, or a combination thereof, are implanted at or adjacent to a pudendal nerve, sacral nerve, pelvic plexus nerve, or any combination thereof. In some embodiments, at least one of the intensity, frequency, or duration of the adapted electrical stimulation provided in step (iii) varies according to the parameter that is detected by the sensor. In some embodiments, the stimulator electrode comprises a first stimulator and wherein the first stimulator is implanted at or adjacent to a first anatomical site. In some embodiments, the stimulator electrode further comprises a second stimulator and wherein the second stimulator is implanted at or adjacent to a second anatomical site. In some embodiments, the software further comprises providing a base electrical stimulation with the implanted stimulator electrode before the adapted electrical stimulation is provided. In some embodiments, the adapted electrical stimulation is different from the base electrical stimulation. In some embodiments, at least one of the intensity or the frequency of the adapted electrical stimulation is different from that of the base electrical stimulation. In some embodiments, the adapted electrical stimulation boosts the base electrical stimulation. In some embodiments, the adapted electrical stimulation, alone or together with the base electrical stimulation, reduces the pain of the episode. In some embodiments, the adapted electrical stimulation comprises a first stimulation pattern and a second stimulation pattern, wherein the first stimulation pattern is provided by the first stimulator and the second stimulation pattern is provided by the second stimulator. In some embodiments, the first and second stimulation patterns comprise excitation values for intensity, frequency, phase, pulse width, or any combination thereof. In some embodiments, the first and second stimulation patterns are the same. In some embodiments, the first and second stimulation patterns differ in intensity, frequency, phase, pulse width, or any combination thereof. In some embodiments, the software generates the first and second stimulation patterns based on at least the parameter. In some embodiments, the generated first and second stimulation patterns are configured to reduce pain of the episode based on at least the parameter. In some embodiments, the parameter comprises a first parameter and a second parameter and wherein the adapted electrical stimulation is further sustained, modified, redirected, withheld, or cancelled based on the second parameter and wherein the second parameter is later in time to the first parameter. In some embodiments, the sensor and the stimulator electrode are electrically coupled to a processor. In some embodiments, the parameter is provided by the individual via a controller in wireless communication with the processor. In some embodiments, the sensor is configured to transmit data to the processor, and wherein the data is associated with the parameter that is detected by the sensor. In some embodiments, the parameter associated with the episode comprises an electrical parameter, a pressure parameter, a motion parameter, or any combination thereof. In some embodiments, the parameter associated with the episode comprises a physiological signal, EMG signal, ENG signal, digital signal, patient actuated signal based on perception of pain or increased pain, pressure signal, muscle contraction event signal, motion signal, orientation signal, posture signal, GPS signal, speed signal, location signal, time of day signal, time interval signal, or any combination thereof. In some embodiments, the sensor is configured to detect the parameter. In some embodiments, the sensor comprises a sensor electrode, receiver, pressure sensor, accelerometer, gyroscope, magnetometer, global positioning system, or any combination thereof. In some embodiments, the parameter detected by the receiver, pressure sensor, accelerometer, gyroscope, magnetometer, global positioning system, or any combination thereof is associated with a pressure, muscle contraction event, motion, orientation, posture, GPS reading, speed, location, time of day, time interval, or any combination thereof. In some embodiments, the parameter detected by the sensor electrode, receiver, pressure sensor, or any combination thereof is associated with a physiological event, EMG signal, ENG signal, digital signal, patient actuated input based on perception of pain or increased pain, pressure, muscle contraction event, or any combination thereof. In some embodiments, the receiver comprises a controller receiver, digital receiver, radio receiver, or any combination thereof. In some embodiments, the sensor is configured to detect activity of a muscle of the individual, and wherein the activity is associated with an EMG signal, ENG signal, digital signal, patient actuated signal based on perception of pain or increased pain, pressure signal, muscle contraction event signal, or any combination thereof. In some embodiments, the muscle comprises a pelvic floor muscle. In some embodiments, the parameter is associated with contraction, increased tone, or any combination thereof of the pelvic floor muscle. In some embodiments, the sensor is configured to detect activity of a sensed nerve of the individual, and wherein the activity is associated with an ENG signal of the sensed nerve. In some embodiments, the sensed nerve comprises a motor nerve fiber, a larger diameter afferent nerve, or any combination thereof. In some embodiments, the sensed nerve comprises a pudendal nerve, a sacral nerve, a nerve of the pelvic plexus, or any combination thereof. In some embodiments, the episode of pain comprises a recurring pain, an exacerbating pain, or any combination thereof. In some embodiments, the pain comprises pelvic pain. In some embodiments, the pain further comprises chronic pelvic pain. In some embodiments, the pain is associated with interstitial cystitis, painful bladder syndrome, chronic prostatitis, urethral pain syndrome, vagina pain, vulvodynia, vestibulodynia, radiation vaginitis, penile pain syndrome, proctalgia fugax, anorectal pain (e.g., unspecified functional anorectal pain syndrome), phantom rectum syndrome, levator ani syndrome, pelvic floor muscle pain syndrome, pelvic floor muscle pain dysfunction, pelvic floor myalgia, urethral pain syndrome, coccygodynia, pudendal neuralgia, or any combination thereof. In some embodiments, the episode of pain of the individual occurs when the individual sits, changes posture, urinates, defecates, attempts to urinate, attempts to defecate, has sexual intercourse, or any combination thereof. In some embodiments, the parameter is associated with the individual sitting, changing posture, urinating, defecating, attempting to urinate, attempting to defecate, having sexual intercourse, or any combination thereof. In some embodiments, the pain is associated with clinical or sub-clinical inflammation. In some embodiments, the pain is associated with compression of at least one nerve ending during a striated muscle spasm. In some embodiments, the pain is associated with compression of at least one nerve ending during an anatomical compression event. In some embodiments, the pain comprises neuropathic pain. In some embodiments, the pain is associated with nerve damage or injury. In some embodiments, the pain is associated with dysregulated visceral smooth muscle activity. In some embodiments, the pain of the individual has diurnal variation. In some embodiments, the parameter comprises temporal or circadian functions. In some embodiments, the first anatomical site, the second anatomical site, or any combination thereof is adjacent to or at a nerve. In some embodiments, the nerve comprises a motor nerve fiber, a larger diameter afferent nerve, or any combination thereof. In some embodiments, the nerve gates peripheral nociception, spinal activity, or any combination thereof. In some embodiments, the nerve stimulates muscle contraction, modulates muscle activity, or any combination thereof. In some embodiments, the nerve comprises a pudendal nerve, a sacral nerve, a nerve of the pelvic plexus, or any combination thereof. In some embodiments, a pudendal nerve, a sacral nerve, a pelvic plexus nerve, or a combination thereof is electrically stimulated by the stimulator electrode. In some embodiments, stimulation is provided by the first stimulator to a first nerve before the parameter is detected and, after the parameter is detected, stimulation is provided to the first nerve, a second nerve, or any combination thereof. In some embodiments, the first anatomical site, the second anatomical site, or any combination thereof is adjacent to or at a muscle. In some embodiments, the first anatomical site, the second anatomical site, or any combination thereof is adjacent to or at a pelvic muscle. In some embodiments, the first anatomical site is adjacent to or at a first pudendal nerve, and wherein the second anatomical site is adjacent to or at a sacral nerve, the first pudendal nerve, or a second pudendal nerve. In some embodiments, a first anatomical site is adjacent to or at a first pudendal nerve, a first sacral nerve, or a first pelvic plexus nerve. In some embodiments, a second anatomical site is adjacent to or at a second pudendal nerve, a second sacral nerve, or a second pelvic plexus nerve.

Provided herein are non-transitory computer readable storage mediums including software for reducing pain of an individual in need thereof, configured to cause a processor to: (a) receive a parameter associated with an episode of pain of the individual from an implanted sensor, the implanted sensor being configured to detect the parameter; (b) analyze the parameter to determine at least one of an intensity, a frequency, or a duration of an adapted electrical stimulation; and (c) cause an implanted stimulator electrode to provide the adapted electrical stimulation to the individual, wherein the adapted electrical stimulation is configured to reduce pain of the individual. Provided herein are non-transitory computer readable storage mediums including software for reducing pain of an individual in need thereof, configured to cause a processor to: (a) cause an implanted stimulator electrode to provide a base electrical stimulation; (b) receive a parameter associated with an episode of pain of the individual from an implanted sensor, the implanted sensor being configured to detect the parameter; (c) analyze the dataset to determine at least one of an intensity, a frequency, or a duration of an adapted electrical stimulation; and (d) cause the implanted stimulator electrode to provide the adaptive electrical stimulation to the individual, wherein the adapted electrical stimulation, alone or together with the base stimulation, reduces the pain of the individual. In some embodiments, the parameter is indicative of exacerbating pain. In some embodiments, the adapted electrical stimulation is at a different intensity level than the base electrical stimulation. In some embodiments, the implanted electrode comprises a first stimulator and a second stimulator, wherein the first stimulator stimulates one region of a pudendal nerve and the second stimulator stimulates a sacral nerve, a different pudendal nerve, or a different region of the pudendal nerve. In some embodiments, the software comprises a machine learning model, and wherein the machine learning model is configured to classify the value received from the implanted sensor and generate at least one stimulation pattern configured to reduce pain of the individual when the at least one stimulation pattern is performed by the implanted electrode In some embodiments, the software further causes the processor to provide a base electrical stimulation with the implanted stimulator electrode before the adapted electrical stimulation is provided. In some embodiments, the adapted electrical stimulation is different from the base electrical stimulation. In some embodiments, at least one of the intensity or the frequency of the adapted electrical stimulation is different from that of the base electrical stimulation. In some embodiments, the adapted electrical stimulation boosts the base electrical stimulation. In some embodiments, the adapted electrical stimulation, alone or together with the base electrical stimulation, reduces the pain of the episode. In some embodiments, the adapted electrical stimulation comprises a first stimulation pattern and a second stimulation pattern, wherein the first stimulation pattern is provided by the first stimulator and the second stimulation pattern is provided by the second stimulator. In some embodiments, the first and second stimulation patterns comprise excitation values for intensity, frequency, phase, pulse width, or any combination thereof. In some embodiments, the first and second stimulation patterns are the same. In some embodiments, the first and second stimulation patterns differ in intensity, frequency, phase, pulse width, or any combination thereof. In some embodiments, the software generates the first and second stimulation patterns based on at least the parameter. In some embodiments, the generated first and second stimulation patterns are configured to reduce pain of the episode based on at least the parameter. In some embodiments, the parameter comprises a first parameter and a second parameter and wherein the adapted electrical stimulation is further sustained, modified, redirected, withheld, or cancelled based on the second parameter and wherein the second parameter is later in time to the first parameter. In some embodiments, the sensor and the stimulator electrode are electrically coupled to a processor. In some embodiments, the parameter is provided by the individual via a controller in wireless communication with the processor. In some embodiments, the sensor is configured to transmit data to the processor, and wherein the data is associated with the parameter that is detected by the sensor. In some embodiments, the parameter associated with the episode comprises an electrical parameter, a pressure parameter, a motion parameter, or any combination thereof. In some embodiments, the parameter associated with the episode comprises a physiological signal, EMG signal, ENG signal, digital signal, patient actuated signal based on perception of pain or increased pain, pressure signal, muscle contraction event signal, motion signal, orientation signal, posture signal, GPS signal, speed signal, location signal, time of day signal, time interval signal, or any combination thereof. In some embodiments, the sensor is configured to detect the parameter. In some embodiments, the sensor comprises a sensor electrode, receiver, pressure sensor, accelerometer, gyroscope, magnetometer, global positioning system, or any combination thereof. In some embodiments, the parameter detected by the receiver, pressure sensor, accelerometer, gyroscope, magnetometer, global positioning system, or any combination thereof is associated with a pressure, muscle contraction event, motion, orientation, posture, GPS reading, speed, location, time of day, time interval, or any combination thereof. In some embodiments, the parameter detected by the sensor electrode, receiver, pressure sensor, or any combination thereof is associated with a physiological event, EMG signal, ENG signal, digital signal, patient actuated input based on perception of pain or increased pain, pressure, muscle contraction event, or any combination thereof. In some embodiments, the receiver comprises a controller receiver, digital receiver, radio receiver, or any combination thereof. In some embodiments, the sensor is configured to detect activity of a muscle of the individual, and wherein the activity is associated with an EMG signal, ENG signal, digital signal, patient actuated signal based on perception of pain or increased pain, pressure signal, muscle contraction event signal, or any combination thereof. In some embodiments, the muscle comprises a pelvic floor muscle. In some embodiments, the parameter is associated with contraction, increased tone, or any combination thereof of the pelvic floor muscle. In some embodiments, the sensor is configured to detect activity of a sensed nerve of the individual, and wherein the activity is associated with an ENG signal of the sensed nerve. In some embodiments, the sensed nerve comprises a motor nerve fiber, a larger diameter afferent nerve, or any combination thereof. In some embodiments, the sensed nerve comprises a pudendal nerve, a sacral nerve, a nerve of the pelvic plexus, or any combination thereof. In some embodiments, the episode of pain comprises a recurring pain, an exacerbating pain, or any combination thereof. In some embodiments, the pain comprises pelvic pain. In some embodiments, the pain further comprises chronic pelvic pain. In some embodiments, the pain is associated with interstitial cystitis, painful bladder syndrome, chronic prostatitis, urethral pain syndrome, vagina pain, vulvodynia, vestibulodynia, radiation vaginitis, penile pain syndrome, proctalgia fugax, anorectal pain (e.g., unspecified functional anorectal pain syndrome), phantom rectum syndrome, levator ani syndrome, pelvic floor muscle pain syndrome, pelvic floor muscle pain dysfunction, pelvic floor myalgia, urethral pain syndrome, coccygodynia, pudendal neuralgia, or any combination thereof. In some embodiments, the episode of pain of the individual occurs when the individual sits, changes posture, urinates, defecates, attempts to urinate, attempts to defecate, has sexual intercourse, or any combination thereof. In some embodiments, the parameter is associated with the individual sitting, changing posture, urinating, defecating, attempting to urinate, attempting to defecate, having sexual intercourse, or any combination thereof. In some embodiments, the pain is associated with clinical or sub-clinical inflammation. In some embodiments, the pain is associated with compression of at least one nerve ending during a striated muscle spasm. In some embodiments, the pain is associated with compression of at least one nerve ending during an anatomical compression event. In some embodiments, the pain comprises neuropathic pain. In some embodiments, the pain is associated with nerve damage or injury. In some embodiments, the pain is associated with dysregulated visceral smooth muscle activity. In some embodiments, the pain of the individual has diurnal variation. In some embodiments, the parameter comprises temporal or circadian functions. In some embodiments, the first anatomical site, the second anatomical site, or any combination thereof is adjacent to or at a nerve. In some embodiments, the nerve comprises a motor nerve fiber, a larger diameter afferent nerve, or any combination thereof. In some embodiments, the nerve gates peripheral nociception, spinal activity, or any combination thereof. In some embodiments, the nerve stimulates muscle contraction, modulates muscle activity, or any combination thereof. In some embodiments, the nerve comprises a pudendal nerve, a sacral nerve, a nerve of the pelvic plexus, or any combination thereof. In some embodiments, a pudendal nerve, a sacral nerve, a pelvic plexus nerve, or a combination thereof is electrically stimulated by the stimulator electrode. In some embodiments, stimulation is provided by the first stimulator to a first nerve before the parameter is detected and, after the parameter is detected, stimulation is provided to the first nerve, a second nerve, or any combination thereof. In some embodiments, the first anatomical site, the second anatomical site, or any combination thereof is adjacent to or at a muscle. In some embodiments, the first anatomical site, the second anatomical site, or any combination thereof is adjacent to or at a pelvic muscle. In some embodiments, the first anatomical site is adjacent to or at a first pudendal nerve, and wherein the second anatomical site is adjacent to or at a sacral nerve, the first pudendal nerve, or a second pudendal nerve. In some embodiments, a first anatomical site is adjacent to or at a first pudendal nerve, a first sacral nerve, or a first pelvic plexus nerve. In some embodiments, a second anatomical site is adjacent to or at a second pudendal nerve, a second sacral nerve, or a second pelvic plexus nerve.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an exemplary embodiment of an open-loop bioelectronic system comprising an implantable pulse generator. The system delivers a predefined stimulation protocol and does not receive input from the subject.

FIG. 2 shows an exemplary embodiment of a closed-loop bioelectronic system comprising a two-way neural interface comprising a sensor that detects the neural response of the subject and a processing module that can interpret the neural response. The system can deliver an adapted stimulation based on the neural response.

FIG. 3 shows an exemplary embodiment of the devices and methods described herein targeting a pudendal nerve by placing the sensor and stimulator electrode near the pudendal nerve. The sensor captures a bio-signal associated with an episode of pain to be analyzed and classified, and the stimulator delivers an adapted electrical stimulation, wherein the adapted electrical stimulation was generated according to analyzed parameters comprising the bio-signal.

FIG. 4 shows an exemplary embodiment, wherein the bio-signal associated with the episode of pain is an electromyography (EMG) signal during a cough or sudden movement. The EMG is detected by the sensor and analyzed. A stimulator electrode delivers the adapted electrical stimulation comprising a “boost” that is adapted from a basal stimulation, wherein the adapted electrical stimulation is configured to reduce pain of the episode. The adapted electrical stimulation is terminated when a second bio-signal is detected, and the basal stimulation is restored.

FIG. 5 shows an exemplary embodiment of a system block diagram for the devices and methods described herein configured to implement slow-and fast-adapting algorithms. API is an application programming interface; MICS is the Medical Information and Communication band.

FIGS. 6A-6B show a flowchart of a method of closed-loop operation of the device of the disclosure.

FIGS. 7A-7B show an example embodiment of the patient controller module, as described in some embodiments herein.

FIG. 8 shows a flow diagram for purposeful patient contraction and manual operation of the devices and systems, as described in some embodiments herein.

FIG. 9 shows a flow diagram for the closed-loop system operation, as described in some embodiments herein.

FIG. 10 shows a flow diagram for signal processing and signal intensity threshold detection of signals associated with an episode of pain.

FIGS. 11A-11E show patient purposeful muscle contraction, Valsalva maneuver, and coughing EMG data acquired and processed with the methods of the disclosure, as described in some embodiments herein.

FIGS. 12A-12B show flow diagrams for detecting a purposeful or intent based contraction by the patient and providing electrical stimulation to reduce pain associated with an episode of pain, as described in some embodiments herein.

FIG. 13 shows a flow diagram for training on-board machine learning classifier of the devices and systems of the disclosure, as described in some embodiments herein.

### DETAILED DESCRIPTION

Chronic pelvic pain (CPP) is a complex condition and involves non-malignant pain perceived in the pelvic area for at least 6 months. CPP can impact quality of life, often causing episodes of severe discomfort and exacerbating pain. Common symptoms of CPP include but are not limited to neuropathic symptoms like paresthesia, numbness, burning, lancinating pain, in the pelvic, anus and/or genitals. CPP comprises episodes of a recurring pain, exacerbating pain, or any combination thereof.

CPP may be associated with various conditions. These conditions include but are not limited to interstitial cystitis, painful bladder syndrome, chronic prostatitis, urethral pain syndrome, vagina pain, vulvodynia, vestibulodynia, radiation vaginitis, penile pain syndrome, proctalgia fugax, anorectal pain (e.g., unspecified functional anorectal pain syndrome), phantom rectum syndrome, levator ani syndrome, pelvic floor muscle pain syndrome, pelvic floor muscle pain dysfunction, pelvic floor myalgia, urethral pain syndrome, coccygodynia, pudendal neuralgia, voiding dysfunction, and constipation. In some cases, these conditions involve the pudendal nerve. In some cases, CPP comprises pain associated with one or more of clinical or sub-clinical inflammation, compression of a nerve ending (e.g., during a striated muscle spasm or an anatomical compression event), neuropathic pain, nerve damage or injury, dysregulated visceral smooth muscle activity. Pain associated with CPP can occur when the individual sits, changes posture, urinates, defecates, attempts to urinate, attempts to defecate, and/or has sexual intercourse. Pain may have diurnal variation in the individual over the course of the day. In some cases, the pain may be exacerbated by activities.

CPP often relates to neurological issues, making neuromodulation of the central or peripheral nervous system an attractive approach to reduce pain of the affected individuals. Treatments for pain may target the entire body, pelvic area, and/or nervous system rather than specific peripheral nerves within the pelvic area. Such treatments may include providing nerve blockers and/or physical therapy to an individual to help alleviate pain in the pelvic area. Treatments involving electrical stimulation may include transcutaneous electrical nerve stimulation (TENS) therapy, which provides electrical impulses to the pelvic region through electrodes placed on the surface of the skin of the individual, with the goal of alleviating pain in the pelvic area. In some cases, spinal cord stimulation (SCS) has had a limited success in the treatment of back and/or leg pain. SCS may be somewhat limited due to the inability to consistently obtain coverage to most painful areas.

In some cases, pain usually may persist even if the organ-specific insult is over, so focused organ therapies may be inadequate. As such, neuromodulation of the peripheral nervous system may be a more attractive approach. Especially in cases where the pain areas may be well defined and can be linked to the target site of the peripheral nerve, peripheral nerve stimulation may have high efficacy in treating CPP and pain conditions. In some cases, individuals with CPP who are refractive to conservative care may responds to sacral nerve (SNM), but the success of SNM may vary, especially in patients with pudendal neuralgia. In some cases, pain associated conditions have pudendal nerve involvement. Thus, electrical stimulation of the pudendal nerve, whether alone or in combination with other nerve targets, has the potential to be an efficacious approach to reducing pain.

Many electrical nerve stimulation approaches currently used deliver a preset stimulation protocol (i.e., open-loop configuration) and usually are not able to adapt to the changing parameters of pain experienced by the individual. In some cases, the inability to adapt the stimulation can result in overstimulating or under stimulating the target area and lead to inefficient or inadequate pain management. Moreover, because treatments usually do not adapt to the changing parameters of pain, existing pain management often requires patient-actuation during bouts of exacerbating pain to provide pain relief. Therefore, it would be highly beneficial to provide electrical stimulation to peripheral nerve targets that adapt to innate feedback from the subject as conditions change (i.e., a closed-loop configuration).

Provided herein are devices, systems, and methods for preventing and/or reducing pain associated with an episode of pain in an individual in need thereof. The systems, methods, and devices, described herein are directed to treating episodes of pain associated with chronic pelvic pain (CPP) or other conditions resulting in pelvic pain using peripheral nerve stimulation. In some embodiments, the systems, methods, and devices, comprise a closed-loop configuration. In some embodiments, adapted stimulation to the target nerves are provided by an implanted stimulator with an underlying physiological rationale comprising: (a) stimulating motor fibers to alter end organ muscle activity where peripheral pain is driven by spasm and/or hypertonicity (e.g., pelvic floor myalgia, some cases of bladder pain syndrome, and urethral pain associated with motor modulation); (b) stimulating larger diameter afferent fibers to modulate spinal gating of nociceptive signaling from peripheral foci of pain generation (e.g., interstitial cystitis, coccygodynia, and pelvic myalgia); (c) blocking nerve conduction (e.g., anodal block) to (i) directly block disease-related peripherally driven pain, and (ii) block noxious effects associated with providing the adapted stimulation, which facilitates higher charge delivery for therapeutic benefit; and any combination thereof.

In some cases, the individual comprises a female subject, at least 18 years of age with chronic pelvic pain, pressure, or discomfort with moderate to severe pain criteria of greater than or equal to 4 points on the numeric pain rating scale (NPRS). In some cases, the pelvic pain, pressure or discomfort is related to the urinary bladder indicated by: a questionnaire where an individual indicates greater than or equal to one urinary symptom e.g., persistent urge to void or frequency of voiding experienced by the individual for at least 3 months. In some cases, the individual comprises an individual who has experienced a duration of chronic pelvic pain systems less than or equal to 3 months. In some instances, the individual comprises a bladder with normal bladder compliance and no Hunner's lesion(s) as defined by the American Urological Association (AUA) or the European Association of Urology (EAU). In some cases, individual comprises an individual who has failed or is not a candidate for conversative treatment e.g., pelvic floor muscle therapy, biofeedback, and/or behavioral modification.

Described herein are targeting one or more peripheral nerves based on the etiology of the pain condition with adapted electrical stimulation to reduce pain experienced by an individual. In some embodiments, the stimulator electrodes target different nerves (e.g., a first stimulator targeting a sacral nerve and a second stimulator targeting a pudendal nerve). In some embodiments, stimulating multiple nerves within the pelvic area may broaden the field of treatment in pain syndromes having diffuse areas of pain. In some embodiments, the electrical stimulation provide may be adapted to provide blocking and stimulation of electrical nerve signals on the same nerve. In some embodiments, stimulator electrodes target one or more locations along a single nerve. In some embodiments, targeting multiple points along a single nerve allows for improved control in the closed-loop modulation (e.g., a first stimulator implanted at or adjacent to a first anatomical site of a pudendal nerve and a second stimulator implanted at or adjacent to a second anatomical site of the pudendal nerve). In some embodiments, targeting a single nerve at multiple sites permits both blocking and stimulation on the same nerve.

In some embodiments, the stimulation provided by the electrodes to different anatomical sites may be the same or differ in at least one of intensity, frequency, phase, or pulse width. The stimulation provided by each stimulator may start and/or end at different times relative to the stimulations provided by another stimulator, depending on the pain detected in the individual. In some embodiments, a first stimulator provides a first stimulation pattern and a second stimulator provides a second stimulation pattern, depending on the pain detected in the individual.

In some embodiments, the electrode provides a basal stimulation to the target nerve before an adapted stimulation (e.g., an electrical stimulation having an adapted stimulation pattern) is provided. In some embodiments, the adapted electrical stimulation is provided by the same electrode that provided the basal stimulation. In some embodiments, the adapted electrical stimulation is provided by a different electrode than the one providing the basal stimulation. In some embodiments, the adapted stimulation provide at least one of: (i) a “boost” of the basal stimulation parameters (e.g., increase the amplitude of the basal stimulation); (ii) a switch to a different stimulation program (e.g., a switch from sacral to pudendal nerve stimulation or a switch from stimulating to blocking frequencies); or (iii) a change from single to dual nerve stimulation (e.g., pudendal stimulation to simultaneous pudendal, sacral and/or pelvic plexus stimulation).

Provided herein are devices and systems comprising one or more sensors, one or more stimulator electrodes, a processor, a power source, or any combination thereof. In some embodiments, the one or more stimulator electrodes, one or more sensors, a processor, a power source, or any combination thereof may be implanted into the body of the individual. In some embodiments, the device may be placed superficially on the body of the individual. In some embodiments, the device may be implanted into the body of the individual. In some embodiments, the device comprises a wireless transmission module capable of transmitting and receiving wireless data wireless with a remote device (e.g., a mobile phone, a tablet, a computer, etc.). In some embodiments, the device comprises a hermetically sealed connector placed on the individual's skin superficially that may electrically couple to a remote device by a cable. In some embodiments, the individual or a healthcare professional may modify the settings of the sensor and/or the stimulator electrode (e.g., a threshold parameter and/or signal intensity threshold). In some embodiments, the setting may be modified using a graphical user interface on the remote device. In some embodiments, the device automatically modifies the setting of the sensor and/or the stimulator electrode. In some embodiments, the device may automatically modify the setting of the sensor and/or the stimulator electrode based on a parameter detected by the sensor. In some embodiments, the setting may comprise one or more of sensitivity of the sensor or stimulator, activity of the sensor or stimulator, a length signal acquisition period, a stimulation pattern provided by a stimulator to the surrounding tissue.

The devices, systems, and methods described herein may prevent and/or reduce pain in an individual by detecting a parameter associated pain in an individual and adjusting an electrical stimulation by one or more stimulator electrodes based on the parameter's characteristics. In some embodiments, the sensor monitors parameters of the internal environment of the individual for activities associated with episodes of pain (e.g., detecting muscle function by pelvic floor EMG or afferent nerve conduction). In some embodiments, the sensor measures one or more of EMG, ENG, pressure, temperature, blood flow, acceleration, movement, orientation, 3-D spatial location, and physical deformation/stretch at or near the target tissue. In some embodiments, the sensor monitors whether anodal block associated with a basal or an adapted stimulation provided by the device has adequately reduced the pain. In some embodiments, patient actuation is used to indicate an episode of pain. In some embodiments, patient actuation is used to indicate an episode of pain associated with episodic exacerbations (e.g., pelvic floor myalgia) and/or intermittent pain (e.g., proctalgia fugax). In some embodiments, patient actuation is used to indicate an episode of postural pain associated with a CPP condition (e.g., patients with myalgia upon sitting). After the episode of pain is indicated, the electrical stimulation may be provided to reduce pain of the episode. In some embodiments, a boost to a basal stimulation pattern is provided by the adapted stimulation. In some embodiments, the sensor may detect a change in posture of the individual and/or received a patient actuated signal. In some embodiments, circadian functions are used to indicate pain having diurnal variation. In some embodiments, the diurnal variation comprises night-time pain. In some cases, pain is worse at night and/or with movement. In some embodiments, a different stimulation pattern may be provided when the individual wakes compared to when the individual sleeps.

In some embodiments, a sensor is configured to sense one or more parameters associated with an episode of pain or an attempt by the individual to reduce pain. In some embodiments, the parameter comprises an electromyography (EMG) signal associated with an episode of pain. In some embodiments, the EMG signals are associated with electrical activity of muscles, specifically from action potentials in muscle fibers. In some embodiments, the parameter comprises an electroneurogram (ENG). In some embodiments, an electroneurogram comprises electrical activity from one or more neurons and typically refers to recordings made from bundles of axons in peripheral nerves. In some embodiments, the parameter comprises a change of electric impedance caused by physical deformation of the sensor material. In some embodiments, the physical deformation comprises stretching, compression, or any combination thereof. In some embodiments, the parameter associated with an episode of pain comprises a change in the amplitude, frequency, phase, or any combination thereof one or more of EMG, ENG, accelerometer, gyroscope, magnetometer, pressure sensor signals, or any combination thereof. By providing adaptive electrical stimulation in combination to the basal stimulation and/or individual's response, the devices, systems, and methods described herein may reduce pain in the individual.

In some embodiments, the parameter comprises a change in pressure, velocity, acceleration, or 3-D spatial direction. In some embodiments, 3-D spatial direction is determined by GPS signal. The GPS signal, in some embodiments, may indicate and recognize when the individual is in proximity to locations including at least one of the individual's home, bedroom, living room, dining room, bathroom, or office. In some embodiments, the GPS signal may be configured to modulate the adapted stimulation pattern, described elsewhere herein, based on the GPS coordinates and/or GPS location of the subject. In some embodiments, the GPS signal of a location and/or GPS coordinates may indicate locations to help to predict or indicate certain activities that are commonly associated with an episode of pain. In some embodiments, the change in one or more of pressure, velocity, acceleration, or 3-D spatial direction may indicate a posture or a change in postures of the individual. In some embodiments, changes in pressure may be measured by a pressure sensor. In some embodiments, the pressure sensor comprises a differential pressure sensor, absolute pressure sensor, or any combination thereof. In some embodiments, changes in velocity, acceleration, or changes in 3-D spatial direction may be measured by an accelerometer, gyroscope, magnetometer, or any combination thereof. In some embodiments, the device provides an electrical stimulation using one or more implanted stimulator electrodes that, alone or together with a basal electrical stimulation, reduces pain of an episode. In some embodiments, modulating the adapted stimulation pattern comprises adjusting a detection parameter (e.g., signal intensity threshold) of the classifier, described elsewhere herein.

In some embodiments, the trigger to start the adapted electrical stimulation is a parameter detected by the sensor. In some embodiments, the trigger to start the adapted electrical stimulation is associated with patient actuation (e.g., sending a signal by the patient to the sensor prior to changing posture, during episodes of pain exacerbation). In some embodiments, the trigger to start the adapted electrical stimulation is a parameter associated with pelvic muscle contraction (e.g., EMG signal and/or ENG signal). In some embodiments, at least one of the basal or adapted electrical stimulation stops being provided when the sensor receives a change in the detected parameter (e.g., a patient actuated signal after the patient finishes urinating or change posture). In some embodiments, the basal and/or adapted stimulation patterns have a finite duration. In some embodiments, the duration of the basal and/or adapted stimulation patterns are determined by the detected parameter. In some embodiments, the basal and/or adapted stimulation patterns last until a change in the detected parameter above or below a threshold.

In some embodiments, the device may be implanted in the individual in proximity to the pudendal nerve, a sacral nerve, a nerve of the pelvic plexus, or branches thereof. The pelvic plexus may comprise one or more nerves that that innervate tissue involved with urination and/or defecation in a pelvis of an individual. In some instances, one or more nerves of the pelvic plexus may comprise the splanchnic nerve, hypogastric nerve, autonomic plexus nerve, or any combination thereof nerves. The pelvic plexus may comprise one or more parasympathetic and/or sympathetic nerves. In some embodiments, the one or more stimulator electrodes may be placed at or near the pudendal nerve, the sacral nerve, and/or a nerve of the pelvic plexus. In some embodiments, the one or more stimulator electrodes or one or more sensors may be implanted in proximity to a pudendal, sacral, and/or pelvic plexus nerve unilaterally or bilaterally. In some embodiments, the one or more stimulator electrodes or one or more sensors may be implanted unilaterally or bilaterally based at least on the pain or pain episode the individual has experienced or may experience. For example, an individual experiencing unilateral pain may be treated with one or more electrodes implanted at or adjacent to a single nerve, whereas an individual experiencing bilateral diffuse pain may be treated with one or more electrodes implanted at or adjacent to one or more different nerves. In some cases, the one or more stimulator electrodes and/or one or more sensors may be implanted unilaterally at or adjacent a first region of a nerve. In some embodiments, the one or more stimulator electrodes and/or one or more sensors may be implanted bilaterally at or adjacent to a first region nerve and a second region of a nerve where the first and second region are spatially independent regions of the nerve. In some cases, unilateral pain may be treated by implanting one or more electrode leads (e.g., stimulator electrode and sensor electrode) at or adjacent to a trunk and distal pudendal nerve, pudendal and sacral nerve, pudendal and pelvic autonomic nerve, or any combination thereof. In some instances, bilateral pain may be treated by implanting one or more electrode leads (e.g., stimulator electrode and sensor electrode) at or adjacent the pudendal nerve bilaterally, the sacral nerve bilaterally, pelvic autonomic nerve bilaterally. In some embodiments, the one or more stimulator electrodes may be implanted to stimulate motor nerve fibers (e.g., to the pelvic floor). In some embodiments, the one or more stimulator electrodes may be implanted within, proximate, or adjacent to the muscle (e.g., pelvic floor). The implanted sensor may sense a signal that indicates that an individual may exhibit an episode of pain. The device may analyze the signal and classify the signal as a real-time or prospective episode of pain. The device may generate an electrical stimulation that is modulated based on the classified episode of pain and may deliver the adapted electrical stimulation using one or more stimulator electrodes to the target nerve. In some embodiments, the modulation of the electrical stimulation comprises changing the frequency, amplitude, and/or pulse width of electric stimulation. In some embodiments, the modulation of the electrical stimulation comprises a modulation in simulator configuration. In some embodiments, the one or more electrodes comprises one or more stimulation electrodes and one or more sensing electrodes. The one or more stimulation electrodes and one or more sensing electrodes may be configured to switch between stimulating and sensing operations on demand, programmatically, user controlled, medical personal control, or any combination thereof. The electrical stimulation may be modulated to improve the muscle and/or nerve response to prevent or reduce the episode of pain. The modulated electrical stimulation may result in improved muscle response, as measured by response time, muscle function, or other markers of prevention or reduction of pain, to prevent the potential episode of pain. The electrical stimulation may be modulated to reduce a severity and/or duration of an episode of pain. In some embodiments, the electrical stimulation may reduce the intensity or duration of pain.

In some embodiments, the electrical stimulation reduces the duration and/or severity of the episode of pain as compared without the electrical stimulation. In some embodiments, the electrical stimulation reduces or inhibits a muscle contraction, spasm, twitch, or any combination thereof. In some embodiments, the muscle contraction, spasm, or twitch occurs before or during the episode of pain. In some embodiments, the electrical stimulation comprises the basal and adapted electrical stimulation. In some embodiments, the electrical stimulation comprises the basal stimulation. In some embodiments, the electrical stimulation comprises the adapted electrical stimulation.

In some embodiments, the stimulator electrode provides a basal electrical stimulation, an adapted electrical stimulation, or any combination thereof. In some embodiments, the electrical stimulation comprises an electrical stimulation provided over a period of time. In some embodiments, the period of time comprises the period of time a subject has a muscle contraction, spasm, twitch, or any combination thereof. In some embodiments, the muscle contraction, spasm, or twitch triggers EMG threshold detection. In some embodiments, the period of time for the electrical stimulation comprises about 1 second to about 30 seconds. In some embodiments, the period of time for the electrical stimulation comprises about 1 second to about 5 seconds, about 1 second to about 10 seconds, about 1 second to about 15 seconds, about 1 second to about 20 seconds, about 1 second to about 25 seconds, about 1 second to about 30 seconds, about 5 seconds to about 10 seconds, about 5 seconds to about 15 seconds, about 5 seconds to about 20 seconds, about 5 seconds to about 25 seconds, about 5 seconds to about 30 seconds, about 10 seconds to about 15 seconds, about 10 seconds to about 20 seconds, about 10 seconds to about 25 seconds, about 10 seconds to about 30 seconds, about 15 seconds to about 20 seconds, about 15 seconds to about 25 seconds, about 15 seconds to about 30 seconds, about 20 seconds to about 25 seconds, about 20 seconds to about 30 seconds, or about 25 seconds to about 30 seconds. In some embodiments, the period of time comprises about 1 second, about 5 seconds, about 10 seconds, about 15 seconds, about 20 seconds, about 25 seconds, or about 30 seconds. In some embodiments, the period of time for the electrical stimulation comprises at least about 1 second, about 5 seconds, about 10 seconds, about 15 seconds, about 20 seconds, or about 25 seconds. In some embodiments, the period of time for the electrical stimulation comprises at most about 5 seconds, about 10 seconds, about 15 seconds, about 20 seconds, about 25 seconds, or about 30 seconds.

### Adapted Electrical Stimulation

The disclosure describes devices for preventing or reducing pain of an individual comprising one or more sensors and one or more stimulator electrodes. In some embodiments, the device further comprises a processor, memory, a user interface, a power source, or any combination thereof for preventing or reducing an episode of pain. The devices may be implantable. The surgical procedure to implant the device may be completed under awake sedation, general anesthesia, local anesthesia, twilight anesthesia, or any combination thereof. The devices may be implanted wholly or partly in an individual's pelvic region. In some embodiments, the devices may be implanted by one or more surgical instruments. In some embodiments, surgical instruments comprise introducers, sheaths, directable probes, wires, needles, or any combination thereof.

In some embodiments, the devices further comprise a transmitter electrically coupled to a processor capable of wirelessly transmitting and receiving data from a remote device, such as a mobile phone, a tablet, or a computer. In some embodiments, the device may be configured for open-loop configuration. In some embodiments, the device may be configured for a close-loop or feedback-controlled configuration.

Aspects of the disclosure provided herein comprise methods of data processing. In some embodiments, the method of data processing comprises: (a) receiving a parameter associated with an episode of pain of the individual from an implanted sensor, the implanted sensor being configured to detect the parameter; (b) analyzing the parameter, comprising classifying the parameter against a comparative parameter of a dataset; and (c) generating a stimulation pattern configured to reduce pain of the episode when the stimulation pattern is performed by an implanted electrode, wherein at least one of an intensity, a frequency, or a duration of the stimulation pattern varies according to analysis of the parameter.

In some embodiments, the parameter comprises a bio-signal, extrinsic signal, learned signal, or any combination thereof. In some embodiments, the dataset comprises data generated by a user, a subject, a population, or any combination thereof. In some embodiments, the comparative parameter comprises an innate value, extrinsic value, learned value, or any combination thereof. In some embodiments, software configured to generate the first and second stimulation patterns is used. In some embodiments, the software comprises a machine learning model, and wherein the machine learning model is configured to classify the parameter received by the implanted sensor and generate the stimulation patterns. In some embodiments, the machine learning model comprises training a classifier of user-specific activity based on at least one of a GPS reading, time of day, or motion. In some embodiments, the comparative parameter comprises a bio-signal, extrinsic signal, learned signal, or any combination thereof.

### Open-Loop Configuration of the Device

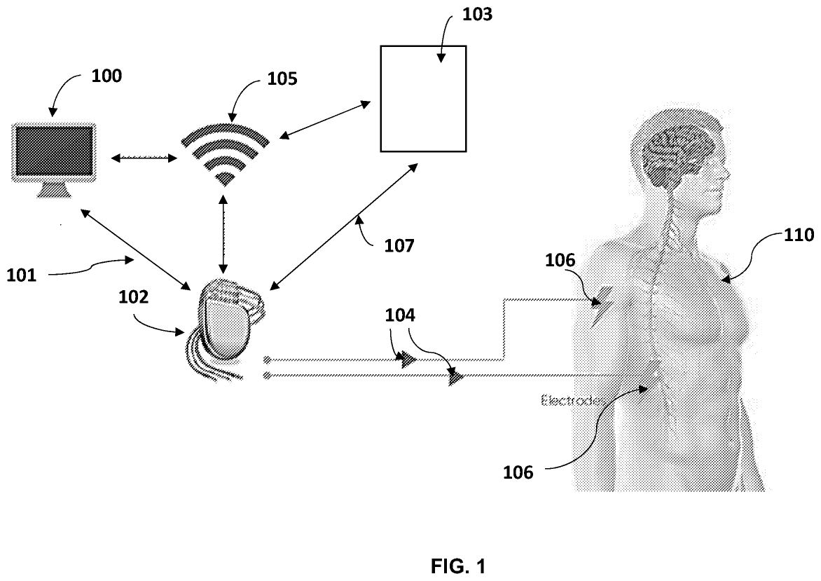

FIG. 1 shows an open-loop configuration of the device described herein configured to prevent or reduce pain in a pelvic region of an individual 110. The device in an open-loop configuration comprises an implantable pulse generator 102 comprising one or more stimulator electrodes 106, and a power source. In some embodiments, the implantable pulse generator 102 comprises a processor, and a wireless transmission module configured to execute software to administer an electrical stimulation pattern 104. In some embodiments, the power source comprises a battery. In some embodiments, the battery may be a lithium polymer ion battery, lithium iodine, lithium manganese dioxide, lithium carbon monofluoride, or any combination thereof. In some embodiments, the battery may be wirelessly charged by an inductive charger. In some embodiments, the battery power source may be a single use.

The implantable pulse generator 102 may deliver a predefined electrical stimulation pattern 104 that has been set by a healthcare provider on a remote device 100 (e.g., a mobile phone, a tablet, a computer, etc.) and transmitted wirelessly 105 to the implantable pulse generator 102. In some embodiments, the implantable pulse generator 102 may deliver a predefined electrical stimulation pattern 104 that has been set by a healthcare provider on a remote computing device 100 (e.g., a mobile phone, a tablet, a computer, etc.) and transmitted via a wired communication 101 to the implantable pulse generator 102. In some embodiments, the healthcare provider may set the predefined electrical stimulation parameters through a graphical user interface on the remote device. In some embodiments, an implantable pulse generator 102 implanted in the pelvic region of the individual 110 may modify or set electrical stimulation parameters via an external input device 103 (e.g., a mobile phone, a tablet, a computer, etc.) via a wireless communication 105 of the implantable pulse generator 102. In some embodiments, an implantable pulse generator 102 implanted in the pelvic region of the individual 110 may modify or set electrical stimulation parameters via an external input device 103 via a wired communication 107 of the implantable pulse generator 102. In some embodiments, an implantable pulse generator 102 implanted in the pelvic region of the individual 110 may adjust the electrical stimulation pattern 104 using a graphical user interface on the external input device 103. In some embodiments, the electrical stimulation pattern 104 parameters that may be adjusted comprise frequency, amplitude, pulse width, or any combination thereof.

### Closed-Loop Configuration of the Device

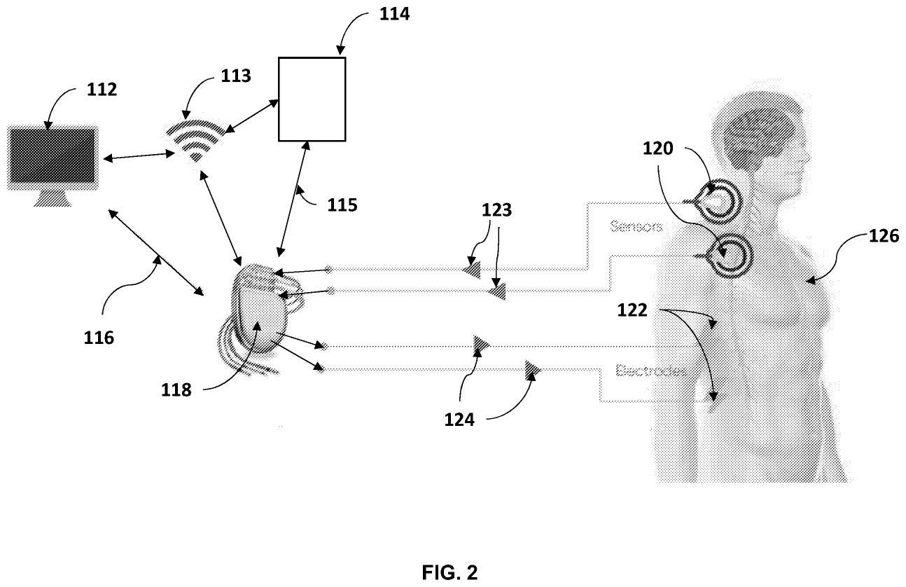

FIG. 2 shows a closed-loop configuration of the device described herein configured to prevent or reduce pain of an individual 126. The device in a closed-loop configuration comprises an implantable pulse generator 118, one or more stimulator electrodes 122, one or more sensors 120, and a power source. In some embodiments, the implantable pulse generator 118 comprises a processor, and a wireless transmission module configured to execute software to detect, analyze myoelectric electromyograph (EMG) signals via one or more sensors 120 and administer an electrical stimulation pattern 124. In some embodiments, the power source comprises a battery. The battery may be rechargeable or single use. In some embodiments, the battery may be charged through inductive charging. In some embodiments, the device configured in a closed-loop configuration may measure EMG signals via one or more sensors 120 to detect one or more parameters associated with an episode of pain of the individual or a level of innate myoelectric electrical activity. In some embodiments the device configured in a closed-loop configuration may measure inertial signals such as rapid acceleration, shock, posture-orientation, or any combination thereof via the one or more sensors 120 to detect one or more parameters associated with an episode of pain. In some embodiments, the one or more parameters associated with the episode of pain comprise signals from actions including coughing, sneezing, laughing, or exercise. Once detected, the implantable pulse generator 118 may provide an adapted electrical stimulation pattern 124 to reduce pain of the individual based on the one or more parameters. In some embodiments, the threshold level for detecting parameters may be modified and adjusted by the individual 126 via graphical user interface on an external input device 114 either via wireless communication 113 or a wired connection 115.

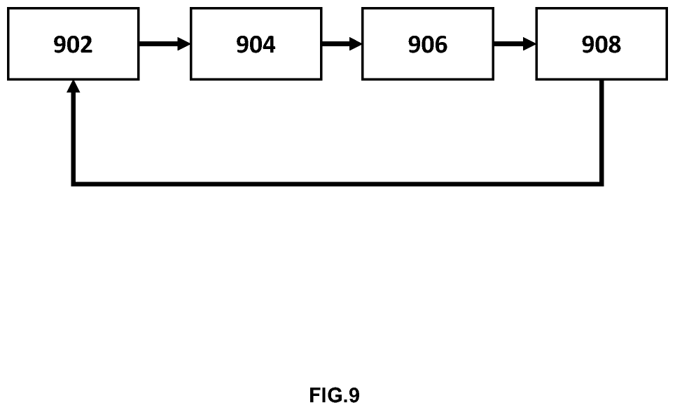

In some embodiments, a closed-loop configuration of the systems and methods described herein, comprises a fully synchronized system, as shown in FIG. 9. In some embodiments, an electrode 902, comprising a sensor and/or stimulator electrode may detect an EMG, ENG, and/or pressure signal through circuitry 904 (e.g., analog to digital circuitry) and then may pass the detected signal to a classifier algorithm 906. Once the classifier algorithm 906 has classified the detected EMG, ENG, and/or pressure signal as an episode of pain, the classifier may enable an electric stimulation 908 to be delivered to the patient. As shown in FIG. 9, the electrical stimulation may be provided by the same electrode 902 that sensed the EMG and/or ENG signal initially.

In some embodiments, a closed-loop configuration of the device described herein may be configured to detect an episode of pain in an individual. In some embodiments, the methods and systems described here may supplement the patient's effort to reduce pain with an electrical stimulation pattern via one or more stimulator electrodes 122 sufficient to reduce or prevent an episode of pain. In some embodiments, an individual's effort may be measured with an EMG, ENG, pressure, acceleration, gyroscope, magnetometer, 3-D spatial, or any combination thereof signal at a threshold. In some embodiments, the threshold myoelectric signal may be detected by one or more sensors 120. In some embodiments, the adapted stimulation, comprises an electrical signal provided by the stimulator electrodes, described elsewhere herein, with parameters e.g., frequency, pulse width, and/or amplitude such that, alone or in combination with the detected individual's effort and/or basal stimulation, may reduce prevent an episode of pain. In some embodiments the one or more parameters of the adapted stimulation may be determined on an individual subject basis and/or on a large-scale population of subjects with similar presentation. For example, for a given subject's adapted stimulation, one or more parameters may be tuned and/or determined by whether such adapted stimulation reduces an episode of pain in real-time or after the episode of pain through the sensor or a user interface of the device, described elsewhere herein. In some embodiments, for a given subject's adapted stimulation, one or more parameters may be tuned to values and/or parameters found to reduce or prevent pain in subjects with similar clinical presentation (e.g., age, type of pain, frequency of pain episode, other subject clinical meta data, etc.).

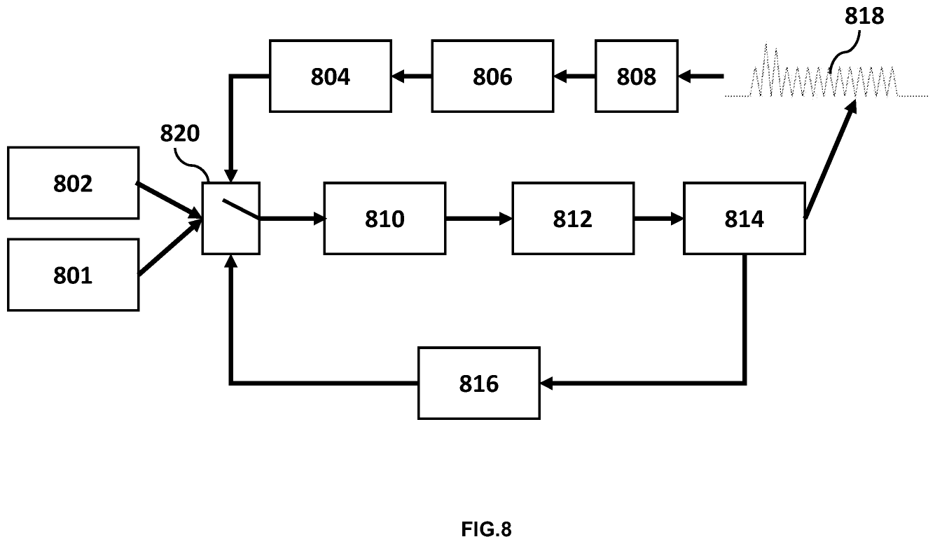

In some embodiments, the system and methods described herein comprises system and methods configured to provide an electrical stimulation to prevent an episode of pain based on a subject and/or patient's purposeful muscle contraction and/or movement, as seen in FIG. 8. In some embodiments, the subject and/or patient 814 may, upon realizing that they may exhibit an episode of pain, induce movement and/or contraction of one or more muscles or muscle groups to trigger an EMG, ENG, pressure, acceleration, gyroscope, magnetometer, 3-D spatial, or any combination thereof signal 818. In some embodiments, the induced movement and/or contraction of one or more muscle groups may be amplified 808, classified (by a classifier) 806, passed through a control logic algorithm 804, and used as a trigger 820 to enable the flow of therapy and respective basal 802 and/or active 801, described elsewhere herein, stimulation pattern parameters through the stimulator 810 and neural interface 812 to prevent an episode of pain. In some embodiments, the classifier comprises a machine learning classifier. In some embodiments, the classifier comprises an intensity threshold classifier, described elsewhere herein. In some embodiments, the patient and/or subject 814 may manually 816 enable the delivery of electrical stimulation via a button on the patient controller module 156, described elsewhere herein.

In some embodiments, the detection threshold of the implantable pulse generator 118 (i.e., stimulator) may be modified and tuned via a graphical user interface on an external input device 114. The external input device 114 may be able to modify and tune the threshold of the implantable pulse generator 118 via a wireless communication 113 or a wired connection 115. In some embodiments, the implantable pulse generator threshold may be tuned via a healthcare provider on a remote computing device 112 (e.g., a mobile phone, a tablet, a computer, etc.) and transmitted via a wired communication 116 to the implantable pulse generator 118. In some embodiments, the implantable pulse generator threshold may be tuned via a healthcare provider on a remote computing device 112 (e.g., a mobile phone, a tablet, a computer, etc.) and transmitted via wireless communication 113 to the implantable pulse generator 118. In some embodiments, the implantable pulse generator threshold may be tuned via the individual on a remote computing device 112 (e.g., a mobile phone, a tablet, a computer, etc.) and transmitted via a wired communication 116 to the implantable pulse generator 118. In some embodiments, the implantable pulse generator threshold may be tuned via the individual on a remote computing device 112 (e.g., a mobile phone, a tablet, a computer, etc.) and transmitted via wireless communication 113 to the implantable pulse generator 118.

In some embodiments, the electrical stimulation pattern 124 provided via one or more stimulator electrodes 122 may be tuned and adjusted by the detected threshold level to supplement an individual's effort to prevent an episode of pain. In some embodiments, the adjusted electrical stimulation pattern 124 may be determined by mapping a detectable physiological signal representing effort and a provided electrical stimulation pattern 124 by, piecewise linear mapping, linear mapping, sigmoidal mapping, or any variations thereof. The electric stimulation pattern 124 may be tuned by modifying or changing the electrical stimulation pattern parameters comprising frequency, pulse-width, and amplitude. In some embodiments, the electrical stimulation pattern parameters of the implantable pulse generator 118 may be modified and tuned via a graphical user interface on an external input device 114. The external input device 114 may be able to modify and tune the electrical stimulation pattern parameters of implantable pulse generator 118 via a wireless communication 113 or a wired connection 115. In some embodiments, the electrical stimulation pattern parameters of implantable pulse generator 118 may be tuned via a healthcare provider on a remote computing device 112 (e.g., a mobile phone, a tablet, a computer, etc.) and transmitted via a wired communication 116 to the implantable pulse generator 118. In some embodiments, the electrical stimulation pattern parameters of implantable pulse generator 118 may be tuned via a healthcare provider on a remote computing device 112 (e.g., a mobile phone, a tablet, a computer, etc.) and transmitted via wireless communication 113 to the implantable pulse generator 118. In some embodiments, the electrical stimulation pattern parameters of implantable pulse generator 118 may be tuned by a machine learning model executed by the processor of the implantable pulse generator 118 based on input from the individual 126. In some embodiments, the machine learning model may be configured to determine a whether or not a subject is at risk of unwanted micturition and/or fecal defecation based on muscle EMG signals detected by the one or more sensors. In some embodiments, the machine learning model may be trained to determine the presence or lack thereof an individual's effort, described elsewhere herein. For example, the machine learning model may be trained with one or more EMG signals characteristic of a subject's muscle contractions in particular EMG signals that lead to pain.

In some embodiments, the electrical stimulation pattern parameters, as described elsewhere herein (e.g., frequency, amplitude, etc.), may be set or determined by a stimulation machine learning model. In some embodiments, the stimulation machine learning model comprises a Bayesian optimization model. In some embodiments, the stimulation machine learning model may be trained with stimulation patterns that users of the devices and systems, described elsewhere herein, indicate as inhibiting episode of pain for types and/or subtypes of pain. In some embodiments, the stimulation machine learning model may be trained with the patient's type of pain. The stimulation machine learning algorithms may be trained in a cloud computing network and/or server in communication with the implantable and user-devices, described elsewhere herein, and re-distributed or downloaded to one or more users and/or patients. In effect users and/or patients may update and/or download new updates to the software of the devices and systems described herein. This aspect of the invention described herein provides an unexpected result of a patient specific and optimized electrical stimulation signal that would otherwise not be achievable with a traditional stimulator.

One or more machine learning algorithms may be used to construct the machine learning model, such as support vector machines that deploy stepwise backwards parameter selection and/or graphical models, both of which may have advantages of inferring interactions between parameters. For example, machine learning algorithms or other statistical algorithms may be used such as alternating decision trees (ADTree), decision stumps, functional trees (FT), logistic model trees (LMT), logistic regression, random forests (rf), receiver operational characteristic curves (ROC), linear regression, extreme gradient boosting (xgb), classification and regression trees, support vector machines (SVM), generalized additive model using splines (e.g., gamSpline), glmnet, multivariate adaptive regression splint (earth), neural network, k-means clustering, or any machine learning algorithm or statistical algorithm known in the art. One or more algorithms may be used together to generate an ensemble method, wherein the ensemble method may be optimized using a machine learning ensemble meta-algorithm such as boosting (e.g., AdaBoost, LPBoost, TotalBoost, BrownBoost, MadaBoost, LogitBoost, etc.) to reduce bias and/or variance.

In some embodiments, the machine learning algorithm comprises a constrained machine learning algorithm configured to run on micro-processors. In some embodiments, the machine learning algorithm comprises a machine learning algorithm operating on within a TinyML framework. In some embodiments, the machine learning algorithm, described elsewhere herein may be trained offline. The offline training may be completed on a server, cloud, or other dedicated computing clusters. In some embodiments, the offline trained machine learning algorithm could then be downloaded, deployed, and/or imported into the device to iteratively improve upon the device and system performance in preventing or reducing pain of an individual.

One of ordinary skill would realize that such an off-line training structure is feasible and realized in related but different implementation. For example, such a machine learning architecture may be utilized in performing “wake up-word” text classification that are commonly seen in smartphone devices (e.g., “hey siri”, “okay google”, etc.). In some embodiments, the machine learning algorithms described herein may operate within a framework similar to the “wake up-words” speech machine learning classifier. In some embodiments, the machine learning algorithms described herein may operate on processing power and memory allocation determined to be sufficient for “wake up” speech machine learning classifiers.

In some embodiments, the software, described elsewhere herein, may be executed by a processor located on the implanted stimulator. In some embodiments, the software located on the implanted stimulator may utilize a TinyML constrained machine learning model to accommodate the processing and memory parameters of the implanted stimulator. In some embodiments, the software may be executed offline on a cloud-based computing and/or dedicated computing cluster(s). In some embodiments, the offline processing workflow may include a high-speed (Bluetooth, Wi-Fi, medical implant communication systems, etc.) data transfer between the implanted stimulator and a local personal computing device (smartphone, tablet, laptop, etc.). The personal processing device may then communicate the implanted stimulator data to a one or more cloud and/or computer clusters that will then send back a resulting output, command, and/or notification to the device. In some embodiments, the command and/or notification comprises a warning, alert, initiation of electrical stimulation, or any combination thereof. In some embodiments, the command comprises the output of a machine learning classifier configured to determine a threshold intensity of EMG and/or ENG signals indicative of an episode of pain.

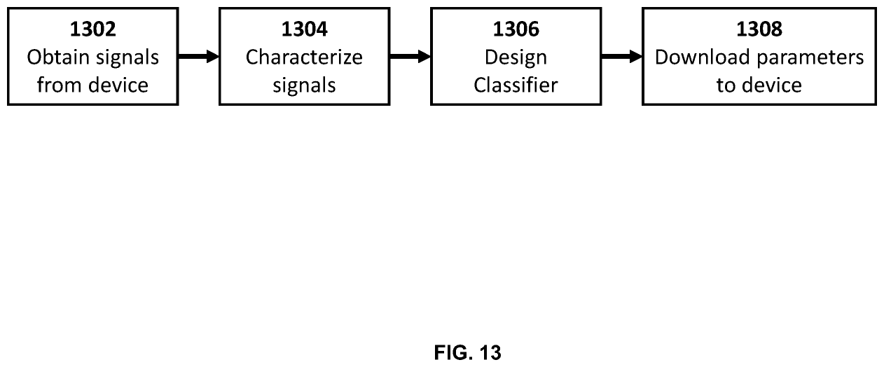

The machine learning models may be trained on one or more datasets. In some embodiments, the one or more datasets comprises data generated by a user and/or subject, or data generated by a population or segment thereof. In some embodiments, the data generated by subject and/or the data generated by a population comprises effort signals, excitation signals, that indicated an episode of pain, and prevented an episode of pain, respectively. In some embodiments, the devices, systems, and corresponding methods described herein, may record user data and/or input of the user when interacting with the systems and devices described elsewhere herein. In some embodiments, the data comprises user labeled EMG, ENG, accelerometer, gyroscope, or any combination thereof sensors, as described elsewhere herein, that lead to an episode of pain. In some embodiments, these signals may be obtained from the device 1302, and used in characterizing 1304 and training a machine learning classifier 1306, as seen in FIG. 13. In some embodiments, the trained machine learning classifiers trained on or more datasets may then be downloaded to each patient's device 1308 to further improve the machine learning classifier's accuracy. In some embodiments, the machine learning models may be configured to sense subject effort and/or providing sufficient excitation based on e.g., parameters of frequency, amplitude, and/or pulse-width as described elsewhere herein. In some embodiments, the datasets of one or more individuals may be pooled together as a training dataset where the subjects show characteristics of similarity between clinical presentation and parameters of excitatory/sensory input. In some embodiments, clinical presentation comprises clinical pain type, subject clinical meta data, e.g., gender, age, past medical history, current medications taken, past surgical intervention, etc. In some embodiments, a pooled training datasets may be utilized for an individual during the initial period of training a device implanted into a subject.

In some embodiments, the one or more machine learning models, described elsewhere herein, may be trained on raw and/or processed signals measured by the devices, sensors, and systems, described elsewhere herein. In some embodiments, the processed signals comprises original raw signals that have been filtered to optimize the signal-to-noise ratio of the raw signal. In some embodiments, the filter comprises a high-pass, low-pass, band-pass, notch, or any combination thereof filters. In some embodiments, the one or more machine learning models may be trained on user feedback regarding prior excitation signal parameters and whether or not such excitation signal parameters prevented an episode of pain.

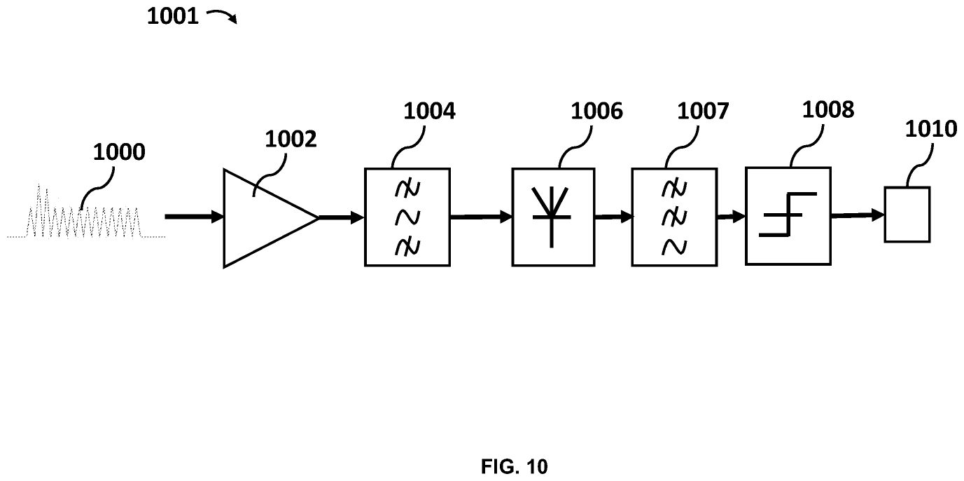

In some embodiments, the disclosure provides a method of processing detected signals 1001, described elsewhere herein to determine episode of pain precursor signal intensity thresholds, as seen in FIG. 10. In some embodiments, the EMG, ENG, accelerometer, gyroscope, magnetometer, pressure sensor signals, or any combination thereof signals 1000 may be detected through an amplifier circuit 1002. In some embodiments, the amplifier circuit comprises an operational amplifier circuit.

In some embodiments, the amplifier circuit may be configured to amplify signals from about 10 microvolt (μV) to about 1,000 μV. In some embodiments, the amplifier circuit may be configured to amplify signals from about 10 μV to about 50 μV, about 10 μV to about 100 μV, about 10 μV to about 150 μV, about 10 μV to about 300 μV, about 10 μV to about 500 μV, about 10 μV to about 700 μV, about 10 μV to about 900 μV, about 10 μV to about 1,000 μV, about 50 μV to about 100 μV, about 50 μV to about 150 μV, about 50 μV to about 300 μV, about 50 μV to about 500 μV, about 50 μV to about 700 μV, about 50 μV to about 900 μV, about 50 μV to about 1,000 μV, about 100 μV to about 150 μV, about 100 μV to about 300 μV, about 100 μV to about 500 μV, about 100 μV to about 700 μV, about 100 μV to about 900 μV, about 100 μV to about 1,000 μV, about 150 μV to about 300 μV, about 150 μV to about 500 μV, about 150 μV to about 700 μV, about 150 μV to about 900 μV, about 150 μV to about 1,000 μV, about 300 μV to about 500 μV, about 300 μV to about 700 μV, about 300 μV to about 900 μV, about 300 μV to about 1,000 μV, about 500 μV to about 700 μV, about 500 μV to about 900 μV, about 500 μV to about 1,000 μV, about 700 μV to about 900 μV, about 700 μV to about 1,000 μV, or about 900 μV to about 1,000 μV. In some embodiments, the amplifier circuit may be configured to amplify signals from about 10 μV, about 50 μV, about 100 μV, about 150 μV, about 300 μV, about 500 μV, about 700 μV, about 900 μV, or about 1,000 μV. In some embodiments, the amplifier circuit may be configured to amplify signals from at least about 10 μV, about 50 μV, about 100 μV, about 150 μV, about 300 μV, about 500 μV, about 700 μV, or about 900 μV. In some embodiments, the amplifier circuit may be configured to amplify signals from at most about 50 μV, about 100 μV, about 150 μV, about 300 μV, about 500 μV, about 700 μV, about 900 μV, or about 1,000 μV.

In some embodiments, the amplifier circuit may be configured to amplify signals with a frequency of about 1 Hz to about 1,500 Hz. In some embodiments, the amplifier circuit may be configured to amplify signals with a frequency of about 1 Hz to about 20 Hz, about 1 Hz to about 40 Hz, about 1 Hz to about 80 Hz, about 1 Hz to about 100 Hz, about 1 Hz to about 150 Hz, about 1 Hz to about 200 Hz, about 1 Hz to about 250 Hz, about 1 Hz to about 500 Hz, about 1 Hz to about 750 Hz, about 1 Hz to about 1,000 Hz, about 1 Hz to about 1,500 Hz, about 20 Hz to about 40 Hz, about 20 Hz to about 80 Hz, about 20 Hz to about 100 Hz, about 20 Hz to about 150 Hz, about 20 Hz to about 200 Hz, about 20 Hz to about 250 Hz, about 20 Hz to about 500 Hz, about 20 Hz to about 750 Hz, about 20 Hz to about 1,000 Hz, about 20 Hz to about 1,500 Hz, about 40 Hz to about 80 Hz, about 40 Hz to about 100 Hz, about 40 Hz to about 150 Hz, about 40 Hz to about 200 Hz, about 40 Hz to about 250 Hz, about 40 Hz to about 500 Hz, about 40 Hz to about 750 Hz, about 40 Hz to about 1,000 Hz, about 40 Hz to about 1,500 Hz, about 80 Hz to about 100 Hz, about 80 Hz to about 150 Hz, about 80 Hz to about 200 Hz, about 80 Hz to about 250 Hz, about 80 Hz to about 500 Hz, about 80 Hz to about 750 Hz, about 80 Hz to about 1,000 Hz, about 80 Hz to about 1,500 Hz, about 100 Hz to about 150 Hz, about 100 Hz to about 200 Hz, about 100 Hz to about 250 Hz, about 100 Hz to about 500 Hz, about 100 Hz to about 750 Hz, about 100 Hz to about 1,000 Hz, about 100 Hz to about 1,500 Hz, about 150 Hz to about 200 Hz, about 150 Hz to about 250 Hz, about 150 Hz to about 500 Hz, about 150 Hz to about 750 Hz, about 150 Hz to about 1,000 Hz, about 150 Hz to about 1,500 Hz, about 200 Hz to about 250 Hz, about 200 Hz to about 500 Hz, about 200 Hz to about 750 Hz, about 200 Hz to about 1,000 Hz, about 200 Hz to about 1,500 Hz, about 250 Hz to about 500 Hz, about 250 Hz to about 750 Hz, about 250 Hz to about 1,000 Hz, about 250 Hz to about 1,500 Hz, about 500 Hz to about 750 Hz, about 500 Hz to about 1,000 Hz, about 500 Hz to about 1,500 Hz, about 750 Hz to about 1,000 Hz, about 750 Hz to about 1,500 Hz, or about 1,000 Hz to about 1,500 Hz. In some embodiments, the amplifier circuit may be configured to amplify signals with a frequency of about 1 Hz, about 20 Hz, about 40 Hz, about 80 Hz, about 100 Hz, about 150 Hz, about 200 Hz, about 250 Hz, about 500 Hz, about 750 Hz, about 1,000 Hz, or about 1,500 Hz. In some embodiments, the amplifier circuit may be configured to amplify signals with a frequency of at least about 1 Hz, about 20 Hz, about 40 Hz, about 80 Hz, about 100 Hz, about 150 Hz, about 200 Hz, about 250 Hz, about 500 Hz, about 750 Hz, or about 1,000 Hz. In some embodiments, the amplifier circuit may be configured to amplify signals with a frequency of at most about 20 Hz, about 40 Hz, about 80 Hz, about 100 Hz, about 150 Hz, about 200 Hz, about 250 Hz, about 500 Hz, about 750 Hz, about 1,000 Hz, or about 1,500 Hz.

In some embodiments, the amplified signal is passed to a filter 1004. In some embodiments, the filter comprises a low-pass, high-pass, band-pass, notch, or any combination thereof filter.

In some embodiments, the filter may be configured to filter the frequency band of about 1 Hz to about 70 Hz. In some embodiments, the filter may be configured to filter the frequency band of about 1 Hz to about 5 Hz, about 1 Hz to about 10 Hz, about 1 Hz to about 15 Hz, about 1 Hz to about 20 Hz, about 1 Hz to about 25 Hz, about 1 Hz to about 40 Hz, about 1 Hz to about 50 Hz, about 1 Hz to about 60 Hz, about 1 Hz to about 70 Hz, about 5 Hz to about 10 Hz, about 5 Hz to about 15 Hz, about 5 Hz to about 20 Hz, about 5 Hz to about 25 Hz, about 5 Hz to about 40 Hz, about 5 Hz to about 50 Hz, about 5 Hz to about 60 Hz, about 5 Hz to about 70 Hz, about 10 Hz to about 15 Hz, about 10 Hz to about 20 Hz, about 10 Hz to about 25 Hz, about 10 Hz to about 40 Hz, about 10 Hz to about 50 Hz, about 10 Hz to about 60 Hz, about 10 Hz to about 70 Hz, about 15 Hz to about 20 Hz, about 15 Hz to about 25 Hz, about 15 Hz to about 40 Hz, about 15 Hz to about 50 Hz, about 15 Hz to about 60 Hz, about 15 Hz to about 70 Hz, about 20 Hz to about 25 Hz, about 20 Hz to about 40 Hz, about 20 Hz to about 50 Hz, about 20 Hz to about 60 Hz, about 20 Hz to about 70 Hz, about 25 Hz to about 40 Hz, about 25 Hz to about 50 Hz, about 25 Hz to about 60 Hz, about 25 Hz to about 70 Hz, about 40 Hz to about 50 Hz, about 40 Hz to about 60 Hz, about 40 Hz to about 70 Hz, about 50 Hz to about 60 Hz, about 50 Hz to about 70 Hz, or about 60 Hz to about 70 Hz. In some embodiments, the filter may be configured to filter the frequency band of about 1 Hz, about 5 Hz, about 10 Hz, about 15 Hz, about 20 Hz, about 25 Hz, about 40 Hz, about 50 Hz, about 60 Hz, or about 70 Hz. In some embodiments, the filter may be configured to filter the frequency band of at least about 1 Hz, about 5 Hz, about 10 Hz, about 15 Hz, about 20 Hz, about 25 Hz, about 40 Hz, about 50 Hz, or about 60 Hz. In some embodiments, the filter may be configured to filter the frequency band of at most about 5 Hz, about 10 Hz, about 15 Hz, about 20 Hz, about 25 Hz, about 40 Hz, about 50 Hz, about 60 Hz, or about 70 Hz.

After passing the signal through the filter, the systems and methods described herein, may rectify 1006 the filtered signal. In the process of rectifying the signal, as understood by one of ordinary skill in the art, the signal will convert the alternating current detected signal to a direct current signal. The rectified signal may be additionally filtered with a low pass filter 1007 that may smooth the rectified signal. The signal may be subjected to a threshold detector 1008, where the threshold detector determines the onset of an episode of pain from a threshold intensity value of the rectified and smooth processed signals 1000 of EMG, ENG, accelerometer, gyroscope, magnetometer, pressure sensor signals, or any combination thereof. If an episode of pain is determined by the threshold detector 1008, the system may enable the delivery of electrical stimulation 1010, as described elsewhere herein.

In some embodiments, training may be supervised training. In some embodiments, training may be unsupervised training. In some embodiments, the data set may be a retrospective data set. In some embodiments, the data set may be prospectively developed dataset, and the machine learning model may be iteratively improved over time.

In some embodiments, the disclosure provided herein comprises a method to train a machine learning model with a data set that comprises sensed signal profiles and excitation signals that have and have not prevented episodes of pain. The method comprises the steps of: preprocessing, training, and predicting.

The method may extract training data from a database, or intake new data, described elsewhere herein. The preprocessing step may apply one or more transformations to standardize the training data or new data for the training step or the prediction step. The preprocessed training data may be passed to the training step, which may construct a machine learning model based on training data. The training step may further comprise a validation step, configured to validate the trained machine learning model using any appropriate validation algorithm (e.g., Stratified K-fold cross-validation). In some embodiments, the k-fold cross-validation comprises at least 1-fold, 2, folds, 3 folds, 4 folds, 5 folds, 6 folds, 7 folds, 8 folds, 9 folds, or 10 folds. In some embodiments, the k-fold cross-validation comprises up to 1-fold, 2 folds, 3 folds, 4 folds, 5 folds, 6 folds, 7 folds, 8 folds, 9 folds, or 10 folds.

The preprocessing step may apply one or more transformations to the training data to clean and normalize the data. The preprocessing step may be configured to discard parameters which contain spurious data or contain very few observations. The preprocessing module can be further configured to standardize the encoding of parameter values. The preprocessing step may recognize the encoding variation for the same value and standardize the dataset to have a uniform encoding for a given parameter value. The processing step may thus reduce irregularities in the input data for the training and prediction steps, thereby improving the robustness of the training and prediction steps.

The training step may utilize a machine learning algorithm or other algorithm to construct and train a machine learning model to be used in the association of an excitation stimulation, sensed signal profile, and the presence or lack thereof an episode of pain. A machine learning model may be constructed to capture, based on the training data, the statistical relationship, if any, between excitation stimulation parameters, sensed signal profiles, and the presence or lack thereof an episode of pain.

The machine learning algorithm may have an accuracy greater than about 60%, 70%, 80%, 85%, 90%, 95%, or 99%. The machine learning algorithm may have a positive predictive value greater than about 60%, 70%, 75%, 80%, 85%, 90%, 95%, or 99%. The machine learning algorithm may have a negative predictive value greater than about 60%, 70%, 80%, 90%, 95%, or 99%.

Machine learning data analysis, machine learning model training, or any combination thereof may be performed using one or more of many programming languages and platforms known in the art, such as R, Weka, Python, and/or MATLAB, for example.

The use of such closed-loop bioelectronic systems may potentially provide improved electrical stimulation devices to prevent or reduce pain of the individual. In some embodiments, the use of such closed-loop bioelectronic systems may provide a more precise approach to prevent or reduce or treat pain of the individual. In some embodiments, individuals having the implanted device may provide feedback to the parameters provided positive outcomes, negative outcomes, or neutral outcomes. In some embodiments, positive outcomes comprise preventing an episode of pain. In some embodiments, negative outcomes comprise not preventing an episode of pain, producing pain, or any combination thereof. In some embodiments, neutral outcomes comprise not preventing an episode of pain, not producing pain, or any combination thereof. In some embodiments, the positive outcomes, negative outcomes, neutral outcomes, sensor data, or any combination thereof from a plurality of individuals having the implanted device may be used in tuning algorithms to suggest changes to sensor thresholds and electrical stimulation patterns 124.

In some cases, the feedback and/or pain outcomes of the individual may be provided by a score or outcome of a survey, test, and/or questionnaire. The survey, test, and/or questionnaire may comprise the numeric pain rating scale (NPRS), patient global impression of change (PGIC), Cleveland Clinic Constipation and Incontinence Scores (CCCS and CCIS), International Consultation on Incontinence Questionnaire-Urinary Incontinence Short Form (ICIQ-SF-UI), Patient Global Impression of Improvement Scale (PGI-I), Female Sexual Function Index (FSFI), McGill Pain Questionnaire (MPQ), Visual Analogue Scale (VAS), or any combination thereof. In some cases, the feedback and/or pain outcomes of the individual may be measured. In some embodiments, the pain outcomes may be measured an increase in heart rate, and/or blood pressure, that track the individual's pain.

In some cases, the NPRS comprises a scale of 0 to 10, where a score of 0 indicates no pain and score of 10indicates extreme pain and/or the worst pain possible reported by the individual. In some embodiments, the ICIQ-SF-UI questionnaire provides one or more questions to determine frequency, severity and impact on quality of urinary incontinence of the individual. In some embodiments, the PGI-I provides a seven-point scale of objective improvement to a urinary tract condition of the individual from a time point before receiving treatment to a time after receiving treatment. The ICIQ-SF-UI questionnaire may comprise a four-item questionnaire including three scored items and one unscored self-diagnostic item. In some cases, the scored items of the ICIQ-SF-UI questionnaire are assigned values of 0 for no symptoms, 1 for slight symptoms, 2 for moderate symptoms, or 3 for frequent symptoms. In some cases, the average score of the items for the scored items of the ICIQ-SF-UI questionnaire is calculated and multiplied by 33.33 arriving at scores from a range of 0 to 100, where a score of 0 is no urinary incontinence and 100 is severe urinary incontinence. The PGI-I scale may comprise a value of 1 corresponding to substantial improvement to the individual's urinary tract condition and a value of 7 corresponding to substantially worsening of the individual's urinary tract condition. In some embodiments, the CCIS may comprise a score from 0 to 20, where 0 indicates perfect continence and 20 is complete incontinence. In some cases, the CCCS may comprise a score from 0 to 30, where 0 indicates no constipation and 30 indicates severe constipation. In some embodiments, the FSFI comprises a 19-time survey that measures a woman's sexual function in one or more domains. The one or more domains of the FSFI may comprise desire, arousal, lubrication, orgasm, satisfaction, pain, or any combination thereof. In some embodiments, the FSFI survey comprises a score that indicates a sum of the one or more domains, where the score may comprise a maximum of 36. In some cases, a score of up to about 26 indicates female sexual dysfunction. In some cases, the MPQ comprises 78 descriptors that evaluate our domains of an individual: sensory, affective, evaluative, and miscellaneous domains of an individual. Each descriptors associated with a given domain are weighted by the intensity of the descriptor on a scale of 0 as not intense, 1 mildly intense, 2 moderately intense, or 3 severe that are added together based on the individual's select of the descriptors for the various domains.

In some cases, the VAS measures and/or assesses an individual's pain and/or pain intensity. In some embodiments, the VAS comprises a test where the test comprises a graphic display of a line (e.g., on a screen or paper) with one end point representing a value of 0 indicating no pain and another end point representing a value of 10 indicating pain as bad as it could be. In some embodiments, an individual is asked to rate their pain on the VAS by drawing or indicating a line on the graphic display of the line where the individual's pain level is at the time of the assessment.

### Electrical Stimulation Pattern

In some embodiments, the electric stimulation pattern provided by an implantable pulse generator and one or more stimulator electrodes may be modified or changed to suit the needs of the individual in need thereof preventing an episode of pain. In some embodiments, the one or more stimulator electrodes 122 may output an electric stimulation pattern 124 in response to what is detected by the one or more sensors 120. In some embodiments, the electric stimulation pattern 124 comprises one or more electrical signals. For example, the electric stimulation pattern 124 comprises a continuous wave signal (e.g., an electric stimulation signal with a constant frequency in time) and a burst or beating signal superimposed onto the continuous wave signal. In some embodiments, the burst or beating signal may only be enabled for a short duration of time compared to the continual temporal aspect of the continuous wave signal. In some embodiments, the combination of the continuous wave signal and/or a burst or beating signal may increase a pain threshold of a subject allowing the stimulator to provide higher amplitude electric stimulation burst pattern to prevent episode of pain. In some embodiments, the frequency of the electric stimulation pattern may be modified or changed. The frequency pattern comprises a constant profile, swept profile, beating profile, burst profile, chirped profile, monophasic profile, biphasic profile, or any combination thereof. In some embodiments, the constant profile is comprised of excitation values at a constant amplitude with a frequency value of 0 Hz. In some embodiments, a swept profile comprises a signal with time varying frequency of excitation. In some embodiments, a beating profile comprises any combination of one or more excitation signals of varying frequency. In some embodiments, the burst profile comprises a signal with a constant frequency that is enveloped by a square, delta, sine, or any combination thereof envelope functions. In some embodiments, a monophasic profile comprises an excitation signal with only positive or negative amplitude (e.g., signal with values from 0 to −5V or 0 to 5V only) with a constant frequency. In some embodiments, the beating or burst profile comprises an electric stimulation pattern that is provided to a patient and/or subject for during an on-state for a first period of time and is not provided to a patient and/or subject during an off-state for a second period of time. In some embodiments, beating or burst profiles may provide an excitation signal that may provide for a lengthier period of muscle excitation without suffering muscle fatigue.

In some embodiments, the on-state and/or off-state comprises about 0.1 second(s) to about 6.5 s. In some embodiments, the on-state and/or off-state comprises about 0.1 s to about 0.5 s, about 0.1 s to about 1 s, about 0.1 s to about 1.2 s, about 0.1 s to about 1.5 s, about 0.1 s to about 2 s, about 0.1 s to about 2.5 s, about 0.1 s to about 3 s, about 0.1 s to about 3.5 s, about 0.1 s to about 4 s, about 0.1 s to about 5 s, about 0.1 s to about 6.5 s, about 0.5 s to about 1 s, about 0.5 s to about 1.2 s, about 0.5 s to about 1.5 s, about 0.5 s to about 2 s, about 0.5 s to about 2.5 s, about 0.5 s to about 3 s, about 0.5 s to about 3.5 s, about 0.5 s to about 4 s, about 0.5 s to about 5 s, about 0.5 s to about 6.5 s, about 1 s to about 1.2 s, about 1 s to about 1.5 s, about 1 s to about 2 s, about 1 s to about 2.5 s, about 1 s to about 3 s, about 1 s to about 3.5 s, about 1 s to about 4 s, about 1 s to about 5 s, about 1 s to about 6.5 s, about 1.2 s to about 1.5 s, about 1.2 s to about 2 s, about 1.2 s to about 2.5 s, about 1.2 s to about 3 s, about 1.2 s to about 3.5 s, about 1.2 s to about 4 s, about 1.2 s to about 5 s, about 1.2 s to about 6.5 s, about 1.5 s to about 2 s, about 1.5 s to about 2.5 s, about 1.5 s to about 3 s, about 1.5 s to about 3.5 s, about 1.5 s to about 4 s, about 1.5 s to about 5 s, about 1.5 s to about 6.5 s, about 2 s to about 2.5 s, about 2 s to about 3 s, about 2 s to about 3.5 s, about 2 s to about 4 s, about 2 s to about 5 s, about 2 s to about 6.5 s, about 2.5 s to about 3 s, about 2.5 s to about 3.5 s, about 2.5 s to about 4 s, about 2.5 s to about 5 s, about 2.5 s to about 6.5 s, about 3 s to about 3.5 s, about 3 s to about 4 s, about 3 s to about 5 s, about 3 s to about 6.5 s, about 3.5 s to about 4 s, about 3.5 s to about 5 s, about 3.5 s to about 6.5 s, about 4 s to about 5 s, about 4 s to about 6.5 s, or about 5 s to about 6.5 s. In some embodiments, the on-state and/or off-state comprises about 0.1 s, about 0.5 s, about 1 s, about 1.2 s, about 1.5 s, about 2 s, about 2.5 s, about 3 s, about 3.5 s, about 4 s, about 5 s, or about 6.5 s. In some embodiments, the on-state and/or of-state comprises at least about 0.1 s, about 0.5 s, about 1 s, about 1.2 s, about 1.5 s, about 2 s, about 2.5 s, about 3 s, about 3.5 s, about 4 s, or about 5 s. In some embodiments, the on-state and/or off-state comprises at most about 0.5 s, about 1 s, about 1.2 s, about 1.5 s, about 2 s, about 2.5 s, about 3 s, about 3.5 s, about 4 s, about 5 s, or about 6.5 s.

In some embodiments, an electrical stimulation pattern provided during an on-state comprises an oscillating electric stimulation pattern. In some embodiments, the oscillating electric stimulation pattern comprises one or more frequencies. In some embodiments, the frequency of the oscillating electrical stimulation may be chosen based upon prior knowledge of how similar subjects respond with a particular frequency or range of frequencies of the oscillating electrical stimulation pattern. In some embodiments, a low frequency (e.g., 2-15 Hz) may induces reductions in muscle contractility and thus prevent or reducing pain of the individual. In some embodiments, a higher frequency (e.g., 20 Hz or higher) may lead to potentiation of muscle contractions. In some embodiments, the frequency of the electrical stimulation pattern may be about 1 Hz to about 3000 Hz. In some embodiments, the frequency of the electrical stimulation pattern is about 1 Hz to about 5 Hz, about 1 Hz to about 10 Hz, about 1 Hz to about 50 Hz, about 1 Hz to about 100 Hz, about 1 Hz to about 500 Hz, about 1 Hz to about 1000 Hz, about 1 Hz to about 1500 Hz, about 1 Hz to about 2000 Hz, about 1 Hz to about 2500 Hz, about 1 Hz to about 3000 Hz, about 10 Hz to about 3000 Hz, about 50 Hz to about 2500 Hz, or about 100 Hz to about 2000 Hz. In some embodiments, the frequency of the electrical stimulation pattern is at least about 1 Hz, 10 Hz, 100 Hz, 500 Hz, 1000 Hz, 1500 Hz, or 2000 Hz. In some embodiments, the frequency of the electrical stimulation pattern is at most about 10 Hz, 100 Hz, 500 Hz, 1000 Hz, 1500 Hz, 2000 Hz, 2500 Hz, 3000 Hz, 4000 Hz, or 5000 Hz. In some embodiments, the frequency of the electrical stimulation pattern is about 1 Hz, 10 Hz, 100 Hz, 500 Hz, 1000 Hz, 1500 Hz, 2000 Hz, 2500 Hz, or 3000 Hz. In some embodiments, the frequency refers to mean frequency of the electrical stimulation pattern. In some embodiments, the frequency refers to the median frequency. In some embodiments, the frequency refers to the maximum frequency.

In some embodiments, a burst signal with an on-state and/or an off-state, described elsewhere herein may be provided to the subject to prevent fatigue on the one or more muscles innervated by the subject's pudendal nerve. In some embodiments, the burst electrical stimulation pattern comprises one or more frequencies described elsewhere herein.

In some embodiments, the amplitude of the electric stimulation pattern may be modified or changed. In some embodiments, the amplitude of the electrical stimulation pattern may be about 1 volt (V) to about 15 V. In some embodiments, the amplitude of the electrical stimulation pattern may be about 1 V to about 2 V, about 1 V to about 3 V, about 1 V to about 4 V, about 1 V to about 5 V, about 1 V to about 6 V, about 1 V to about 7 V, about 1 V to about 8 V, about 1 V to about 9 V, about 1 V to about 10 V, about 1 V to about 12 V, about 1 V to about 15 V, about 2 V to about 3 V, about 2 V to about 4 V, about 2 V to about 5 V, about 2 V to about 6 V, about 2 V to about 7 V, about 2 V to about 8 V, about 2 V to about 9 V, about 2 V to about 10 V, about 2 V to about 12 V, about 2 V to about 15 V, about 3 V to about 4 V, about 3 V to about 5 V, about 3 V to about 6 V, about 3 V to about 7 V, about 3 V to about 8 V, about 3 V to about 9 V, about 3 V to about 10 V, about 3 V to about 12 V, about 3 V to about 15 V, about 4 V to about 5 V, about 4 V to about 6 V, about 4 V to about 7 V, about 4 V to about 8 V, about 4 V to about 9 V, about 4 V to about 10 V, about 4 V to about 12 V, about 4 V to about 15 V, about 5 V to about 6 V, about 5 V to about 7 V, about 5 V to about 8 V, about 5 V to about 9 V, about 5 V to about 10 V, about 5 V to about 12 V, about 5 V to about 15 V, about 6 V to about 7 V, about 6 V to about 8 V, about 6 V to about 9 V, about 6 V to about 10 V, about 6 V to about 12 V, about 6 V to about 15 V, about 7 V to about 8 V, about 7 V to about 9 V, about 7 V to about 10 V, about 7 V to about 12 V, about 7 V to about 15 V, about 8 V to about 9 V, about 8 V to about 10 V, about 8 V to about 12 V, about 8 V to about 15 V, about 9 V to about 10 V, about 9 V to about 12 V, about 9 V to about 15 V, about 10 V to about 12 V, about 10 V to about 15 V, or about 12 V to about 15 V. In some embodiments, the amplitude of the electrical stimulation pattern may be about 1 V, about 2 V, about 3 V, about 4 V, about 5 V, about 6 V, about 7 V, about 8 V, about 9 V, about 10 V, about 12 V, or about 15 V. In some embodiments, the amplitude of the electrical stimulation pattern may be at least about 1 V, about 2 V, about 3 V, about 4 V, about 5 V, about 6 V, about 7 V, about 8 V, about 9 V, about 10 V, or about 12 V. In some embodiments, the amplitude of the electrical stimulation pattern may be at most about 2 V, about 3 V, about 4 V, about 5 V, about 6 V, about 7 V, about 8 V, about 9 V, about 10 V, about 12 V, or about 15 V. In some embodiments, the amplitude refers to the mean amplitude. In some embodiments, the amplitude refers to the median amplitude. In some embodiments, the amplitude refers to the maximum amplitude. In some embodiments, the amplitude refers to peak to peak amplitude.

In some embodiments, the amplitude of the electrical stimulation pattern may be about 0.05 milliampere (mA) to about 10 mA. In some embodiments, the amplitude of the electrical stimulation pattern may be about 0.05 mA to about 1 mA, about 0.05 mA to about 2 mA, about 0.05 mA to about 3 mA, about 0.05 mA to about 4 mA, about 0.05 mA to about 5 mA, about 0.05 mA to about 6 mA, about 0.05 mA to about 7 mA, about 0.05 mA to about 8 mA, about 0.05 mA to about 9 mA, about 0.05 mA to about 10 mA, about 1 mA to about 2 mA, about 1 mA to about 3 mA, about 1 mA to about 4 mA, about 1 mA to about 5 mA, about 1 mA to about 6 mA, about 1 mA to about 7 mA, about 1 mA to about 8 mA, about 1 mA to about 9 mA, about 1 mA to about 10 mA, about 2 mA to about 3 mA, about 2 mA to about 4 mA, about 2 mA to about 5 mA, about 2 mA to about 6 mA, about 2 mA to about 7 mA, about 2 mA to about 8 mA, about 2 mA to about 9 mA, about 2 mA to about 10 mA, about 3 mA to about 4 mA, about 3 mA to about 5 mA, about 3 mA to about 6 mA, about 3 mA to about 7 mA, about 3 mA to about 8 mA, about 3 mA to about 9 mA, about 3 mA to about 10 mA, about 4 mA to about 5 mA, about 4 mA to about 6 mA, about 4 mA to about 7 mA, about 4 mA to about 8 mA, about 4 mA to about 9 mA, about 4 mA to about 10 mA, about 5 mA to about 6 mA, about 5 mA to about 7 mA, about 5 mA to about 8 mA, about 5 mA to about 9 mA, about 5 mA to about 10 mA, about 6 mA to about 7 mA, about 6 mA to about 8 mA, about 6 mA to about 9 mA, about 6 mA to about 10 mA, about 7 mA to about 8 mA, about 7 mA to about 9 mA, about 7 mA to about 10 mA, about 8 mA to about 9 mA, about 8 mA to about 10 mA, or about 9 mA to about 10 mA. In some embodiments, the amplitude of the electrical stimulation pattern may be about 0.05 mA, about 1 mA, about 2 mA, about 3 mA, about 4 mA, about 5 mA, about 6 mA, about 7 mA, about 8 mA, about 9 mA, or about 10 mA. In some embodiments, the amplitude of the electrical stimulation pattern may be at least about 0.05 mA, about 1 mA, about 2 mA, about 3 mA, about 4 mA, about 5 mA, about 6 mA, about 7 mA, about 8 mA, or about 9 mA. In some embodiments, the amplitude of the electrical stimulation pattern may be at most about 1 mA, about 2 mA, about 3 mA, about 4 mA, about 5 mA, about 6 mA, about 7 mA, about 8 mA, about 9 mA, or about 10 mA. In some embodiments, the amplitude refers to the mean amplitude. In some embodiments, the amplitude refers to the median amplitude. In some embodiments, the amplitude refers to the maximum amplitude.

In some embodiments, the pulse width of the electric stimulation pattern may be modified or changed. In some embodiments, the pulse width of the electrical stimulation pattern may be about 60 μs to about 390 μs. In some embodiments, the pulse width of the electrical stimulation pattern may be about 60 μs to about 90 μs, about 60 μs to about 120 μs, about 60 μs to about 150 μs, about 60 μs to about 180 μs, about 60 μs to about 210 μs, about 60 μs to about 240 μs, about 60 μs to about 270 us, about 60 μs to about 300 μs, about 60 μs to about 330 μs, about 60 μs to about 360 μs, about 60 μs to about 390 μs, about 90 us to about 120 μs, about 90 μs to about 150 μs, about 90 μs to about 180 μs, about 90 μs to about 210 μs, about 90 μs to about 240 μs, about 90 μs to about 270 μs, about 90 μs to about 300 μs, about 90 μs to about 330 μs, about 90 μs to about 360 μs, about 90 μs to about 390 μs, about 120 μs to about 150 μs, about 120 μs to about 180 μs, about 120 μs to about 210 μs, about 120 μs to about 240 μs, about 120 μs to about 270 μs, about 120 μs to about 300 μs, about 120 μs to about 330 μs, about 120 μs to about 360 μs, about 120 μs to about 390 μs, about 150 μs to about 180 μs, about 150 μs to about 210 μs, about 150 μs to about 240 μs, about 150 μs to about 270 μs, about 150 μs to about 300 μs, about 150 μs to about 330 μs, about 150 μs to about 360 μs, about 150 μs to about 390 μs, about 180 μs to about 210 μs, about 180 μs to about 240 μs, about 180 μs to about 270 μs, about 180 μs to about 300 μs, about 180 μs to about 330 μs, about 180 μs to about 360 μs, about 180 μs to about 390 μs, about 210 μs to about 240 μs, about 210 μs to about 270 μs, about 210 μs to about 300 μs, about 210 μs to about 330 μs, about 210 μs to about 360 μs, about 210 μs to about 390 μs, about 240 μs to about 270 μs, about 240 μs to about 300 μs, about 240 μs to about 330 μs, about 240 μs to about 360 μs, about 240 μs to about 390 μs, about 270 μs to about 300 μs, about 270 μs to about 330 μs, about 270 μs to about 360 μs, about 270 μs to about 390 μs, about 300 μs to about 330 μs, about 300 μs to about 360 μs, about 300 μs to about 390 μs, about 330 μs to about 360 μs, about 330 μs to about 390 μs, or about 360 μs to about 390 μs. In some embodiments, the pulse width of the electrical stimulation pattern may be about 60 μs, about 90 μs, about 120 μs, about 150 μs, about 180 μs, about 210 μs, about 240 μs, about 270 μs, about 300 μs, about 330 μs, about 360 μs, or about 390 μs. In some embodiments, the pulse width of the electrical stimulation pattern may be at least about 60 μs, about 90 μs, about 120 μs, about 150 μs, about 180 μs, about 210 μs, about 240 μs, about 270 μs, about 300 μs, about 330 μs, or about 360 μs. In some embodiments, the pulse width of the electrical stimulation pattern may be at most about 90 μs, about 120 μs, about 150 μs, about 180 μs, about 210 μs, about 240 μs, about 270 μs, about 300 μs, about 330 μs, about 360 μs, or about 390 μs. In some embodiments, the pulse width refers to the mean pulse width. In some embodiments, the pulse width refers to the median pulse width. In some embodiments, the pulse width refers to the maximum pulse width.

### Device

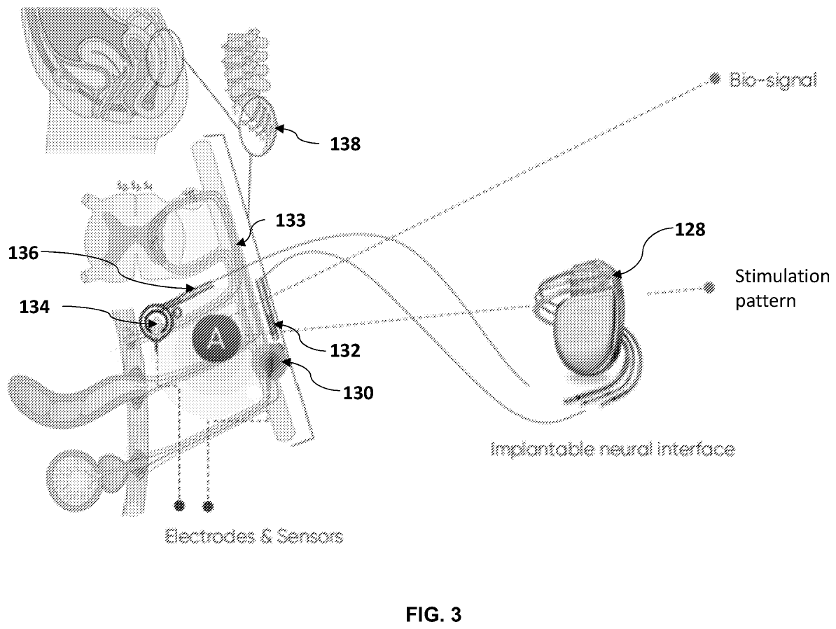

FIG. 3 shows an exemplary embodiment of the devices and methods described herein. In some embodiments, the implanted device targets a peripheral nerve 133 by implanting sensors 130 and 134 configured to sense a parameter associate with an episode of pain of the individual and stimulation electrodes 132 and 136 adjacent to or at the peripheral nerve. In some cases, the peripheral nerve may be a pudendal nerve. One or more of the sensors 130 and 134 may detect a parameter associated with an episode of pain. In some embodiments, the parameter is classified and, the stimulator electrodes 130 and 132 deliver an adapted electrical stimulation to the nerve, wherein the adapted electrical stimulation is adapted from detected parameter, and may further augment the basal electrical stimulation, if provided, to reduce pain of the individual.

In some embodiments, providing electrical stimulation to the pudendal nerve instead of the sacral nerve may provide a greater precision as the pudendal nerve, or branches thereof, is inferior to the sacral nerve and may be closer to the organs and tissues associated with chronic pelvic pain (CPP) or pelvic pain than the sacral nerves. The sensors capture the signal to classify any parameters associated with an episode of pain, and the stimulator electrodes of the device deliver the electrical stimulation (stimulation pattern), which is adapted from the detected parameter to account for the episode of pain. In some embodiments, the adapted electrical stimulation constructively interferes or adds to the individual's electrical signal associated with pain at or near the target site in order to reduce pain. In some embodiments, the adapted electrical stimulation destructively interferes with the individual's electrical signal associated with pain at or near the target site in order to reduce pain.

### Sensors

In some embodiments, the sensor is configured to sense a parameter associated with an episode of pain or that indicates the individual may have an episode of pain. In some embodiments, the sensor may be configured to sense a contraction of a muscle of the individual. In some embodiments, the sensor is configured to sense bulk motion or anatomical stress of an individual. In some embodiments, the sensor is configured to detect electromyography (EMG) signals. In some embodiments, the sensor is configured to sense myoelectric activity. In some embodiments, an EMG signal threshold indicates that a contraction of at least one pelvic muscle has occurred. In some embodiments, the strength of the EMG signal is proportional to the strength of the contraction of at least one pelvic muscle. In some embodiments, the sensor detects an action potential signal. In some embodiments, the device comprises an amplifier to amplify the signal obtained by the sensor, thereby facilitating analysis and classification of the signal by the processor.

In some embodiments, the parameter associated with the episode of pain of the individual comprises an electrical parameter, a pressure parameter, a motion parameter, or any combination thereof. In some embodiments, the parameter associated with the episode of pain comprises a physiological signal, EMG signal, ENG signal, digital signal, patient actuated signal based on perception of pain or increased pain, pressure signal, muscle contraction event signal, motion signal, orientation signal, posture signal, GPS signal, speed signal, location signal, time of day signal, time interval signal, or any combination thereof. In some embodiments, the sensor is configured to detect the parameter.

In some embodiments, the sensor comprises a sensor electrode, receiver, pressure sensor, accelerometer, gyroscope, magnetometer, global positioning system, or any combination thereof. In some embodiments, the parameter detected by the receiver, pressure sensor, accelerometer, gyroscope, magnetometer, global positioning system, or any combination thereof is associated with a pressure, muscle contraction event, motion, orientation, posture, GPS reading, speed, location, time of day, time interval, or any combination thereof. In some embodiments, the parameter detected by the sensor electrode, receiver, pressure sensor, or any combination thereof is associated with a physiological event, EMG signal, ENG signal, digital signal, patient actuated input based on perception of pain or increased pain, pressure, muscle contraction event, or any combination thereof. In some embodiments, the receiver comprises a controller receiver, digital receiver, radio receiver, or any combination thereof. In some embodiments, the sensor is configured to detect activity of a muscle of the individual, and wherein the activity is associated with an EMG signal, ENG signal, digital signal, patient actuated signal based on perception of pain or increased pain, pressure signal, muscle contraction event signal, or any combination thereof. In some embodiments, the muscle comprises a pelvic floor muscle. In some embodiments, the parameter is associated with at least one of contraction or increased tone of the pelvic floor muscle.

In some embodiments, the sensor is configured to detect activity of a sensed nerve of the individual. In some embodiments, the activity of the sensed nerve is associated with an ENG signal of the sensed nerve. In some embodiments, the sensed nerve comprises a motor nerve fiber, a larger diameter afferent nerve, or any combination, a pudendal nerve, a sacral nerve, a nerve of the pelvic plexus, or any combination thereof.

In some embodiments, the sensor is implanted within the pelvis or pelvic region of the individual. In some embodiments, the sensor is implanted at or adjacent to a pudendal nerve. In some embodiments, the sensor is implanted at or adjacent to a sacral nerve. In some embodiments, the sensor is implanted at or adjacent to a nerve of the pelvic plexus. In some embodiments, the sensor is implanted within or adjacent to one or more of the pelvic muscles. In some embodiments, the sensor is implanted at or adjacent to the pelvic floor. In some embodiments, the devices described herein comprises a plurality of sensors. In some embodiments, the devices described herein comprises one or more sensors. In some embodiments, the devices described herein comprises a different sensor, or a second sensor. In some embodiments, the sensor may detect the signal from a muscle area innervated by a first pudendal nerve and the different or second sensor may detect signal from a muscle area innervated by a second pudendal nerve. In some embodiments, the sensor may detect a signal from a muscle area innervated by a first sacral nerve and the different or second sensor detects a signal from a muscle area innervated by a second sacral nerve.

In some embodiments, the sensor comprises a casing and a lead. In some embodiments, the casing is made of titanium, titanium alloy, tantalum, or any combination thereof. In some embodiments, the lead is made of a metal alloy. In some embodiments, a sensor and a stimulator electrode are attached to a single lead. In some embodiments, the lead is electrically coupled to one or more sensors or one or more stimulator electrodes. In some embodiments, a sensor and a stimulator electrode are attached to separate leads. In some embodiments, the one or more sensors comprises bioelectrical sensors.

### Electrodes

In some embodiments, the stimulator electrode comprises a first stimulator. In some embodiments, the first stimulator is implanted at or adjacent to a first anatomical site. In some embodiments, the first anatomical site is in the pelvic area. In some embodiments, the first anatomical site is adjacent to or at a nerve, as described elsewhere herein. In some embodiments, the first anatomical site is adjacent to or at a pudendal nerve. In some embodiments, the first anatomical site is adjacent to or at a sacral nerve. In some embodiments, the first anatomical site is adjacent to or at a pelvic plexus nerve. In some embodiments, the first anatomical site is at or adjacent to a muscle cell, muscle fiber, muscle tissue, muscle, or any combination thereof. In some embodiments, the first anatomical site is at or adjacent to a pelvic muscle. In some embodiments, the first anatomical site is at or adjacent to a pelvic floor muscle.

In some embodiments, the stimulator electrode further comprises a second stimulator. In some embodiments, the second stimulator is implanted at or adjacent to a second anatomical site. In some embodiments, the second anatomical site is in the pelvic area. In some embodiments, the second anatomical site is adjacent to or at a nerve. In some embodiments, the second anatomical site is adjacent to or at a pudendal nerve. In some embodiments, the second anatomical site is adjacent to or at a sacral nerve. In some embodiments, the second anatomical site is at or adjacent to a muscle, wherein the muscle comprises a muscle cell, muscle fiber, muscle tissue, or any combination thereof. In some embodiments, the second anatomical site is at or adjacent to a pelvic muscle. In some embodiments, the second anatomical site is at or adjacent to a pelvic floor muscle.

In some embodiments, the first anatomical site is adjacent to or at a first pudendal nerve. In some embodiments, the second anatomical site is adjacent to or at the first pudendal nerve or a second pudendal nerve. In some embodiments, the first anatomical site is adjacent to or at a first sacral nerve. In some embodiments, the second anatomical site is adjacent to or at the first sacral nerve or a second sacral nerve. In some embodiments, the first anatomical site is adjacent to or at a pudendal nerve and the second anatomical site is adjacent to or at the pudendal nerve, a sacral nerve, a muscle, or any combination thereof. In some embodiments, the first anatomical site is adjacent to or at a sacral nerve and the second anatomical site is adjacent to or at the sacral nerve, a pudendal nerve, a muscle, or any combination thereof.

In some embodiments, the first stimulator electrically stimulates a nerve or a muscle and the second stimulator electrically stimulates a nerve or a muscle. In some embodiments, the nerve comprises a motor nerve fiber, a larger diameter afferent nerve, or any combination thereof. In some embodiments, the nerve gates peripheral nociception, spinal activity, or any combination thereof. In some embodiments, the nerve stimulates muscle contraction, modulates muscle activity, or any combination thereof. In some embodiments, the muscle comprises a pelvic muscle. In some embodiments, the muscle comprises a pelvic floor muscle.

In some embodiments, a base electrical stimulation is provided by the stimulator electrode before the adapted electrical stimulation is provided. In some embodiments, the adapted electrical stimulation is different from the base electrical stimulation. In some embodiments, the adapted electrical stimulation boosts the base electrical stimulation. In some embodiments, boosts comprise increasing the stimulation amplitude, frequency, intensity, or any combination thereof. In some embodiments, the adapted electrical situation, alone or together with the base electrical stimulation, reduces the pain of the episode. In some embodiments, the adapted electrical stimulation is configured to reduce pain of the episode when provided alone or together with the base electrical stimulation.

In some embodiments, the adapted electrical stimulation comprises one or more stimulation patterns. In some embodiments, the adapted electrical stimulation comprises a first stimulation pattern. In some embodiments, the first stimulation pattern is provided by the first stimulator. In some embodiments, the adapted electrical stimulation comprises a second stimulation pattern. In some embodiments, the second stimulation pattern is provided by the first stimulator, the second stimulator, or any combination thereof. In some embodiments, the first stimulation pattern is based on a first parameter. In some embodiments, the second stimulation pattern is based on the first parameter, a second parameter, or any combination thereof. In some embodiments, the one or more stimulation patterns are configured to reduce pain of the episode based on at least one parameter associated with the episode of pain.

In some embodiments, the one or more stimulation patterns comprise excitation values for intensity, frequency, phase, pulse width, or any combination thereof. In some embodiments, the first and second stimulation patters are the same. In some embodiments, the first and second stimulation patters differ in at least one of intensity, frequency, phase, or pulse width.

In some embodiments, software is used to generate the one or more stimulation patterns based on at least a first parameter. In some embodiments, software is used to generate a first stimulation pattern based on a first parameter and a second stimulation pattern based on at least one of the first parameter or a second parameter. In some embodiments, the adapted electrical stimulation is further sustained, modified, redirected, withheld, or cancelled based on a second parameter and wherein the second parameter is later in time to the first parameter.

In some embodiments, the sensor and the stimulator electrode are electrically coupled to a processor. In some embodiments, a parameter is provided by the individual via a controller in wireless communication with the processor. In some embodiments, the sensor is configured to transmit data to the processor. In some embodiments, the data is associated with the parameter that is detected by the sensor. In some embodiments, the sensor and the stimulator electrode may be operatively coupled to a processor and a non-transitory computer readable medium that includes software. In some embodiments, the sensor may be calibrated by the individual using an external input device that interfaces with the software. In some embodiments, the software may be configured to record a signal from the sensor. In some embodiments, the software may be configured to adjust the sensor in response to the signal.

In some embodiments, the sensor and the stimulator electrode may be located on a single lead. In some embodiments, one or more sensors and one or more stimulator electrodes may be located on a single lead. In some embodiments, one sensor and one stimulator electrode may be located on a single lead. In some embodiments, the sensor and the stimulator electrode may be located on separate leads. In some embodiments, the sensor and the stimulator electrode may each be located on its own lead. In some embodiments, the one or more sensors and one or more stimulator electrodes may be in a linear geometry, triangular geometry, square geometry, hexagonal geometry, or a general polygonal geometry. In some embodiments, the electrodes located on a single lead may be spaced by a distance. In some embodiments, the spacing provides the capability to stimulate multiple locations along the length of the nerve. In some embodiments, the electrodes may be separated by a distance of at least about 1 mm, about 1.5 mm, about 2 mm, about 2.5 mm, about 5 mm, about 10 mm, about 20 mm, about 30 mm, about 40 mm, about 50 mm, or about 60 mm. In some embodiments, the electrodes may be separated by a distance of at most about 1.5 mm, about 2 mm, about 2.5 mm, about 5 mm, about 10 mm, about 20 mm, about 30 mm, about 40 mm, about 50 mm, about 60 mm, or about 80 mm.

In some embodiments, the device comprises one or more leads. In some embodiments, the device comprises at least two leads. In some embodiments, the device comprises at least 1, 2, 3, 4, 5, 6, 7, 8, 9, or 10 leads. In some embodiments, the device comprises at most 1, 2, 3, 4, 5, 6, 7, 8, 9, or 10 leads. In some embodiments, the device comprises 1, 2, 3, 4, 5, 6, 7, 8, 9, or 10 leads. In some embodiments, each lead comprises one or more electrodes. In some embodiments, the one or more electrodes comprises one or more sensor and/or stimulation electrodes. In some embodiments, the one or more electrodes on a lead comprises about 1 electrode to about 10 electrodes. In some embodiments, the one or more electrodes on a lead comprises about 1 electrode to about 2 electrodes, about 1 electrode to about 3 electrodes, about 1 electrode to about 4 electrodes, about 1 electrode to about 5 electrodes, about 1 electrode to about 6 electrodes, about 1 electrode to about 7 electrodes, about 1 electrode to about 8 electrodes, about 1 electrode to about 9 electrodes, about 1 electrode to about 10 electrodes, about 2 electrodes to about 3 electrodes, about 2 electrodes to about 4 electrodes, about 2 electrodes to about 5 electrodes, about 2 electrodes to about 6 electrodes, about 2 electrodes to about 7 electrodes, about 2 electrodes to about 8 electrodes, about 2 electrodes to about 9 electrodes, about 2 electrodes to about 10 electrodes, about 3 electrodes to about 4 electrodes, about 3 electrodes to about 5 electrodes, about 3 electrodes to about 6 electrodes, about 3 electrodes to about 7 electrodes, about 3 electrodes to about 8 electrodes, about 3 electrodes to about 9 electrodes, about 3 electrodes to about 10 electrodes, about 4 electrodes to about 5 electrodes, about 4 electrodes to about 6 electrodes, about 4 electrodes to about 7 electrodes, about 4 electrodes to about 8 electrodes, about 4 electrodes to about 9 electrodes, about 4 electrodes to about 10 electrodes, about 5 electrodes to about 6 electrodes, about 5 electrodes to about 7 electrodes, about 5 electrodes to about 8 electrodes, about 5 electrodes to about 9 electrodes, about 5 electrodes to about 10 electrodes, about 6 electrodes to about 7 electrodes, about 6 electrodes to about 8 electrodes, about 6 electrodes to about 9 electrodes, about 6 electrodes to about 10 electrodes, about 7 electrodes to about 8 electrodes, about 7 electrodes to about 9 electrodes, about 7 electrodes to about 10 electrodes, about 8 electrodes to about 9 electrodes, about 8 electrodes to about 10 electrodes, or about 9 electrodes to about 10 electrodes. In some embodiments, the one or more electrodes on a lead comprises about 1 electrode, about 2 electrodes, about 3 electrodes, about 4 electrodes, about 5 electrodes, about 6 electrodes, about 7 electrodes, about 8 electrodes, about 9 electrodes, or about 10 electrodes. In some embodiments, the one or more electrodes on a lead comprises at least about 1 electrode, about 2 electrodes, about 3 electrodes, about 4 electrodes, about 5 electrodes, about 6 electrodes, about 7 electrodes, about 8 electrodes, or about 9 electrodes. In some embodiments, the one or more electrodes on a lead comprises at most about 2 electrodes, about 3 electrodes, about 4 electrodes, about 5 electrodes, about 6 electrodes, about 7 electrodes, about 8 electrodes, about 9 electrodes, or about 10 electrodes.

In some embodiments, each electrode comprises a length, whereby the length may provide localized excitation of one or more nerves of the sacral or pudendal nerve. In some embodiments, each electrode comprises a length of about 0.1 millimeter (mm) to about 2 mm. In some embodiments, each electrode comprises a length of about 0.1 mm to about 0.3 mm, about 0.1 mm to about 0.5 mm, about 0.1 mm to about 0.7 mm, about 0.1 mm to about 0.8 mm, about 0.1 mm to about 1 mm, about 0.1 mm to about 1.2 mm, about 0.1 mm to about 1.4 mm, about 0.1 mm to about 1.5 mm, about 0.1 mm to about 2 mm, about 0.3 mm to about 0.5 mm, about 0.3 mm to about 0.7 mm, about 0.3 mm to about 0.8 mm, about 0.3 mm to about 1 mm, about 0.3 mm to about 1.2 mm, about 0.3 mm to about 1.4 mm, about 0.3 mm to about 1.5 mm, about 0.3 mm to about 2 mm, about 0.5 mm to about 0.7 mm, about 0.5 mm to about 0.8 mm, about 0.5 mm to about 1 mm, about 0.5 mm to about 1.2 mm, about 0.5 mm to about 1.4 mm, about 0.5 mm to about 1.5 mm, about 0.5 mm to about 2 mm, about 0.7 mm to about 0.8 mm, about 0.7 mm to about 1 mm, about 0.7 mm to about 1.2 mm, about 0.7 mm to about 1.4 mm, about 0.7 mm to about 1.5 mm, about 0.7 mm to about 2 mm, about 0.8 mm to about 1 mm, about 0.8 mm to about 1.2 mm, about 0.8 mm to about 1.4 mm, about 0.8 mm to about 1.5 mm, about 0.8 mm to about 2 mm, about 1 mm to about 1.2 mm, about 1 mm to about 1.4 mm, about 1 mm to about 1.5 mm, about 1 mm to about 2 mm, about 1.2 mm to about 1.4 mm, about 1.2 mm to about 1.5 mm, about 1.2 mm to about 2 mm, about 1.4 mm to about 1.5 mm, about 1.4 mm to about 2 mm, or about 1.5 mm to about 2 mm. In some embodiments, each electrode comprises a length of about 0.1 mm, about 0.3 mm, about 0.5 mm, about 0.7 mm, about 0.8 mm, about 1 mm, about 1.2 mm, about 1.4 mm, about 1.5 mm, or about 2 mm. In some embodiments, each electrode comprises a length of at least about 0.1 mm, about 0.3 mm, about 0.5 mm, about 0.7 mm, about 0.8 mm, about 1 mm, about 1.2 mm, about 1.4 mm, or about 1.5 mm. In some embodiments, each electrode comprises a length of at most about 0.3 mm, about 0.5 mm, about 0.7 mm, about 0.8 mm, about 1 mm, about 1.2 mm, about 1.4 mm, about 1.5 mm, or about 2 mm.

In some embodiments, the one or more leads comprises a length between the proximal tip of the lead to the distal end electrically coupled to the stimulator. In some embodiments, the length of the lead may vary based upon the anatomy of the individual or subject receiving the implanted device and leads. In some embodiments, the lead length comprises a length, whereby the lead electrodes may be placed near and/or adjacent to the sacral, pudendal, and/or pelvic plexus nerve yet reach the placement of the stimulator in buttock fat pockets of the subject. In some embodiments, the one or more leads comprises a length of about 20 centimeters (cm) to about 50 cm. In some embodiments, the one or more leads comprises a length of about 20 cm to about 25 cm, about 20 cm to about 30 cm, about 20 cm to about 35 cm, about 20 cm to about 40 cm, about 20 cm to about 45 cm, about 20 cm to about 50 cm, about 25 cm to about 30 cm, about 25 cm to about 35 cm, about 25 cm to about 40 cm, about 25 cm to about 45 cm, about 25 cm to about 50 cm, about 30 cm to about 35 cm, about 30 cm to about 40 cm, about 30 cm to about 45 cm, about 30 cm to about 50 cm, about 35 cm to about 40 cm, about 35 cm to about 45 cm, about 35 cm to about 50 cm, about 40 cm to about 45 cm, about 40 cm to about 50 cm, or about 45 cm to about 50 cm. In some embodiments, the one or more leads comprises a length of about 20 cm, about 25 cm, about 30 cm, about 35 cm, about 40 cm, about 45 cm, or about 50 cm. In some embodiments, the one or more leads comprises a length of at least about 20 cm, about 25 cm, about 30 cm, about 35 cm, about 40 cm, or about 45 cm. In some embodiments, the one or more leads comprises a length of at most about 25 cm, about 30 cm, about 35 cm, about 40 cm, about 45 cm, or about 50 cm.

In some embodiments, the one or more leads comprises a diameter. In some embodiments, the diameter of the lead may vary based upon the anatomy of the individual or subject receiving the implanted device and leads. In some embodiments, the lead diameter comprises a diameter, whereby the diameter provides a form factor for minimally invasive placement of the lead in the subject. In some embodiments, the diameter of the lead comprises a diameter at which the lead will resist breakage. In some embodiments, the one or more leads may have an outer diameter of about 0.1 mm to about 2 mm. In some embodiments, the one or more leads may have an outer diameter of about 0.1 mm to about 0.2 mm, about 0.1 mm to about 0.3 mm, about 0.1 mm to about 0.5 mm, about 0.1 mm to about 0.8 mm, about 0.1 mm to about 1 mm, about 0.1 mm to about 1.2 mm, about 0.1 mm to about 1.4 mm, about 0.1 mm to about 1.5 mm, about 0.1 mm to about 2 mm, about 0.2 mm to about 0.3 mm, about 0.2 mm to about 0.5 mm, about 0.2 mm to about 0.8 mm, about 0.2 mm to about 1 mm, about 0.2 mm to about 1.2 mm, about 0.2 mm to about 1.4 mm, about 0.2 mm to about 1.5 mm, about 0.2 mm to about 2 mm, about 0.3 mm to about 0.5 mm, about 0.3 mm to about 0.8 mm, about 0.3 mm to about 1 mm, about 0.3 mm to about 1.2 mm, about 0.3 mm to about 1.4 mm, about 0.3 mm to about 1.5 mm, about 0.3 mm to about 2 mm, about 0.5 mm to about 0.8 mm, about 0.5 mm to about 1 mm, about 0.5 mm to about 1.2 mm, about 0.5 mm to about 1.4 mm, about 0.5 mm to about 1.5 mm, about 0.5 mm to about 2 mm, about 0.8 mm to about 1 mm, about 0.8 mm to about 1.2 mm, about 0.8 mm to about 1.4 mm, about 0.8 mm to about 1.5 mm, about 0.8 mm to about 2 mm, about 1 mm to about 1.2 mm, about 1 mm to about 1.4 mm, about 1 mm to about 1.5 mm, about 1 mm to about 2 mm, about 1.2 mm to about 1.4 mm, about 1.2 mm to about 1.5 mm, about 1.2 mm to about 2 mm, about 1.4 mm to about 1.5 mm, about 1.4 mm to about 2 mm, or about 1.5 mm to about 2 mm. In some embodiments, the one or more leads may have an outer diameter of about 0.1 mm, about 0.2 mm, about 0.3 mm, about 0.5 mm, about 0.8 mm, about 1 mm, about 1.2 mm, about 1.4 mm, about 1.5 mm, or about 2 mm. In some embodiments, the one or more leads may have an outer diameter of at least about 0.1 mm, about 0.2 mm, about 0.3 mm, about 0.5 mm, about 0.8 mm, about 1 mm, about 1.2 mm, about 1.4 mm, or about 1.5 mm. In some embodiments, the one or more leads may have an outer diameter of at most about 0.2 mm, about 0.3 mm, about 0.5 mm, about 0.8 mm, about 1 mm, about 1.2 mm, about 1.4 mm, about 1.5 mm, or about 2 mm.

In some embodiments, the one or more leads described elsewhere herein comprises an internal stylet and/or mandrel configured to provide rigidity to the lead for implantation and/or insertion to a subject. In some embodiments, the internal stylet may be removed from the lead once the lead has been inserted and implanted. In some embodiments, the one or more leads described elsewhere herein may be sterilizable with conventional methods of sterilization used in the medical field, e.g., gas sterilization, steam sterilization, UV sterilization, etc.

In some embodiments, the one or more leads, described elsewhere herein, may be electrically coupled to the electric stimulator, described elsewhere herein. In some embodiments, the one or more leads may be coupled to the stimulator and may at a later point in time be uncoupled from the stimulator. In some embodiments, the one or more leads may couple to the electric stimulator with a quick release electrical coupling. In some embodiments, the one or more leads may be coupled to the electric stimulator by a set screw fastener, whereby a lead is inserted into a hollow cylindrical geometry in electrical communication with the electric stimulator internal circuitry. The lead may then be fastened i.e., held in tension against the inner wall of the hollow cylindrical geometry, to the conductive hollow cylindrical geometry with a non-conductive machine set screw. The one or more leads may be placed into the electric stimulator prior to or during the surgical implantation procedure.

In some embodiments, the stimulator electrode may provide an electrical stimulation to the pudendal nerve. In some embodiments, the stimulator electrode may provide an electrical stimulation to the sacral nerve. In some embodiments, the stimulator electrode may provide an electrical stimulation to one or more of the nerves innervating the pelvic muscles. In some embodiments, the stimulator electrode may stimulate a muscle cell, muscle fiber, muscle tissue, or any combination thereof, wherein the muscle is a pelvic muscle.

In some embodiments, the electrode comprises one or more electrodes or leads (e.g., a first and second electrode)). In some embodiments, the electrode comprises one or more stimulator electrodes. In some embodiments, an electrode comprises a first stimulator electrode configured to stimulate one pudendal nerve, and a second stimulator electrode configured to stimulate another spatially independent region of the same pudendal nerve. In some embodiments, the first stimulator electrode may stimulate the main trunk of the pudendal nerve and the second stimulator electrode may stimulate the distal nerve of the pudendal nerve. In some embodiments, the distal nerve of the pudendal nerve comprises branches thereof the distal pudendal nerve. In some embodiments, the first stimulator electrode may stimulate the trunk of the pudendal nerve and the second stimulator electrode may stimulate a main branch of the pudendal nerve e.g., dorsal genital nerve. In some embodiments, the stimulator electrode may provide an electrical stimulation to one or more of the nerves innervating the pelvic floor muscles. In some embodiments, the devices described herein comprises a plurality of stimulator electrodes. In some embodiments, the devices described herein comprises one or more stimulator electrodes. In some embodiments, the devices described herein comprises a different stimulator electrode, or a second stimulator electrode. In some embodiments, the stimulator electrode may stimulate a first pudendal nerve and the different or second stimulator electrode stimulates a second pudendal nerve. In some embodiments, the stimulator electrode may stimulate a first sacral nerve and the different or second stimulator electrode stimulates a second sacral nerve.

In some embodiments, the stimulator electrode of the device may provide a base electrical stimulation. In some embodiments, the stimulator electrode of the device may provide a base electrical stimulation at a lower intensity level than the electrical stimulation provided to prevent an episode of pain. In some embodiments, base electrical stimulation comprises constant frequency, amplitude, current, or any combination thereof. In some embodiments, the intensity or duration of the electrical stimulation provided may prevent an episode of pain varies according to the individual's response to a possible episode of pain that is sensed by the sensor. In some embodiments, the individual's response to prevent a possible episode of pain that is sensed by the sensor may be insufficient on its own to prevent the episode of pain and the electrical stimulation delivered by the stimulator electrode of the device provides sufficient stimulation, together with the response, to prevent the episode of pain. In some embodiments, the individual's response to prevent a possible episode of pain that may be sensed by the sensor combined with the electrical stimulation delivered by the stimulator electrode of the device provides sufficient stimulation to trigger the action potential of the muscles responsible for pain, resulting in contraction of the muscle. In some embodiments, the combined stimulation from the individual and the device may result in contraction of a muscle. In some embodiments, the muscle comprises a urethral sphincter. In some embodiments, the muscle comprises an anal sphincter. In some embodiments, the muscle comprises one or more of the pelvic floor muscles.

In some embodiments, the sensor comprises casing and a lead. In some embodiments, the casing may be made of titanium or a titanium alloy. In some embodiments, the lead may be made of a metal alloy.

### Device Anchors

In some embodiments, the devices may be anchored when implanted by one the one or more surgical device. In some embodiments, anchoring of the devices may be achieved by sliding the device over the lead, described elsewhere herein, and then compressing it onto the lead using ligatures such that it is immobile. These ligatures may be used to fix the anchoring device to native adjacent tissue such as ligament or periosteum. In some embodiments, the device comprises groves for the purpose of aligning compression ligatures. In some embodiments, the anchoring device comprises a torque system to compress the device onto the lead such that it is immobile. In some embodiments, the device may be compressed at a single point onto the lead. In some embodiments, the device is compressed at two or more points onto the lead. The electric stimulator comprises radio-opaque markers that may permit visualization of the electric stimulator under x-ray e.g., fluoroscopy during and/or after implantation. In some embodiments, fluoroscopy may be used alone or in combination with EMG sensor readings of pelvic floor muscles to verify placement or to adjustment placement of sensor leads, stimulator electrode leads, and/or the stimulator.

### Electrical Signal

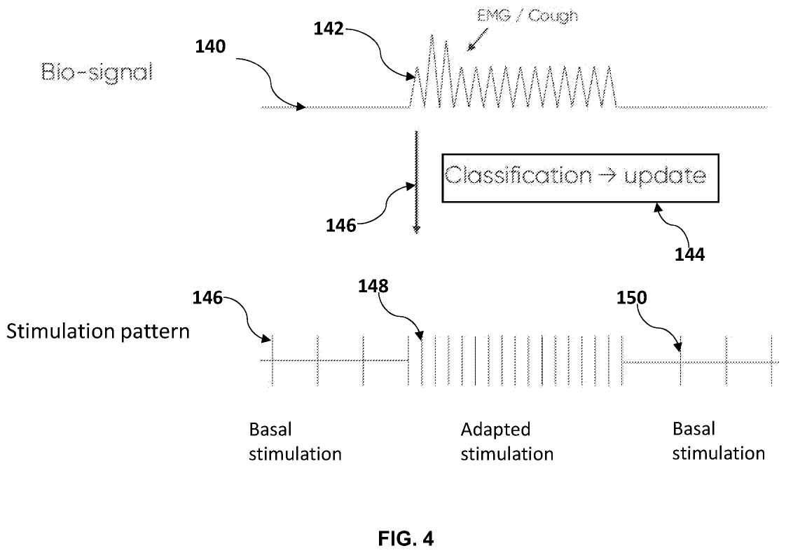

FIG. 4 depicts the sensed myoelectric EMG signal and a corresponding adapted electrical stimulation (comprising a stimulation pattern) provided by the devices disclosed herein. In some embodiments, the myoelectric EMG signal (Bio-signal) 140 comprises electrical fluctuations 142 corresponding to contractile activity of the muscle near the sensor. In some embodiments, the electrical fluctuations 142 of a sensed an electromyography (EMG) reading are associated with a cough, sudden movement, change in inertia, or any combination thereof. In some embodiments, the bio-signal captured by the sensor may be analyzed and classified 144 to identify any parameters associated with an episode of pain, like parameters associated with a cough or a sudden movement, from the EMG reading. In some embodiments, the identified episode of pain may initiate a process 146 by which the implantable electric stimulator delivers an adapted electric stimulation comprising a stimulation pattern 148 with a stimulator electrode to a one or more pudendal nerve to prevent or reduce pain of the individual. In some embodiments, the stimulator electrode comprising one or more stimulators delivers an adapted electrical stimulation 122 that is configured to supplement the innate reflex detected by the one or more sensors 120 to account for the episode of pain.

In some embodiments, the electric stimulator comprises a width and length to be readily implantable in a patient. In some embodiments, the electric stimulator comprises a width and length to accommodate circuitry and/or other system level components, described elsewhere herein.

In some embodiments, the electric stimulator comprises a width and length to provide sufficient space for a battery, where the battery comprises a lifetime after which the battery may be replaced. In some embodiments, the battery lifetime comprises about 5 years to about 15 years. In some embodiments, the battery lifetime comprises about 5 years to about 6 years, about 5 years to about 7 years, about 5 years to about 8 years, about 5 years to about 9 years, about 5 years to about 10 years, about 5 years to about 11 years, about 5 years to about 12 years, about 5 years to about 13 years, about 5 years to about 14 years, about 5 years to about 15 years, about 6 years to about 7 years, about 6 years to about 8 years, about 6 years to about 9 years, about 6 years to about 10 years, about 6 years to about 11 years, about 6 years to about 12 years, about 6 years to about 13 years, about 6 years to about 14 years, about 6 years to about 15 years, about 7 years to about 8 years, about 7 years to about 9 years, about 7 years to about 10 years, about 7 years to about 11 years, about 7 years to about 12 years, about 7 years to about 13 years, about 7 years to about 14 years, about 7 years to about 15 years, about 8 years to about 9 years, about 8 years to about 10 years, about 8 years to about 11 years, about 8 years to about 12 years, about 8 years to about 13 years, about 8 years to about 14 years, about 8 years to about 15 years, about 9 years to about 10 years, about 9 years to about 11 years, about 9 years to about 12 years, about 9 years to about 13 years, about 9 years to about 14 years, about 9 years to about 15 years, about 10 years to about 11 years, about 10 years to about 12 years, about 10 years to about 13 years, about 10 years to about 14 years, about 10 years to about 15 years, about 11 years to about 12 years, about 11 years to about 13 years, about 11 years to about 14 years, about 11 years to about 15 years, about 12 years to about 13 years, about 12 years to about 14 years, about 12 years to about 15 years, about 13 years to about 14 years, about 13 years to about 15 years, or about 14 years to about 15 years. In some embodiments, the battery lifetime comprises about 5 years, about 6 years, about 7 years, about 8 years, about 9 years, about 10 years, about 11 years, about 12 years, about 13 years, about 14 years, or about 15 years. In some embodiments, the battery lifetime comprises at least about 5 years, about 6 years, about 7 years, about 8 years, about 9 years, about 10 years, about 11 years, about 12 years, about 13 years, or about 14 years. In some embodiments, the battery lifetime comprises at most about 6 years, about 7 years, about 8 years, about 9 years, about 10 years, about 11 years, about 12 years, about 13 years, about 14 years, or about 15 years.

In some embodiments, the electric stimulator battery may require charging once in about 1 day to about 12 days. In some embodiments, the electric stimulator battery may require charging once in about 1 day to about 2 days, about 1 day to about 3 days, about 1 day to about 4 days, about 1 day to about 5 days, about 1 day to about 6 days, about 1 day to about 7 days, about 1 day to about 8 days, about 1 day to about 9 days, about 1 day to about 10 days, about 1 day to about 11 days, about 1 day to about 12 days, about 2 days to about 3 days, about 2 days to about 4 days, about 2 days to about 5 days, about 2 days to about 6 days, about 2 days to about 7 days, about 2 days to about 8 days, about 2 days to about 9 days, about 2 days to about 10 days, about 2 days to about 11 days, about 2 days to about 12 days, about 3 days to about 4 days, about 3 days to about 5 days, about 3 days to about 6 days, about 3 days to about 7 days, about 3 days to about 8 days, about 3 days to about 9 days, about 3 days to about 10 days, about 3 days to about 11 days, about 3 days to about 12 days, about 4 days to about 5 days, about 4 days to about 6 days, about 4 days to about 7 days, about 4 days to about 8 days, about 4 days to about 9 days, about 4 days to about 10 days, about 4 days to about 11 days, about 4 days to about 12 days, about 5 days to about 6 days, about 5 days to about 7 days, about 5 days to about 8 days, about 5 days to about 9 days, about 5 days to about 10 days, about 5 days to about 11 days, about 5 days to about 12 days, about 6 days to about 7 days, about 6 days to about 8 days, about 6 days to about 9 days, about 6 days to about 10 days, about 6 days to about 11 days, about 6 days to about 12 days, about 7 days to about 8 days, about 7 days to about 9 days, about 7 days to about 10 days, about 7 days to about 11 days, about 7 days to about 12 days, about 8 days to about 9 days, about 8 days to about 10 days, about 8 days to about 11 days, about 8 days to about 12 days, about 9 days to about 10 days, about 9 days to about 11 days, about 9 days to about 12 days, about 10 days to about 11 days, about 10 days to about 12 days, or about 11 days to about 12 days. In some embodiments, the electric stimulator battery may require charging once in about 1 day, about 2 days, about 3 days, about 4 days, about 5 days, about 6 days, about 7 days, about 8 days, about 9 days, about 10 days, about 11 days, or about 12 days. In some embodiments, the electric stimulator battery may require charging once in at least about 1 day, about 2 days, about 3 days, about 4 days, about 5 days, about 6 days, about 7 days, about 8 days, about 9 days, about 10 days, or about 11 days. In some embodiments, the electric stimulator battery may require charging once in at most about 2 days, about 3 days, about 4 days, about 5 days, about 6 days, about 7 days, about 8 days, about 9 days, about 10 days, about 11 days, or about 12 days.

In some embodiments, the electric stimulator comprises a width of about 1 mm to about 50 mm. In some embodiments, the electric stimulator comprises a width of about 1 mm to about 5 mm, about 1 mm to about 10 mm, about 1 mm to about 15 mm, about 1 mm to about 20 mm, about 1 mm to about 25 mm, about 1 mm to about 30 mm, about 1 mm to about 35 mm, about 1 mm to about 40 mm, about 1 mm to about 45 mm, about 1 mm to about 50 mm, about 5 mm to about 10 mm, about 5 mm to about 15 mm, about 5 mm to about 20 mm, about 5 mm to about 25 mm, about 5 mm to about 30 mm, about 5 mm to about 35 mm, about 5 mm to about 40 mm, about 5 mm to about 45 mm, about 5 mm to about 50 mm, about 10 mm to about 15 mm, about 10 mm to about 20 mm, about 10 mm to about 25 mm, about 10 mm to about 30 mm, about 10 mm to about 35 mm, about 10 mm to about 40 mm, about 10 mm to about 45 mm, about 10 mm to about 50 mm, about 15 mm to about 20 mm, about 15 mm to about 25 mm, about 15 mm to about 30 mm, about 15 mm to about 35 mm, about 15 mm to about 40 mm, about 15 mm to about 45 mm, about 15 mm to about 50 mm, about 20 mm to about 25 mm, about 20 mm to about 30 mm, about 20 mm to about 35 mm, about 20 mm to about 40 mm, about 20 mm to about 45 mm, about 20 mm to about 50 mm, about 25 mm to about 30 mm, about 25 mm to about 35 mm, about 25 mm to about 40 mm, about 25 mm to about 45 mm, about 25 mm to about 50 mm, about 30 mm to about 35 mm, about 30 mm to about 40 mm, about 30 mm to about 45 mm, about 30 mm to about 50 mm, about 35 mm to about 40 mm, about 35 mm to about 45 mm, about 35 mm to about 50 mm, about 40 mm to about 45 mm, about 40 mm to about 50 mm, or about 45 mm to about 50 mm. In some embodiments, the electric stimulator comprises a width of about 1 mm, about 5 mm, about 10 mm, about 15 mm, about 20 mm, about 25 mm, about 30 mm, about 35 mm, about 40 mm, about 45 mm, or about 50 mm. In some embodiments, the electric stimulator comprises a width of at least about 1 mm, about 5 mm, about 10 mm, about 15 mm, about 20 mm, about 25 mm, about 30 mm, about 35 mm, about 40 mm, or about 45 mm. In some embodiments, the electric stimulator comprises a width of at most about 5 mm, about 10 mm, about 15 mm, about 20 mm, about 25 mm, about 30 mm, about 35 mm, about 40 mm, about 45 mm, or about 50 mm.

In some embodiments, the electric stimulator comprises a length of about 1 mm to about 50 mm. In some embodiments, the electric stimulator comprises a length of about 1 mm to about 5 mm, about 1 mm to about 10 mm, about 1 mm to about 15 mm, about 1 mm to about 20 mm, about 1 mm to about 25 mm, about 1 mm to about 30 mm, about 1 mm to about 35 mm, about 1 mm to about 40 mm, about 1 mm to about 45 mm, about 1 mm to about 50 mm, about 5 mm to about 10 mm, about 5 mm to about 15 mm, about 5 mm to about 20 mm, about 5 mm to about 25 mm, about 5 mm to about 30 mm, about 5 mm to about 35 mm, about 5 mm to about 40 mm, about 5 mm to about 45 mm, about 5 mm to about 50 mm, about 10 mm to about 15 mm, about 10 mm to about 20 mm, about 10 mm to about 25 mm, about 10 mm to about 30 mm, about 10 mm to about 35 mm, about 10 mm to about 40 mm, about 10 mm to about 45 mm, about 10 mm to about 50 mm, about 15 mm to about 20 mm, about 15 mm to about 25 mm, about 15 mm to about 30 mm, about 15 mm to about 35 mm, about 15 mm to about 40 mm, about 15 mm to about 45 mm, about 15 mm to about 50 mm, about 20 mm to about 25 mm, about 20 mm to about 30 mm, about 20 mm to about 35 mm, about 20 mm to about 40 mm, about 20 mm to about 45 mm, about 20 mm to about 50 mm, about 25 mm to about 30 mm, about 25 mm to about 35 mm, about 25 mm to about 40 mm, about 25 mm to about 45 mm, about 25 mm to about 50 mm, about 30 mm to about 35 mm, about 30 mm to about 40 mm, about 30 mm to about 45 mm, about 30 mm to about 50 mm, about 35 mm to about 40 mm, about 35 mm to about 45 mm, about 35 mm to about 50 mm, about 40 mm to about 45 mm, about 40 mm to about 50 mm, or about 45 mm to about 50 mm. In some embodiments, the electric stimulator comprises a length of about 1 mm, about 5 mm, about 10 mm, about 15 mm, about 20 mm, about 25 mm, about 30 mm, about 35 mm, about 40 mm, about 45 mm, or about 50 mm. In some embodiments, the electric stimulator comprises a length of at least about 1 mm, about 5 mm, about 10 mm, about 15 mm, about 20 mm, about 25 mm, about 30 mm, about 35 mm, about 40 mm, or about 45 mm. In some embodiments, the electric stimulator comprises a length of at most about 5 mm, about 10 mm, about 15 mm, about 20 mm, about 25 mm, about 30 mm, about 35 mm, about 40 mm, about 45 mm, or about 50 mm.

In some embodiments, the electric stimulator comprises a height of about 0.5 mm to about 5.5 mm. In some embodiments, the electric stimulator comprises a height of about 0.5 mm to about 1 mm, about 0.5 mm to about 1.5 mm, about 0.5 mm to about 2 mm, about 0.5 mm to about 2.5 mm, about 0.5 mm to about 3 mm, about 0.5 mm to about 3.5 mm, about 0.5 mm to about 4 mm, about 0.5 mm to about 4.5 mm, about 0.5 mm to about 5 mm, about 0.5 mm to about 5.5 mm, about 1 mm to about 1.5 mm, about 1 mm to about 2 mm, about 1 mm to about 2.5 mm, about 1 mm to about 3 mm, about 1 mm to about 3.5 mm, about 1 mm to about 4 mm, about 1 mm to about 4.5 mm, about 1 mm to about 5 mm, about 1 mm to about 5.5 mm, about 1.5 mm to about 2 mm, about 1.5 mm to about 2.5 mm, about 1.5 mm to about 3 mm, about 1.5 mm to about 3.5 mm, about 1.5 mm to about 4 mm, about 1.5 mm to about 4.5 mm, about 1.5 mm to about 5 mm, about 1.5 mm to about 5.5 mm, about 2 mm to about 2.5 mm, about 2 mm to about 3 mm, about 2 mm to about 3.5 mm, about 2 mm to about 4 mm, about 2 mm to about 4.5 mm, about 2 mm to about 5 mm, about 2 mm to about 5.5 mm, about 2.5 mm to about 3 mm, about 2.5 mm to about 3.5 mm, about 2.5 mm to about 4 mm, about 2.5 mm to about 4.5 mm, about 2.5 mm to about 5 mm, about 2.5 mm to about 5.5 mm, about 3 mm to about 3.5 mm, about 3 mm to about 4 mm, about 3 mm to about 4.5 mm, about 3 mm to about 5 mm, about 3 mm to about 5.5 mm, about 3.5 mm to about 4 mm, about 3.5 mm to about 4.5 mm, about 3.5 mm to about 5 mm, about 3.5 mm to about 5.5 mm, about 4 mm to about 4.5 mm, about 4 mm to about 5 mm, about 4 mm to about 5.5 mm, about 4.5 mm to about 5 mm, about 4.5 mm to about 5.5 mm, or about 5 mm to about 5.5 mm. In some embodiments, the electric stimulator comprises a height of about 0.5 mm, about 1 mm, about 1.5 mm, about 2 mm, about 2.5 mm, about 3 mm, about 3.5 mm, about 4 mm, about 4.5 mm, about 5 mm, or about 5.5 mm. In some embodiments, the electric stimulator comprises a height of at least about 0.5 mm, about 1 mm, about 1.5 mm, about 2 mm, about 2.5 mm, about 3 mm, about 3.5 mm, about 4 mm, about 4.5 mm, or about 5 mm. In some embodiments, the electric stimulator comprises a height of at most about 1 mm, about 1.5 mm, about 2 mm, about 2.5 mm, about 3 mm, about 3.5 mm, about 4 mm, about 4.5 mm, about 5 mm, or about 5.5 mm.

In some embodiments, the electric stimulator comprises a mass of about 1 g to about 18 g. In some embodiments, the electric stimulator comprises a mass of about 1 g to about 3 g, about 1 g to about 6 g, about 1 g to about 8 g, about 1 g to about 10 g, about 1 g to about 12 g, about 1 g to about 14 g, about 1 g to about 16 g, about 1 g to about 17 g, about 1 g to about 18 g, about 3 g to about 6 g, about 3 g to about 8 g, about 3 g to about 10 g, about 3 g to about 12 g, about 3 g to about 14 g, about 3 g to about 16 g, about 3 g to about 17 g, about 3 g to about 18 g, about 6 g to about 8 g, about 6 g to about 10 g, about 6 g to about 12 g, about 6 g to about 14 g, about 6 g to about 16 g, about 6 g to about 17 g, about 6 g to about 18 g, about 8 g to about 10 g, about 8 g to about 12 g, about 8 g to about 14 g, about 8 g to about 16 g, about 8 g to about 17 g, about 8 g to about 18 g, about 10 g to about 12 g, about 10 g to about 14 g, about 10 g to about 16 g, about 10 g to about 17 g, about 10 g to about 18 g, about 12 g to about 14 g, about 12 g to about 16 g, about 12 g to about 17 g, about 12 g to about 18 g, about 14 g to about 16 g, about 14 g to about 17 g, about 14 g to about 18 g, about 16 g to about 17 g, about 16 g to about 18 g, or about 17 g to about 18 g. In some embodiments, the electric stimulator comprises a mass of about 1 g, about 3 g, about 6 g, about 8 g, about 10 g, about 12 g, about 14 g, about 16 g, about 17 g, or about 18 g. In some embodiments, the electric stimulator comprises a mass of at least about 1 g, about 3 g, about 6 g, about 8 g, about 10 g, about 12 g, about 14 g, about 16 g, or about 17 g. In some embodiments, the electric stimulator comprises a mass of at most about 3 g, about 6 g, about 8 g, about 10 g, about 12 g, about 14 g, about 16 g, about 17 g, or about 18 g.

In some embodiments, the electric stimulator may be sterilizable with conventional methods of sterilization used in the medical field, e.g., gas sterilization, steam sterilization, UV sterilization, etc.

In some embodiments, the one or more stimulator electrodes may provide a base electrical stimulation, or also referred herein as basal electrical stimulation, 146 and 150 for an individual experiencing pain. In some embodiments, the base electrical stimulation comprises constant frequency, amplitude, current, or any combination thereof. In some embodiments, the stimulator electrode may provide a temporary electrical stimulation lasting the duration of an episode of exacerbating pain 148 for an individual experiencing chronic pelvic pain. In some embodiments, the stimulator electrode may provide the base electrical stimulation (i.e., an electrical stimulation comprising a basal stimulation pattern) with a temporary activated stimulation (i.e., activation stimulation pattern) lasting the duration of a pain episode.

In some embodiments, the device disclosed herein comprises a non-transitory computer readable medium that includes software. In some embodiments, the software may be configured to record a signal from the one or more sensors. In some embodiments, the software may be configured to process the recorded signal from the one or more sensors to determine whether an electrical stimulation pattern may need to be delivered to the one or more stimulator electrodes innervating the one or more pudendal nerves. In some embodiments, the software may be configured to adjust parameters of the sensor in response to the observed signal. In some embodiments, the recorded signal of the sensor by the software may observe a signal that saturates the sensor's dynamic range. In some embodiments, the gain of the sensor may be adjusted by the software to allow for sufficient monitoring and thresholding of the myoelectric EMG signals of the individual.

### System

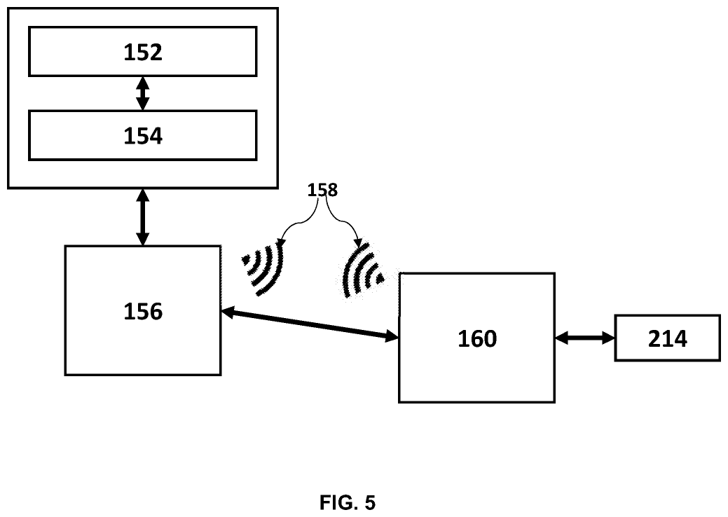

FIG. 5 shows an exemplary embodiment of a system block diagram for the devices and methods described herein with the slow-and fast-adapting algorithms. In some embodiments, the systems disclosed herein comprises a plurality of sub-modules. In some embodiments, the sub-modules comprise: an offline analysis module 152, a clinician control module 154, a patient controller module 156, an implantable module 160, or any combination thereof.

In some embodiments, the offline analysis module 152 comprises a data repository, an analysis software a visualization software, or any combination thereof. In some embodiments, the offline analysis module may be used to retrospectively analyze and graphically visualize an individual's implant performance to prevent or reduce pain of the individual. The off-line analysis module 152 may be programmatically coupled to the clinician control module through an application programming interface (API). In some embodiments, the clinician control module comprises software that may tune or change the electrical stimulation patterns of the individual's electrical stimulation implant.

In some embodiments, the clinical control module 154 comprises stimulation management and device monitor software, stimulation programming map, classification configuration dashboard, streaming data collection dashboard, or any combination thereof. In some embodiments, a health care personnel may assist an individual with an electrical implant by updating or modifying their electrical implant parameters through the clinical control module 154. In some embodiments, the clinical control module may be utilized to initialize an individual's electrical implant after implantation through an USB interface to the patient controller module 156.

In some embodiments, the patient controller module 156 comprises a direct interface to control aspects of their electrical stimulator as described herein. In some embodiments, the patient controller module 156 comprises: medical information and communication band (MICS) communication platform, manual electrical stimulator control, enable or disable algorithm functionality, algorithm patient alerts, an inductive or wired charger for the implantable pulse generator rechargeable battery, or any combination thereof.

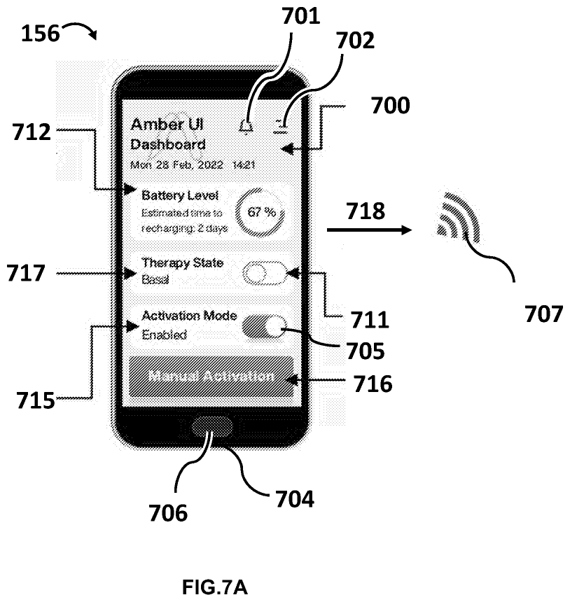

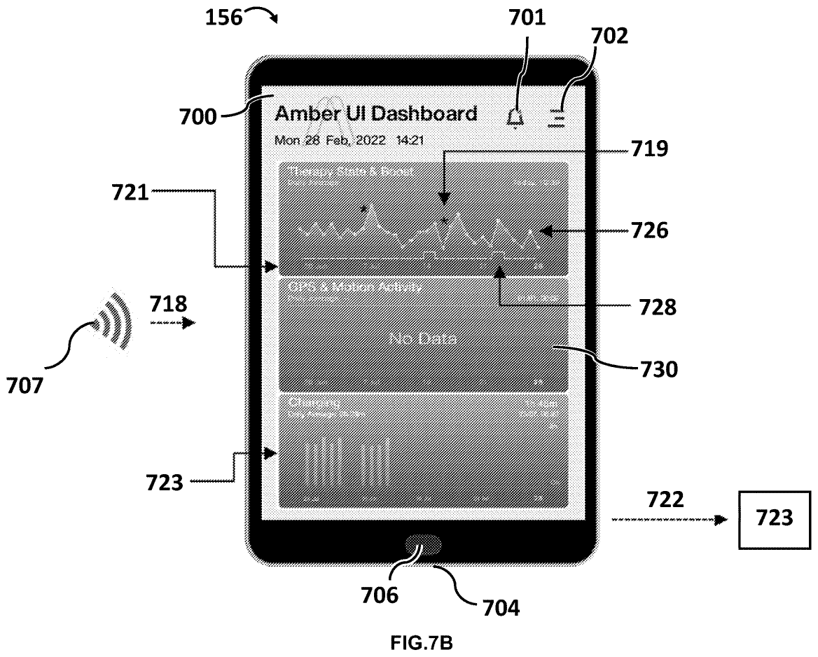

In some embodiments, the patient controller module 156 may be configured to wirelessly and/or inductively charge the implantable pulse generator rechargeable battery with a recharger of the patient controller module 156. In some embodiments, the patient controller module 156 may magnetically couple to the implantable pulse generator 160 from outside the subject's skin. In some embodiments, the magnetic coupling of the controller module 156 to the implantable pulse generator 160 may be made such that the coupling is ergonomic for the subject such that the subject may conduct him/herself as if the controller module 156 is not magnetically coupled to the implantable pulse generator 160. In some embodiments, the patient controller module 156 comprises a battery that may be recharged through wireless inductive charging via the recharger and/or wired charging. In some embodiments, the patient controller module 156 comprises a rechargeable lithium-ion battery. In some embodiments, the recharger comprises one or more inductive coils used when charging the patient controller module 156 and/or when using the patient controller module 156 to charge the implantable pulse generator 160. In some embodiments, the patient controller module, shown as shown in FIG. 7B may be configured 722 to accept additional memory storage 723. In some embodiments, the additional memory storage may be used to transport patient data and/or information between patient/subject and provider.

In some embodiments, the patient controller module 156 may directly or automatically control the implantable pulse generator 160. In some embodiments, the implantable pulse generator 160 comprises a corresponding MICS-telemetry communication platform to that of the MICS communication platform of the patient controller module, enabling the communication between the two devices over an ad hoc Wi-Fi network 158. In some embodiments, the implantable pulse generator 160 may further comprise a three-axis accelerometer biopotential amplifier that may be electrically coupled to one or more electrodes leads 214. In some embodiments the one or more electrode leads comprises one or more stimulator and/or sensors. In some embodiments, a biopotential amplifier may be electrically coupled to a computation sub module comprising a classifier, control policy, real-time clock scheduler, microprocessor, or any combination thereof. In some embodiments, the biopotential amplifier, classifier, control policy, real-time clock scheduler, and microprocessor, or any combination thereof, may process and interpret detected myoelectric EMG signals in the patient to determine the necessary electrical stimulation pattern provided by the actuator to the one or more stimulator electrodes to prevent an episode of pain in an individual.

In some embodiments, the patient controller module 156 comprises a user interface, ports, indicators, or any combination thereof as seen in FIGS. 7A-7B. The user interface and/or ports of the patient controller module comprises an input charging socket 704, keypad navigation button 706, stimulation indicator, communication indicator, output charging adapter port, battery level indicator in both percentage and number of days 712, manual excitation over-ride button 716, or any combination thereof. In some embodiments, the input charging socket may be configured to accept a USB A, B, and/or C, Firewire, any micro versions thereof, or any combinations thereof. connections. In some embodiments, the patient controller module 156 may be connected to a power converter to through a corresponding cable adapted to the input charging socket to charge the patient controller module 156.

### User Interface

The patient controller module 156 comprises a user interface 700 where the user interface comprises one or more user interface objects (701, 702, 705, 711, 712, 715, 716, 717, 721, 730, 723), and/or views as seen in FIG. 7A-7B. In some embodiments, the user interface 700 comprises a touch screen display configured to receive touch or pressing input from a user, patient, and/or medical care personnel. In some embodiments, a user, patient, and/or medical care personnel may press and/or interact with button 716 based mixed graphic and text indicators, and/or switch mixed graphic and text indicators (705, 711). In some embodiments, the user, patient, and/or medical care personnel may double tap a user interface object and/or a physical interface e.g., a surface or button of the implanted pulse generator to enable an emergency state. In some cases, the physical interface may comprise an interface of the implanted pulse generator that may be touched or physically pressed and/or tapped by the individual with the implant. In some embodiments, the emergency state may enable the implanted stimulator to provide electrical stimulation immediately in response to the double tap command. In some embodiments, a parameter or setting value of the user interface objects may be modified and/or changed by tilting the patient controller module. In some embodiments, tilting the patient controller module in a first direction may increase the parameter and/or setting value of the user interface object whereas tilting the patient controller module in a second direction opposite the first direction may decrease the parameter and/or setting value.

In some embodiments, the user interface between devices such as smart phones and tablets or other personal computing device comprises a scaled version of the user interface. In some embodiments, the different user interface views e.g., the view shown in FIG. 7A and FIG. 7B may display varying user interface objects. In some embodiments, the user interface objects comprise one or more buttons 716, switches (711, 716), and/or graphical or image based representation of data (721, 730, 723).

In some embodiments, users may customize the user interface object with a selection of one or more user interface objects (e.g., buttons, switch button to enable various device operation modes, graphical displays of device data, etc.). In some embodiments, the user views may be a predetermine set of views with set user interface object. In some embodiments, the user may customize and/or create one or more views accessible by a menu icon 702. In some embodiments, the menu icon 702 may be configured to display one or more submenu options. In some embodiments, the one or more submenu options comprises personal identification, account information, device registration, customer support, or any combination thereof submenus. In some embodiments, one submenu comprises information of how to connect the device platform to pre-existing health care providers. In some embodiments, the user interface comprises a notification object 701. The notification object may display a unique or highlighted state if a particular notification of device performance, detection of an episode of pain, or any combination thereof is to be provided to the user of the device. A user may interact with a press the notification object to view, in the form of a pop-up dialogue, the particular notification.

In some embodiments, the one or more user interface objects comprises text and/or mixed text and vector objects representations of the various API function calls and/or sub-user interface views, as seen in FIGS. 7A-7B. In some embodiments, the user interface comprises mixed text and vector objects that permit the subject or user to activate 705 or de-activate 711 electrical stimulation of the device 716, adjust device parameters 715, indicate therapy state 717, view device measured EMG signals 721, view stimulator electrode electrical signal characteristics (e.g., frequency, amplitude, pulse width, etc.) delivered, view a medical portal to submit user data to a health care provider, log resulting episodes of pain 719 overlaid on top of measured ENG/EMG signals, or any combination thereof. In some embodiments, the user interface may further comprise a battery 712 and wireless communication connectivity indicator for the users and/or subjects to visualize patient controller module 156 operating properties.

In some embodiments, the keypad navigation button 706 may be configured to navigate between various user interface views e.g., the user interface views provided in FIG. 7A and FIG. 7B

In some embodiments, the patient controller module 156 comprises visual indicators (715, 717, 712), configured to indicate whether the implanted electrical stimulator is outputting electrical stimulation and/or the presence or lack thereof connectivity with a second or third device. In some embodiments, the patient controller module comprises a device adjustment parameter, where the device adjustment parameter comprises a stimulation indicator, or an activation of a stimulation mode 715. In some embodiments, the stimulation indicator may be in electrical communication with a processor, described elsewhere herein, configured to display a visual indicator when the stimulator is providing an electrical stimulation to a subject. In some embodiments, the patient controller module comprises a connectivity indicator. In some embodiments, the connectivity indicator may be in electrical communication with a processor, described elsewhere herein, and configured to provide a visual indicator when the patient controller module is connected to one or more discrete devices, data servers, local WIFI or ad-hoc WIFI networks, Bluetooth, medical implant communication system (MICS), or any combination thereof. In some embodiments, the connectivity indicator may indicate the wireless connection with the implanted electrical stimulator. In some embodiments, the connectivity indicator comprises one or more states. In some embodiments, a first state my comprise a solid image indicator, where such a solid image indicator may notify a user, subject, individual, and/or health care personnel, a successfully established communication pairing between the patient controller module and a third device, server, etc. A second state comprises a flashing image indicator, where such a flashing image indicator indicates a paired communication state between the patient controller module and a third device, server, etc. In some embodiments, the image indicator comprises the universal symbol for Bluetooth that may be observed on smart devices and/or devices with Bluetooth connectivity. In some embodiments, the image indicator comprises a graphic of the universal symbol indicator for Wi-Fi (e.g., a quarter circle of concentric rings) seen commonly on smart devices and/or devices with Wi-Fi connectivity.

In some embodiments, device data (e.g., EMG/ENG, accelerometer, gyroscopic, magnetometer, 3-D spatial movement, global positioning system (GPS) data, or any combination thereof) may be transmitted 718 over Wi-Fi, Bluetooth, MICS, or other ad-hoc networks between one or more devices, as described elsewhere herein.

FIG. 7B shows a different user interface view than that of the FIG. 7A. In some embodiments, the user interface of FIG. 7B comprises one or more user interface objects (721, 730, 723), where each user interface object displays device data received 718 through wireless transmission 707 as described above. In some embodiments, one of the user interface objects comprises a graphical therapy object 721. The graphical therapy object may display detected EMG/ENG signals 726 and corresponding stimulation profiles 728. In some embodiments, the graphical therapy object may display leak events 719 of where the user indicated an episode of pain but where the device did not provide stimulation. Another user interface object comprises a GPS and motion activity object 730. In some embodiments, the motion activity object 730 may display GPS and motion data of the subject over time. The GPS and motion data may be useful considerations when improving the classifier described elsewhere herein. Another user interface object comprises a charging indicator object 723. In some embodiments, the charging indicator object may display charge capacitance of the implanted stimulator over a period of time. In some embodiments, the charging indicator object may be used to monitor the health of the battery of the implanted stimulator. A user interacting with the display view shown in FIG. 7B may pinch, swipe, or otherwise interact with the data of each user interface object (721, 730, 723) to view other temporal regions of data or to zoom in on a particular scale of a measurement. In some embodiments, through the menu object 702 a user and/or subject may export their medical data to one or more provides.

### Method of Treating Pain

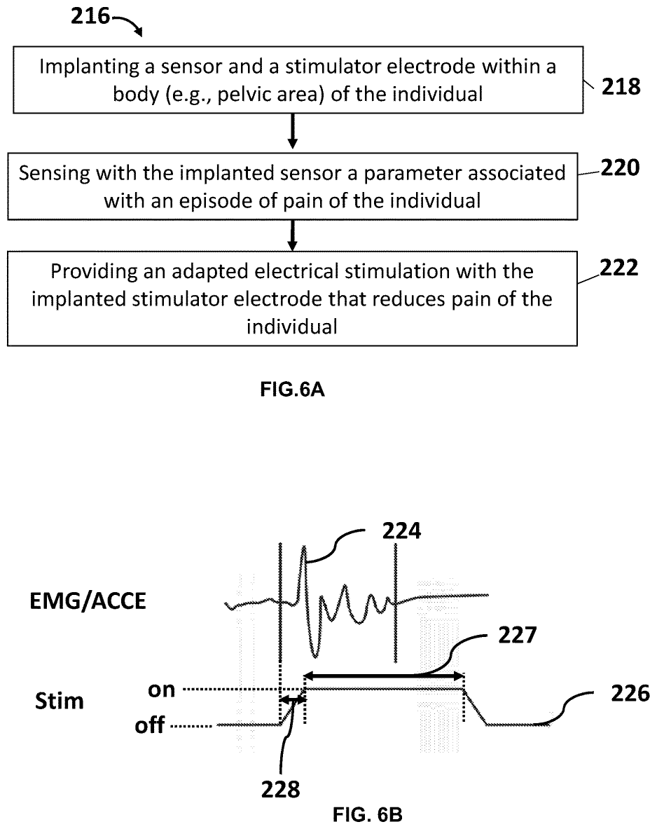

FIG. 6A illustrates a workflow of a method 216 of reducing pain of an individual. The method comprises the steps of (a) implanting a sensor and a stimulator electrode within a body (e.g., pelvic area) of the individual 218; (b) sensing with the implanted sensor a parameter associated with an episode of pain of the individual 220; and (c) providing an adapted electrical stimulation with the implanted stimulator electrode that reduces pain of the individual 222. The methods described herein prevent or reduce pain associated with an episode of pain of the individual by providing an adapted electrical stimulation with a stimulator electrode implanted in the individual. In some embodiments, the sensor is implanted in the body of the individual. In some embodiments the sensor is implanted in the pelvic area of the individual.

In some embodiments, the method comprises a step of providing a base electrical stimulation. In some embodiments, the base electrical stimulation is provided at a lower intensity level than the adapted electrical stimulation. In some embodiments, the base electrical stimulation is provided at a different intensity level than the adapted electrical stimulation. In some embodiments, the base electrical stimulation provided reduces pain associated with constant or near-constant pain associated with chronic pelvic pain. In some embodiments, the intensity or duration of the adapted electrical stimulation provided varies according to the parameter that is sensed. In some embodiments, the parameter indicates that at least one of the amplitude, frequency, or intensity of the basal electrical stimulation must be increased to prevent or reduce pain of the individual. In some embodiments, the adapted electrical stimulation boosts (i.e., increases at least one of the amplitude, frequency, or intensity) of the base electrical stimulation. In some embodiments, the time period between sensing a parameter 224 and providing an electrical stimulation 226 may be described as a response time 228, as shown in FIG. 6B. In some embodiments, the stimulation may be provided for a duration of stimulation 227.

In some embodiments, the duration of stimulation 227 comprises about 1 second to about 30 seconds. In some embodiments, the duration of stimulation 227 comprises about 1 second to about 2 seconds, about 1 second to about 3 seconds, about 1 second to about 4 seconds, about 1 second to about 5 seconds, about 1 second to about 10 seconds, about 1 second to about 12 seconds, about 1 second to about 14 seconds, about 1 second to about 16 seconds, about 1 second to about 20 seconds, about 1 second to about 25 seconds, about 1 second to about 30 seconds, about 2 seconds to about 3 seconds, about 2 seconds to about 4 seconds, about 2 seconds to about 5 seconds, about 2 seconds to about 10 seconds, about 2 seconds to about 12 seconds, about 2 seconds to about 14 seconds, about 2 seconds to about 16 seconds, about 2 seconds to about 20 seconds, about 2 seconds to about 25 seconds, about 2 seconds to about 30 seconds, about 3 seconds to about 4 seconds, about 3 seconds to about 5 seconds, about 3 seconds to about 10 seconds, about 3 seconds to about 12 seconds, about 3 seconds to about 14 seconds, about 3 seconds to about 16 seconds, about 3 seconds to about 20 seconds, about 3 seconds to about 25 seconds, about 3 seconds to about 30 seconds, about 4 seconds to about 5 seconds, about 4 seconds to about 10 seconds, about 4 seconds to about 12 seconds, about 4 seconds to about 14 seconds, about 4 seconds to about 16 seconds, about 4 seconds to about 20 seconds, about 4 seconds to about 25 seconds, about 4 seconds to about 30 seconds, about 5 seconds to about 10 seconds, about 5 seconds to about 12 seconds, about 5 seconds to about 14 seconds, about 5 seconds to about 16 seconds, about 5 seconds to about 20 seconds, about 5 seconds to about 25 seconds, about 5 seconds to about 30 seconds, about 10 seconds to about 12 seconds, about 10 seconds to about 14 seconds, about 10 seconds to about 16 seconds, about 10 seconds to about 20 seconds, about 10 seconds to about 25 seconds, about 10 seconds to about 30 seconds, about 12 seconds to about 14 seconds, about 12 seconds to about 16 seconds, about 12 seconds to about 20 seconds, about 12 seconds to about 25 seconds, about 12 seconds to about 30 seconds, about 14 seconds to about 16 seconds, about 14 seconds to about 20 seconds, about 14 seconds to about 25 seconds, about 14 seconds to about 30 seconds, about 16 seconds to about 20 seconds, about 16 seconds to about 25 seconds, about 16 seconds to about 30 seconds, about 20 seconds to about 25 seconds, about 20 seconds to about 30 seconds, or about 25 seconds to about 30 seconds. In some embodiments, the duration of stimulation 227 comprises about 1 second, about 2 seconds, about 3 seconds, about 4 seconds, about 5 seconds, about 10 seconds, about 12 seconds, about 14 seconds, about 16 seconds, about 20 seconds, about 25 seconds, or about 30 seconds. In some embodiments, the duration of stimulation 227 comprises at least about 1 second, about 2 seconds, about 3 seconds, about 4 seconds, about 5 seconds, about 10 seconds, about 12 seconds, about 14 seconds, about 16 seconds, about 20 seconds, or about 25 seconds. In some embodiments, the duration of stimulation 227 comprises at most about 2 seconds, about 3 seconds, about 4 seconds, about 5 seconds, about 10 seconds, about 12 seconds, about 14 seconds, about 16 seconds, about 20 seconds, about 25 seconds, or about 30 seconds.

In some embodiments the response time 228 comprises about 60 μs to about 100 μs. In some embodiments the response time 228 comprises about 60 μs to about 65 μs, about 60 μs to about 70 μs, about 60 μs to about 75 μs, about 60 μs to about 80 μs, about 60 μs to about 85 μs, about 60 μs to about 90 μs, about 60 μs to about 95 μs, about 60 μs to about 100 μs, about 65 μs to about 70 μs, about 65 μs to about 75 μs, about 65 μs to about 80 μs, about 65 μs to about 85 μs, about 65 μs to about 90 μs, about 65 μs to about 95 μs, about 65 μs to about 100 μs, about 70 μs to about 75 μs, about 70 μs to about 80 μs, about 70 μs to about 85 μs, about 70 μs to about 90 μs, about 70 μs to about 95 μs, about 70 μs to about 100 μs, about 75 μs to about 80 μs, about 75 μs to about 85 μs, about 75 μs to about 90 μs, about 75 μs to about 95 μs, about 75 μs to about 100 μs, about 80 μs to about 85 μs, about 80 μs to about 90 μs, about 80 μs to about 95 μs, about 80 μs to about 100 μs, about 85 μs to about 90 μs, about 85 μs to about 95 μs, about 85 μs to about 100 μs, about 90 μs to about 95 μs, about 90 μs to about 100 μs, or about 95 μs to about 100 μs. In some embodiments the response time 228 comprises about 60 μs, about 65 μs, about 70 μs, about 75 μs, about 80 μs, about 85 μs, about 90 μs, about 95 μs, or about 100 μs. In some embodiments the response time 228 comprises at least about 60 μs, about 65 μs, about 70 μs, about 75 μs, about 80 μs, about 85 μs, about 90 μs, or about 95 μs. In some embodiments the response time 228 comprises at most about 65 μs, about 70 μs, about 75 μs, about 80 μs, about 85 μs, about 90 μs, about 95 μs, or about 100 μs. Through the development of iteratively trained machine learning classifiers the response time may be minimized, and improved pain reduction or prevention may be realized.

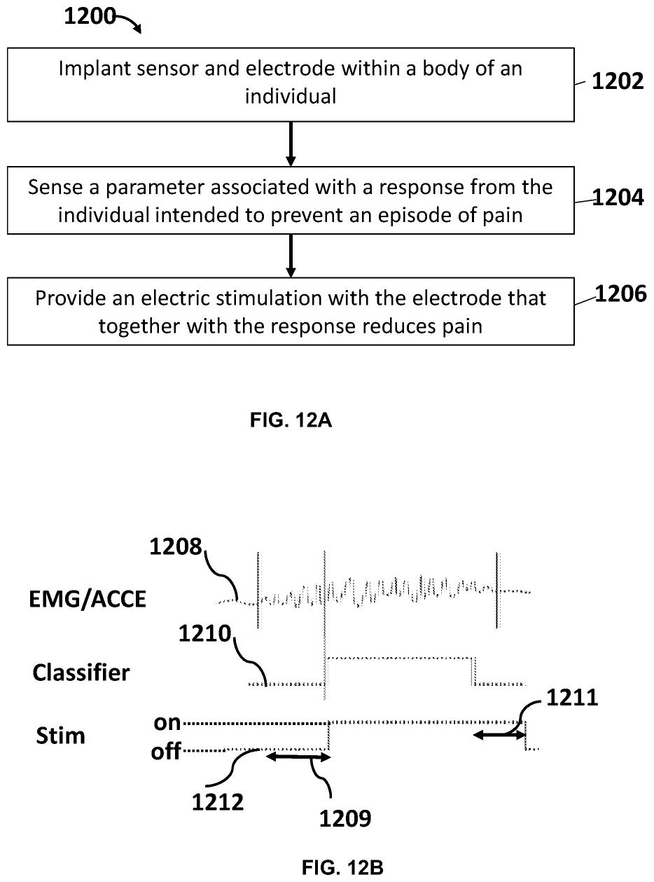

FIGS. 12A-12B illustrate a workflow of a method 1200 for preventing an episode of pain in an individual. The method comprises the steps of (a) implanting a sensor and stimulator electrode within a body of an individual 1202; (b) sensing a parameter associated with pain from the individual, where the response is individual-induced stimulus 1204; and (c) providing an electric stimulation with the stimulator electrode to reduce or prevent the episode of pain 1206. In some embodiments, the user induced stimulus may be intended to prevent an episode of pain. As shown graphically in FIG. 12B, a detected EMG signal 1208 may be analyzed by a classifier 1210, described elsewhere herein, to determine when the EMG, ENG, accelerometer, gyroscope, magnetometer, pressure sensor signals or any combination thereof signals have crossed a pre-determined threshold, described elsewhere herein. Once the classifier 1210 has determined that the signal 1208 does represent an episode of pain, the processor, described elsewhere herein, may enable stimulation 1212 to prevent the episode of pain from occurring. In some embodiments, stimulation 1212 comprises an extension of stimulation 1211 that may extend beyond the time the classifier 1210 determines there to be an episode of pain. In some embodiments the extension of stimulation 1211 comprises a duration of time equal to the duration the subject continues to purposefully or with intent produce a muscle contraction.

In some embodiments, the extension of stimulation comprises about 1 s to about 30 s. In some embodiments the extension of stimulation comprises about 1 s to about 3 s, about 1 s to about 5 s, about 1 s to about 8 s, about 1 s to about 10 s, about 1 s to about 12 s, about 1 s to about 15 s, about 1 s to about 18 s, about 1 s to about 20 s, about 1 s to about 22 s, about 1 s to about 24 s, about 1 s to about 30 s, about 3 s to about 5 s, about 3 s to about 8 s, about 3 s to about 10 s, about 3 s to about 12 s, about 3 s to about 15 s, about 3 s to about 18 s, about 3 s to about 20 s, about 3 s to about 22 s, about 3 s to about 24 s, about 3 s to about 30 s, about 5 s to about 8 s, about 5 s to about 10 s, about 5 s to about 12 s, about 5 s to about 15 s, about 5 s to about 18 s, about 5 s to about 20 s, about 5 s to about 22 s, about 5 s to about 24 s, about 5 s to about 30 s, about 8 s to about 10 s, about 8 s to about 12 s, about 8 s to about 15 s, about 8 s to about 18 s, about 8 s to about 20 s, about 8 s to about 22 s, about 8 s to about 24 s, about 8 s to about 30 s, about 10 s to about 12 s, about 10 s to about 15 s, about 10 s to about 18 s, about 10 s to about 20 s, about 10 s to about 22 s, about 10 s to about 24 s, about 10 s to about 30 s, about 12 s to about 15 s, about 12 s to about 18 s, about 12 s to about 20 s, about 12 s to about 22 s, about 12 s to about 24 s, about 12 s to about 30 s, about 15 s to about 18 s, about 15 s to about 20 s, about 15 s to about 22 s, about 15 s to about 24 s, about 15 s to about 30 s, about 18 s to about 20 s, about 18 s to about 22 s, about 18 s to about 24 s, about 18 s to about 30 s, about 20 s to about 22 s, about 20 s to about 24 s, about 20 s to about 30 s, about 22 s to about 24 s, about 22 s to about 30 s, or about 24 s to about 30 s. In some embodiments the extension of stimulation 1211 comprises about 1 s, about 3 s, about 5 s, about 8 s, about 10 s, about 12 s, about 15 s, about 18 s, about 20 s, about 22 s, about 24 s, or about 30 s. In some embodiments the extension of stimulation 1211 comprises at least about 1 s, about 3 s, about 5 s, about 8 s, about 10 s, about 12 s, about 15 s, about 18 s, about 20 s, about 22 s, or about 24 s. In some embodiments the extension of stimulation 1211 comprises at most about 3 s, about 5 s, about 8 s, about 10 s, about 12 s, about 15 s, about 18 s, about 20 s, about 22 s, about 24 s, or about 30 s.

In some embodiments, there may be a delay 1209 between the onset of the episode of pain in the raw EMG, ENG, accelerometer, gyroscope, magnetometer, pressure sensor, or any combination thereof signal data, and the onset of the electrical stimulation. Upon training the classifier 1210 on sufficiently large and varied datasets, such delay may be minimized further improving the device performance in prevent episodes of pain. In some embodiments, sensing comprises determining a global positioning system (GPS) location of the individual that in combination with the parameter associated with the response from the individual is used to prevent the episode of pain.

### Ambulatory Assessments After Implantation

In some embodiments, various ambulatory assessments may be taken to determine the effectiveness of the implantation procedure. In some embodiments, the implanted IPG permits telemetric downloading of data (inputs, outputs, and event classification). In some embodiments, the participant may be in an awake ambulatory setting and a series of resting and provoked electrophysiological data may be recorded. In some embodiments, at treatment initiation (24-48 hours post-implant) sensory and motor responses may be determined from the different sensors on the implanted leads. In some embodiments, based upon the responses, the electrodes with the most adequate response may be selected to initiate treatment.

In some embodiments, the patients may be subjected to different physiological events to program the IPG. In some embodiments, these events comprise coughing, Valsalva maneuvers, picking up a 5 kg weight, or any combination thereof. In some embodiments, pelvic floor EMG may be measured with a transvaginal and/or anal probe. In some embodiments urethral pressures may be measured. In some embodiments, 1 hour continuous ‘resting’ recording of inputs and outputs (downloaded by telemetry) may be taken. In some embodiments, recording during controlled participant provoked events, such as coughing, Valsalva, lifting 5 Kg weight, may be obtained. In some embodiments, recording during pelvic floor surface EMG (from transvaginal probe: women only) to correlate inputs from lead vs. surface EMG may be obtained. In some embodiments, patient tolerances of basal stimulation ramping, and actuation parameters may be obtained.

### Definitions

Unless defined otherwise, all terms of art, notations and other technical and scientific terms or terminology used herein are intended to have the same meaning as is commonly understood by one of ordinary skill in the art to which the claimed subject matter pertains. In some embodiments, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining”, “measuring”, “evaluating”, “assessing,” “assaying,” and “analyzing” are often used interchangeably herein to refer to forms of measurement and include determining if an element is present or not (for example, detection). These terms can include quantitative, qualitative, or quantitative and qualitative determinations. Assessing can be relative or absolute. “Detecting the presence of” includes determining the amount of something present, as well as determining whether it is present or absent.

The terms “subject,” “individual,” or “patient” are often used interchangeably herein. A “subject” comprises a biological entity containing expressed genetic materials. In some embodiments, the subject comprises an animal, mammal, or human. In some embodiments, the subject is diagnosed or suspected of being at high risk for at least one of chronic pelvic pain (CPP), an associated condition, or an allied syndrome.

The term “in vivo” is used to describe an event that takes place in a subject's body.

The term “ex vivo” is used to describe an event that takes place outside of a subject's body. An “ex vivo” assay is not performed on a subject. Rather, it is performed upon a sample separate from a subject. An example of an “ex vivo” assay performed on a sample is an “in vitro” assay.

As used herein, the term ‘about’ a number refers to that number plus or minus 10% of that number. The term ‘about’ a range refers to that range minus 10% of its lowest value and plus 10% of its greatest value.

As used herein, the terms “treatment” or “treating” are used in reference to an intervention regimen for obtaining beneficial or desired results in the recipient. Beneficial or desired results include but are not limited to a therapeutic benefit and/or a prophylactic benefit. A therapeutic benefit may refer to prevention or amelioration of symptoms or of an underlying disorder being treated. Also, a therapeutic benefit can be achieved with prevention or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or a condition, delaying or eliminating the onset of symptoms of a disease or a condition, slowing, halting, or reversing the progression of a disease or a condition, or any combination thereof. For prophylactic benefit, a subject at risk of developing a particular disease or a condition, or to a subject reporting one or more of the physiological symptoms of a disease or a condition may undergo treatment.

As used herein, the term “muscle” refers to a “myocyte”, “muscle cell”, “muscle fiber”, “muscle tissue”, or any combination thereof.

As used herein, the terms “base electrical stimulation” and “basal electrical stimulation” are used interchangeably.

As used herein, the term “sensor” comprises a detection apparatus that is attached to an implanted lead, including an “electrode”, “sensor electrode”, “sensory electrode”, or any combination thereof. In some embodiments, the sensor, as used herein, detects one or more parameters associated with pain in the individual.

As used herein, the term “electrode lead” or “lead” comprises an implantable lead of an electrode that has at least one of a sensing or stimulating component, including a “stimulator”, “stimulator electrode,” “sensor,” “sensor electrode,” or any combination thereof. In some embodiments, the electrode, as used herein, provides an electrical signal to the target site in the individual.

As used herein, the terms “signal” and “bio-signal” are used interchangeably.

The section headings used herein are for organizational purposes only and are not to be construed as limiting the subject matter described.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1: A System Lacking a Closed-Loop Configuration

FIG. 1 shows an exemplary embodiment of an open-loop bioelectronic system comprising an implantable pulse generator. The system delivers a predefined stimulation protocol and does not receive input from the subject, resulting in inadequate pain management.

### Example 2: Reducing Pain of Episodes Associated With Inflammation

The systems, methods, and devices, described herein, reduce the pain of conditions associated with peripheral drive to pain due to clinical or sub-clinical inflammation (e.g. interstitial cystitis, chronic prostatitis, and radiation vaginitis) by stimulating afferents for gating of peripheral nociception (e.g., positive or negative), anodal block, or any combination thereof. In case of interstitial cystitis, pain increases as the bladder increasingly fills, which is often accompanied with an increase in pelvic floor muscle tone. Thus, an episode of pain can be detected using a sensor configured to detect at least one of an EMG, ENG, or pressure signal. An episode of pain can also be detected using a sensor configured to detect a signal associated with a patient actuated response (e.g., the patient taps a button to indicate onset of an episode of pain, transmitting the signal to a processor or a sensor electrically coupled to the processor).

In some embodiments, a basal stimulation is provided to reduce pain associated with inflammation with adapted stimulations being provided after detecting and/or receiving a parameter associated with an episode of exacerbating pain (e.g., bladder filling beyond a threshold). In some embodiments, receiving a parameter associated with increased pelvic floor muscle tone changes the stimulation provided by the implanted electrode from one anatomical site (e.g., pudendal, sacral, and/or pelvic plexus) to both anatomical sites simultaneously (e.g., pudendal, sacral, and/or pelvic plexus). In some embodiments, the stimulation returns to the initial basal stimulation after bladder emptying ends, which may be initiated by a patient response or by pelvic floor sensing (e.g., EMG, ENG) because emptying the bladder is usually accompanied by relaxation of the pelvic floor muscles.

### Example 3: Preventing Pain of Episodes Associated With Nerve Compression

The systems, methods, and devices, described herein, reduce the pain of conditions associated, at least in part, with compression of nerve endings due to striated muscle spasm (e.g., pelvic floor myalgia, coccygodynia, urethral and penile pain syndromes) and/or anatomical compression (e.g., pudendal neuralgia) by modulating motor nerve activity, anodal block, or any combination thereof. In conditions associated with nerve compression, there may be an impact of bladder filling state and/or body posture (e.g., sitting vs lying) that leads to an episode of exacerbating pain. In some embodiments, a GPS signal is used to indicate or predict an episode of exacerbating pain, like when an individual enters a sleeping room or bathroom (i.e., predict that the individual will soon change posture). In some embodiments, a time and/or time-of-day signal are used to predict or indicate the episode of exacerbating pain. In some embodiments, conditions associated with nerve compression are detected by measuring nerve activity (e.g., by detecting an ENG signal of a sacral or pudendal nerve) and/or measuring activity of the muscle surrounding the compressed nerve (e.g., by detecting an EMG signal of the pelvic floor).

### Example 4: Preventing and Reducing Pain of Episodes Associated With Dysregulated Visceral Smooth Muscle Activity

The systems, methods, and devices, described herein, reduce the pain of conditions mainly associated with dysregulated visceral smooth muscle activity (e.g. painful bladder syndrome, proctalgia fugax) by modulating motor activity to affect organ smooth muscle activity, afferent gate control, anodal block, or any combination thereof.

### Example 6: Preventing and Reducing Pain of Episodes Characterized As Neuropathic Pain

The systems, methods, and devices, described herein, reduce the pain of conditions associated, at least in part, with conditions mainly characterized as neuropathic pain (e.g. vulvodynia, vestibulodynia, phantom rectum syndrome, proctalgia fugax) by afferent gating of spinal activity.

### Example 5: EMG Measurement Reproducibility

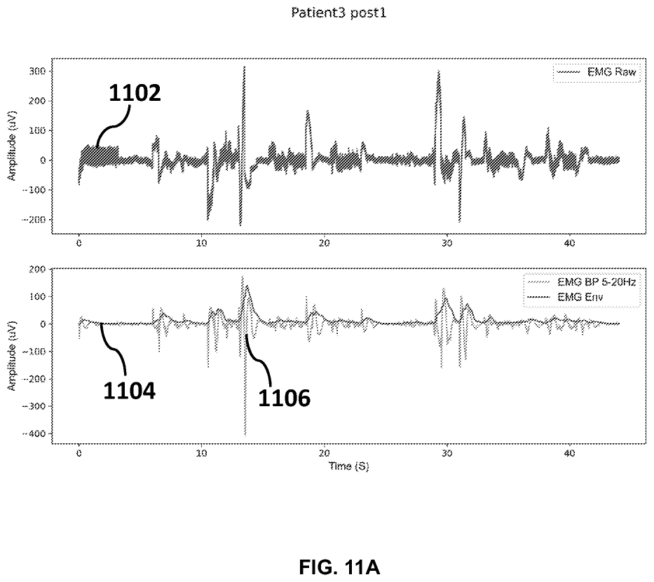

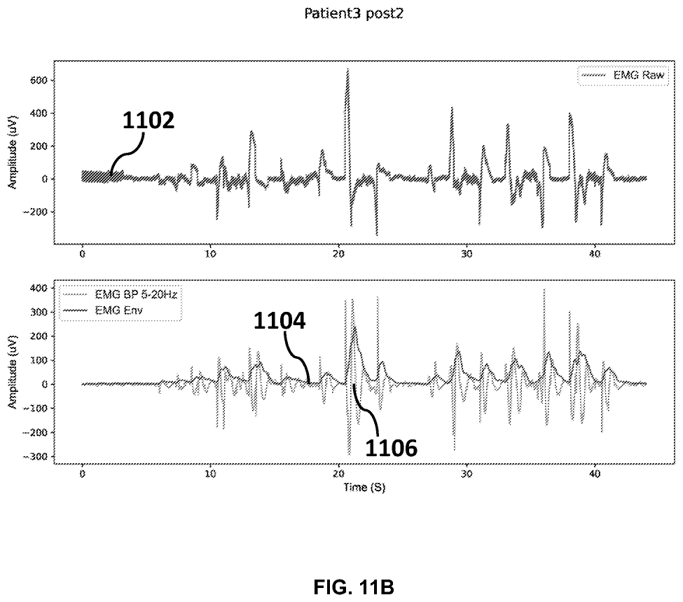

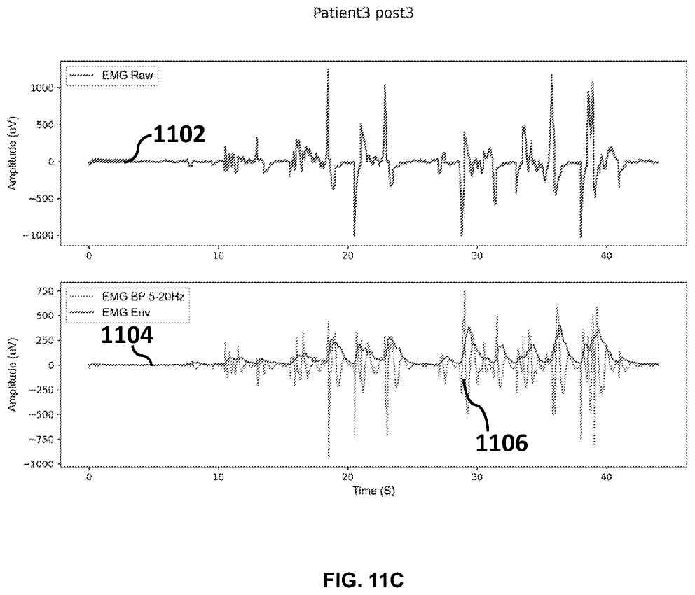

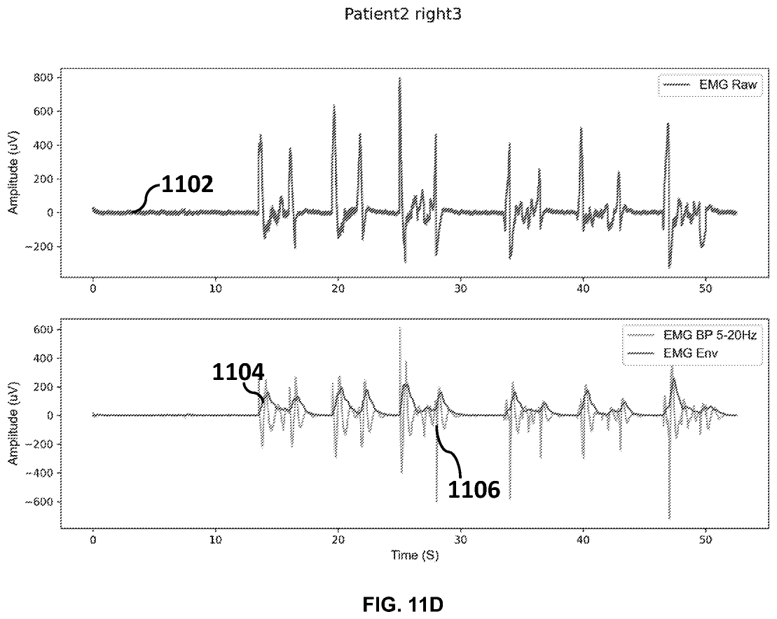

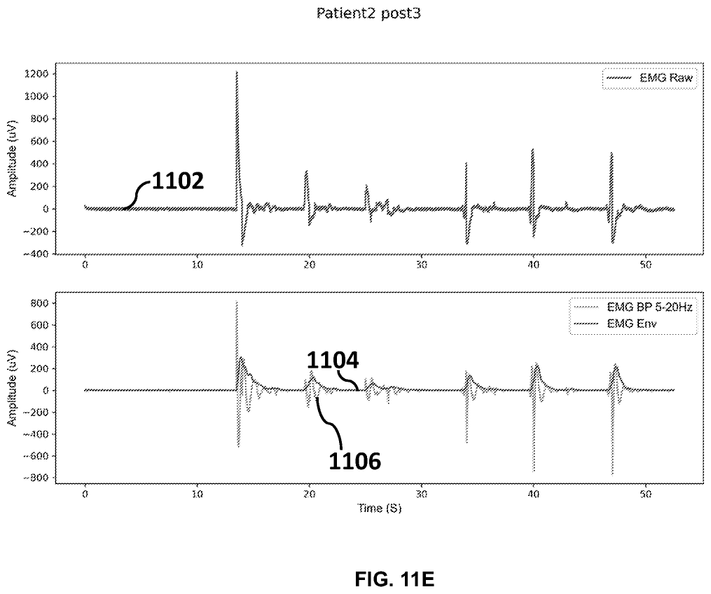

Using the systems, methods, and devices, described herein, patient EMG signals were measured and processed as patients performed muscle contractions, coughing, and Valsalva maneuvers, as can be seen in FIGS. 11A-11E. For each patient, raw EMG data 1102 was recorded and amplified, as described elsewhere herein. Subsequently, the raw EMG data was filtered 1106, rectified, and smoothed 1104.

From the data shown in FIGS. 11A-11C, where a single patient iteratively performed the same sequence of muscle contractions three times, a distinct group of three signals may be observed in the temporal EMG data. Such a finding supports the reproducibility of the sensors of the devices and systems described herein and the potential ability of a trained classifier to distinguish EMG signals temporally. Similarly, data for a different patient shown in FIGS. 11D-11E shows similar results as the patient in FIGS. 11A-11C.

### Example 6: Treating Unilateral Pain

The systems, methods, and/or devices, described elsewhere herein, may be used to treat an individual's unilateral or predominately unilateral pain (e.g., left sided pelvic pudendal neuralgia). To treat the unilateral or predominantly unilateral pain one or more electrode leads (e.g., one or more stimulation or sensing electrodes), described elsewhere herein, are implanted at or adjacent to the left pudendal nerve. Alternatively, a first electrode of the one or more electrodes is implanted on the left pudendal nerve, and a second electrode of the one or more electrodes is implanted at different left sided nerve target such as the sacral nerve or pelvic autonomic plexus. A pulse generator is implanted in the individual and electrically coupled to the one or more electrode leads. The implanted device is then configured by medical care personnel (e.g., an attending physician, nurse, or operating theater room medical support staff) based at least on the individual's known unilateral pain.

After a period of time subsequent to implantation of the one or more electrodes and stimulator, the individual is provided one or more surveys and/or questionaries, e.g., numeric pain rating scale (NPRS), described elsewhere herein, to determine the clinical outcome of the implanted device and improvements to the individual's unilateral pain. A positive and significant clinical outcome of reducing unilateral pain will be reflected in the individual obtaining a NPRS score and/or a VAS rating after receiving treatment that is less than a NPRS score and/or a VAS rating obtained by the individual prior to receiving the implant and pain treatment.

### Example 7: Treating Bilateral Pain

The systems, methods, and/or devices, described elsewhere herein, may be used to treat an individual's bilateral or predominantly bilateral pain. To treat the bilateral or predominantly bilateral pain one or more electrode leads (e.g., one or more stimulation or sensing electrodes), described elsewhere herein, are implanted on the left and right pudendal nerves, both sacral nerves, nerves of the pelvic plexus, or any combination thereof. A pulse generator is implanted in the individual and electrically coupled to the one or more electrode leads. The implanted device is then configured by medical care personnel (e.g., an attending physician, nurse, or operating theater room medical support staff) based at least on the individual's known bilateral pain.

After a period of time subsequent to implantation of the one or more electrodes and stimulator, the individual is provided one or more surveys and/or questionaries, e.g., numeric pain rating scale (NPRS), described elsewhere herein, to determine the clinical outcome of the implanted device and improvements to the individual's bilateral pain. A positive and significant clinical outcome of reducing bilateral pain will be reflected in the individual obtaining a NPRS score and/or a VAS rating after receiving treatment that is less than a NPRS score and/or a VAS rating obtained by the individual prior to receiving the implant and pain treatment.

### Example 8: Treating Bladder Pain Syndrome

The systems, methods, and/or devices, described elsewhere herein, may be used to treat an individual's bladder pain syndrome. To treat bladder pain syndrome for an individual exhibiting primarily urinary incontinence and bladder pain, one or more electrode leads (e.g., one or more stimulation or sensing electrodes), described elsewhere herein, are implanted bilaterally on pudendal nerves to reduce pain and stabilize bladder functions. To treat bladder pain of an individual who has bladder emptying problems, the one or more electrode leads, are implanted at or adjacent a pudendal nerve and a pelvic autonomic nerve to reduce pain and facilitate bladder emptying. A pulse generator is implanted in the individual and electrically coupled to the one or more electrode leads in either case as described above. The implanted device is then configured by medical care personnel (e.g., an attending physician, nurse, or operating theater room medical support staff) based at least on the individual's bladder pain syndrome.

After a period of time subsequent to implantation of the one or more electrodes and stimulator, the individual is provided one or more surveys and/or questionaries, e.g., numeric pain rating scale (NPRS), described elsewhere herein, to determine the clinical outcome of the implanted device and improvements to the individual's bladder pain. A positive and significant clinical outcome of reducing bladder pain will be reflected in the individual obtaining a NPRS score and/or a VAS rating after receiving treatment that is less than a NPRS score and/or a VAS rating obtained by the individual prior to receiving the implant and pain treatment.

### Example 9: Treating Chronic Anal and/or Perineal Pain

The systems, methods, and/or devices, described elsewhere herein, may be used to treat an individual's chronic anal and/or perineal pain. Such subjects presenting with chronic anal and/or perineal pain may have pain associated with or without defecatory symptoms including symptoms of obstructive defecation and/or fecal incontinence. To an individual's chronic anal and/or perineal pain, one or more electrode leads (e.g., one or more stimulation or sensing electrodes), described elsewhere herein, are implanted bilaterally on pudendal and/or pelvic autonomic nerves to reduce pain and facilitate rectal emptying. A pulse generator is implanted in the individual and electrically coupled to the one or more electrode leads in either case as described above. The implanted device is then configured by medical care personnel (e.g., an attending physician, nurse, or operating theater room medical support staff) based at least on the individual's chronic anal and perineal pain.

After a period of time subsequent to implantation of the one or more electrodes and stimulator, the individual is provided one or more surveys and/or questionaries, e.g., numeric pain rating scale (NPRS), and/or a VAS rating, described elsewhere herein, to determine the clinical outcome of the implanted device and improvements to the individual's chronic anal and/or perineal pain. A positive and significant clinical outcome of reducing chronic anal and perineal pain will be reflected in the individual obtaining a NPRS score and/or a VAS rating after receiving treatment that is less than a NPRS score and/or a VAS rating obtained by the individual prior to receiving the implant and pain treatment. Other defecatory symptoms may be measured using other questionnaire-based scoring instruments including: Cleveland Clinic Constipation and Incontinence scores, St Marks Incontinence Score; the Patient Assessment of Constipation-Symptoms (PAC-SYM) questionnaire; fecal incontinence severity index (FISI).

1.-31. (canceled)

32. A system for treating pelvic pain of an individual, the system comprising:
one or more sensors implanted in a pelvic region of the individual and configured to sense a parameter associated with an episode of pelvic pain;
a stimulator electrode implanted in the pelvic region of the individual and configured to provide an adapted electrical stimulation; and
a processor operably coupled to the stimulator electrode and the one or more sensors;
wherein the processor is configured to:
receive from the one or more sensors the parameter associated with the episode of pelvic pain, the received parameter comprising a touch by the individual; and
in response to the received parameter, cause the stimulator electrode to provide the adapted electrical stimulation, wherein the adapted electrical stimulation provided is sufficient to reduce the pelvic pain of the individual.

33. The system of claim 32, wherein the one or more sensors comprises a sensor electrode.

34. The system of claim 32, wherein the one or more sensors comprises an accelerometer.

35. The system of claim 32, further comprising an implantable pulse generator configured to provide electrical simulation to the stimulator electrode, the implantable pulse generator comprising the processor.

36. The system of claim 35, wherein the processor is configured to cause the stimulator electrode to provide the adapted electrical stimulation in response to receiving the touch on the implantable pulse generator.

37. The system of claim 32, wherein the system is configured to provide a basal stimulation, and wherein the adapted electrical stimulation boosts the basal stimulation by providing one or more of a higher intensity, frequency or pulse width.

38. The system of claim 32, wherein the parameter is associated with at least one of contraction or increased tone of a pelvic floor muscle.

39. The system of claim 32, wherein the parameter is indicative of a change in posture.

40. The system of claim 32, wherein the adapted electrical stimulation is different when the individual wakes compared to when the individual sleeps.

41. The system of claim 32, wherein the adapted electrical stimulation is provided based on circadian functions indicative of when pelvic pain is worse.

42. The system of claim 32, wherein the adapted electrical stimulation is provided at or near a pudendal nerve.

43. The system of claim 32, wherein the adapted electrical stimulation is provided at or near a sacral nerve.

44. A system for treating pelvic pain of an individual, the system comprising:
a stimulator electrode implanted in a pelvic region of the individual and configured to provide an adapted electrical stimulation;
an interface configured to receive a touch from the individual based on perception of pain or increased pain; and
a processor operably coupled to the stimulator electrode and the interface;
wherein the processor is configured to:
receive an input from the interface indicative of the touch; and
in response to the received input, cause the stimulator electrode to provide the adapted electrical stimulation, wherein the adapted electrical stimulation provided is sufficient to reduce the pelvic pain of the individual.

45. The system of claim 44, wherein the interface comprises a user interface on a patient controller.

46. The system of claim 44, further comprising an implantable pulse generator configured to provide electrical simulation to the stimulator electrode, the implantable pulse generator comprising the processor.

47. The system of claim 46, wherein the interface comprises a surface of the implantable pulse generator.

48. The system of claim 44, wherein the system is configured to provide a basal stimulation, and wherein the adapted electrical stimulation boosts the basal stimulation by providing one or more of a higher intensity, frequency or pulse width.

49. The system of claim 44, wherein the adapted electrical stimulation is provided based on circadian functions indicative of when pelvic pain is worse.

50. The system of claim 44, wherein the adapted electrical stimulation is provided at or near a pudendal nerve.

51. The system of claim 44, wherein the adapted electrical stimulation is provided at or near a sacral nerve.

52. The system of claim 44, wherein the adapted electrical stimulation is different when the individual wakes compared to when the individual sleeps.
